(19) United States
(12) Patent Application Publication
(10) Pub. No.: US 2014/0019065 A1
(43) Pub. Date: Jan. 16, 2014

(54) INTERPOLATING A PORTION OF A SIGNAL IN RESPONSE TO A COMPONENT OF ANOTHER SIGNAL

(71) Applicant: TOKITAE LLC, Bellevue, WA (US)

(72) Inventor: Guillaume CHABOT-COUTURE, Seattle, WA (US)

(73) Assignee: TOKITAE LLC, Bellevue, WA (US)

(21) Appl. No.: 13/665,894

(22) Filed: Oct. 31, 2012

Related U.S. Application Data
(60) Provisional application No. 61/667,831, filed on Jul. 3, 2012.

(51) Int. Cl. G01C 15/00; G01N 33/00

(52) U.S. Cl. CPC G01C 15/00; G01N 33/0004
USPC 702/24; 702/150

(57) ABSTRACT

An embodiment of an apparatus includes a first component determiner configured to determine a component of a first signal, and an interpolator configured to interpolate a portion of the second signal in response to the component of the first signal. For example, such an apparatus may include an altitude-component determiner and an interpolator. The altitude-component determiner is configured to determine an altitude component of a first signal, and the interpolator is configured to interpolate an altitude component of a second signal in response to the altitude component of the first and to interpolate an empty portion of the second signal in response to the interpolated altitude component of the second signal.

If an Application Data Sheet (ADS) has been filed on the filing date of this application, it is incorporated by reference herein. Any applications claimed on the ADS for priority under 35 U.S.C. §§119, 120, 121, or 365(e), and any and all parent, grandparent, great-grandparent, etc. applications of such applications, are also incorporated by reference, including any priority claims made in those applications and any material incorporated by reference, to the extent such subject matter is not inconsistent herewith.

### CROSS-REFERENCE TO RELATED APPLICATIONS

The present application is related to and/or claims the benefit of the earliest available effective filing date(s) from the following listed application(s) (the “Priority Applications”), if any, listed below (e.g., claims earliest available priority dates for other than provisional patent applications or claims benefits under 35 USC §119(e) for provisional patent applications, for any and all parent, grandparent, great-grandparent, etc. applications of the Priority Application(s)). In addition, the present application is related to the “Related Applications,” if any, listed below.

### Priority Applications

For purposes of the USPTO extra-statutory requirements, the present application claims benefit of priority of U.S. Provisional Patent Application No. 60/667,831, entitled LAND-TEMP INTERPOLATION PROCEDURE ACCORDING TO AN EMBODIMENT, naming Guillaume Chabot Couture as inventor, filed Jul. 3, 2012, which was filed within the twelve months preceding the filing date of the present application or is an application of which a currently co-pending application is entitled to the benefit of the filing date.

### Related Applications

U.S. patent application Ser. No. TBD, entitled DETERMINING PORTIONS OF MULTIPLE SIGNALS ACCORDING TO RESPECTIVE ALGORTHMS, naming Guillaume Chabot Couture as inventor, filed TBD with attorney docket no. 2914-004-03, is related to the present application.
U.S. patent application Ser. No. TBD, entitled INTERPOLATING A PORTION OF A SIGNAL IN RESPONSE TO MULTIPLE COMPONENTS OF THE SIGNAL, naming Guillaume Chabot Couture as inventor, filed TBD with attorney docket no. 2914-005-03, is related to the present application.
U.S. patent application Ser. No. TBD, entitled INTERPOLATING A PORTION OF A SIGNAL IN RESPONSE TO A COMPONENT OF ANOTHER SIGNAL, naming Guillaume Chabot Couture as inventor, filed TBD with attorney docket no. 2914-006-03, is related to the present application.
U.S. patent application Ser. No. TBD, entitled INTERPOLATING A PORTION OF A SIGNAL IN RESPONSE TO A COMPONENT OF THE SIGNAL AND A COMPONENT OF ANOTHER SIGNAL, naming Guillaume Chabot Couture as inventor, filed TBD with attorney docket no. 2914-008-03, is related to the present application.

The United States Patent Office (USPTO) has published a notice to the effect that the USPTO's computer programs require that patent applicants reference both a serial number and indicate whether an application is a continuation, continuation-in-part, or divisional of a parent application. Stephen G. Kunin, Benefit of Prior-Filed Application, USPTO Official Gazette Mar. 18, 2003. The USPTO further has provided forms for the Application Data Sheet which allow automatic loading of bibliographic data but which require identification of each application as a continuation, continuation-in-part, or divisional of a parent application. The present Applicant Entity (hereinafter “Applicant”) has provided above a specific reference to the application(s) from which priority is being claimed as recited by statute. Applicant understands that the statute is unambiguous in its specific reference language and does not require either a serial number or any characterization, such as “continuation” or “continuation-in-part,” for claiming priority to U.S. patent applications. Notwithstanding the foregoing, Applicant understands that the USPTO's computer programs have certain data entry requirements, and hence Applicant has provided designation(s) of a relationship between the present application and its parent application(s) as set forth above and in any ADS filed in this application, but expressly points out that such designation(s) are not to be construed in any way as any type of commentary and/or admission as to whether or not the present application contains any new matter in addition to the matter of its parent application(s).

If the listings of applications provided above are inconsistent with the listings provided via an ADS, it is the intent of the Applicant to claim priority to each application that appears in the Priority Applications section of the ADS and to each application that appears in the Priority Applications section of this application.

All subject matter of the Priority Applications and the Related Applications and of any and all parent, grandparent, great-grandparent, etc. applications of the Priority Applications and the Related Applications, including any priority claims, is incorporated herein by reference to the extent such subject matter is not inconsistent herewith.

### SUMMARY

The foregoing summary is illustrative only and is not intended to be in any way limiting. In addition to the illustrative aspects, embodiments, and features described above, further aspects, embodiments, and features will become apparent by reference to the drawings and the following detailed description.

An embodiment of an apparatus includes a first component determiner configured to determine a component of a first signal, and an interpolator configured to interpolate a portion of a second signal in response to the component of the first signals.

An embodiment of such an apparatus includes an altitude-component determiner and an interpolator. The altitude-component determiner is configured to determine an altitude component of a first signal, and the interpolator is configured to interpolate an altitude component of the second signal in response to the altitude component of the first signal, and to interpolate an empty portion of the second signal in response to the interpolated altitude component of the second signal.

As compared to existing interpolation techniques such as interpolating an empty location of a signal directly from existing values of the signal or of another signal, a technique implemented by such an apparatus may, for example, interpolate an empty location of a signal more accurately by decomposing the signal and another signal into respective components, and by interpolating, at least partially, the empty location of the signal in response to components of both the signal and of the other signal. For example, when interpolating a dew-point value missing from a set of dew-point data, such an apparatus may decompose another set of dew-point data into its altitude component, determine a lapse-rate component for the set of dew-point data, and interpolate, at least partially, the missing dew-point value in response to the altitude component of the other set of dew-point data and the lapse-rate component of the set of dew-point data.

### BRIEF DESCRIPTION OF THE FIGURES

FIG. 1 is a diagram of a section of a land mass, and of a satellite that measures the temperatures of surface regions of the land-mass section, according to an embodiment.

FIG. 2 is a plot of “snapshots” of the land-mass section of FIG. 1 at respective surface-temperature-measurement times according to an embodiment.

FIG. 3 is a diagram of the land-mass section and of the satellite of FIG. 1, and of a phenomenon that prevents the satellite from measuring the temperature of a surface region of the land-mass section, according to an embodiment.

FIG. 4 is a plot of snapshots of the land-mass section of FIG. 3 at respective surface-temperature-measurement times, where the satellite of FIG. 3 did not generate valid temperature measurements for some of the surface regions at some measurement times, according to an embodiment.

FIG. 5 is a plot of a row of the snapshot grids of FIG. 4 at different surface-temperature-measurement times, with the measurement times and surface regions for which the satellite of FIGS. 1 and 3 did not generate valid surface-temperature measurements being identified, according to an embodiment.

FIG. 6 is a plot of a periodic seasonal component of the temperature measurements for one of the surface regions of FIGS. 1-5 according to an embodiment.

FIG. 7 is a plot of an aperiodic weather component of the temperature measurements for one of the surface regions of FIGS. 1-5 according to an embodiment.

FIG. 8 is a plot of a periodic noise component of the temperature measurements for one of the surface regions of FIGS. 1-5 according to an embodiment.

FIG. 9 is a plot of an aperiodic weather component of the temperature measurements for one of the surface regions of FIGS. 1-5, where the temperature measurements include identified empty portions e.g., missing or corrupted measurements), according to an embodiment.

FIG. 10 is a flow diagram of a technique for interpolating a portion of a signal, such as a temperature signal composed of temperature measurements, in response to components of the signal, according to an embodiment.

FIG. 11 is a flow diagram of a technique for interpolating a portion of a first signal, such as a first temperature signal, according to a first algorithm, and for interpolating a portion of a second signal, such as a second temperature signal, according to a second algorithm, according to an embodiment.

FIG. 12 is a flow diagram of a technique for interpolating a portion of a signal, such as a temperature signal composed of temperature measurements, in response to a component of another signal, according to an embodiment.

FIG. 13 is a flow diagram of a technique for interpolating a portion of a signal, such as a temperature signal composed of temperature measurements, in response to a component of the signal and a component of another signal, according to an embodiment.

FIG. 14 is a plot of snapshots of the land-mass section of FIG. 1 at respective surface-temperature-measurement times, with empty measurements identified, according to an embodiment.

FIG. 15 is a flow diagram of a technique for determining which one of multiple interpolation algorithms to use for interpolating empty portions of a signal, such as a temperature signal, according to an embodiment.

FIG. 16 is a diagram of a temperature signal for a surface region of FIGS. 1-5 and 14 according to an embodiment.

FIG. 17 is a flow diagram of a technique for interpolating empty portions of a non-pathological signal (e.g., a signal with a number of non-empty portions sufficient to determine a component or outliers of the signal), such as a non-pathological temperature signal, according to an embodiment.

FIG. 18 is plot of an array of temperature data, and of a “window” for determining a respective standard deviation of the data at each temperature-data-measurement time, according to an embodiment.

FIG. 19 is a flow diagram of a technique for interpolating empty portions of a pathological signal (e.g., a signal with a number of non-empty portions insufficient to determine a component or outliers of the signal), such as a pathological temperature signal, according to an embodiment.

FIG. 20 is a plot of snapshots of surface regions of a land-mass section at respective air-temperature measurement times, where at least one of the surface regions includes a respective weather station, according to an embodiment.

FIG. 21 is a plot of a semi-variogram model, and of values, such as air-temperature residuals, to which the model is fitted, according to an embodiment.

FIG. 22 is a flow diagram of a technique for interpolating a value, such as an air-temperature value, for each empty portion of a signal, such as an air-temperature signal, according to an embodiment.

FIG. 23 is a plot of snapshots of surface regions of a land-mass section at respective dew-point measurement times, where at least one of the surface regions includes a respective weather station, according to an embodiment.

FIG. 24 is a plot of dew points versus altitudes of the respective weather stations that measured the dew points, a curve fitted to the points, and a filtered version of the fitted curve, according to an embodiment.

FIG. 25 is a plot of a semi-variogram model, and of values, such as altitude-compensated dew-point measurements, to which the model is fitted, according to an embodiment.

FIG. 26 is a flow diagram of a technique for interpolating values, such as dew-point values, for the empty portions of a signal, such as a dew-point signal, according to an embodiment.

FIG. 27 is a flow-diagram of a technique for determining values, such as relative-humidity values, according to an embodiment.

FIG. 28 is a functional block diagram of a computing apparatus that is configured to implement one or more steps of an interpolation technique according to an embodiment.

FIG. 29 is a block diagram of an embodiment of an apparatus/tool for simulating a stochastic system in response to data interpolated, or otherwise generated, according to one or more of the techniques described in conjunction with FIGS. 10-13, 15, 17, 19, 22, and 26-27.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

One or more embodiments are described with reference to the drawings, wherein like reference numerals may be used to refer to like elements throughout. In the following description, for purposes of explanation, numerous specific details are set forth in order to provide a thorough understanding of the one or more embodiments. It may be evident, however, that one or more embodiments may be practiced without these specific details. In other instances, well-known structures and devices are shown in block-diagram form in order to facilitate describing one or more embodiments.

A set of data values or elements may be in the form of a discrete signal, a vector, or a multidimensional array, one or more of the elements may be omitted or may be otherwise invalid, and one or more of the present/valid elements may include an error. The omitted/invalid elements may be referred to as “empty” elements, or as “empty” portions, of the set of data elements, and the present/valid elements may be referred to as “non-empty” elements, or as “non-empty” portions, of the set of data elements. Causes of empty data-set portions, and of errors in non-empty data-set portions, may include a temporary failure of equipment used to generate the data set, the temporary inability of the equipment to acquire a data element due to, e.g., an environmental disturbance, or noise.

Because one may use such data sets to predict, simulate, or otherwise analyze related physical phenomena such as weather patterns, the movement of celestial objects, or the simulation of the spreading of disease or of campaigns to eradicate disease, scientists, mathematicians, and engineers have developed techniques for estimating the empty portions of a data set and for correcting errors in the non-empty portions of the data set.

Interpolation is a general category of such estimation techniques by which a person or a computing apparatus may estimate a value for an empty portion of a data set from non-empty portions (i.e., from present/valid elements or from previously interpolated elements) of the data set.

In general, an interpolation technique includes analyzing the valid elements of a data set, and using the results of this analysis to generate relatively accurate estimates of the empty elements of the data set, where the required level of the estimates' accuracy depends on the application that will use the interpolated data set. For example, consider the data vector [7, X, −3], where “X” indicates an empty element. If it is known or determined that the elements of this data vector lie approximately along a straight line and are approximately evenly spaced from one another, then one may interpolate the value of the empty element as being halfway between the two valid elements according to the following equation:

X=7−(7−(−3))/2=−3+(7−(−3))/2=2 (1)

That is, the empty element X is interpolated as being a value, 2, which is five units away from both 7 and −3.

Similarly, an error-correcting technique includes analyzing the valid elements of a data set, and using the results of this analysis to identify and correct erroneous, or otherwise invalid, non-empty elements of the data set, where the required level of the corrected elements' accuracy depends on the application that will use the corrected data set. For example, consider the data vector [7, 18, −3]. If it is known or determined that the elements of this data vector lie approximately along a straight line and are approximately evenly spaced from one another, then one may identify the middle element, 18, as being erroneous, or as being an outlier, and may determine that the correct value of this erroneous middle element is a value that is halfway between the two valid end elements, 7 and −3, according to equation (1). That is, the erroneous middle element is corrected to have a value, 2, which is five units away from both of the valid end elements, 7 and −3. Although in this example the correction of the middle element uses the interpolation technique described above, error-correction techniques that do not involve interpolation are also available.

An example of a real-world application that may use interpolated and corrected data sets is the simulation of a disease-eradication campaign,

U.S. patent application Ser. No. 13/199,040, entitled Determining A Next Value Of a Parameter For System Simulation, filed Aug. 16, 2011, Ser. No. 13/199,044, entitled Determining a Next Value Of a System-Simulation Parameter In Response To a Representation Of a Plot Having the Parameter as a Dimension, filed Aug. 16, 2011, and Ser. No. 13/199,039, entitled Determining a Next Value Of a System-Simulation Parameter In Response To Representations Of Plots Having the Parameter as a Dimension, filed Aug. 16, 2011, which are incorporated by reference, disclose a simulator for simulating, and for predicting the success rate of, a disease-eradication campaign, where the results of the campaign depend, at least in part, on weather phenomena, such as, but not limited to, land temperature, air temperature, dew point, relative humidity, and rain fall in a region for which the simulator simulates the campaign.

To simulate a campaign to eradicate a disease from a region, a simulator can model the disease and campaign as a system that receives, as inputs, sets of data that correspond to the region, including sets of data that are related to weather phenomena. For example, to simulate a campaign to eradicate malaria from a region, the simulator may model the incubation, transmission, and remediation processes for malaria as a complex stochastic system that receives, as inputs, sets of weather-related data (e.g., measured land and air temperatures, dew points, relative humidities, and levels of rain fall) for the region.

Because one or more empty portions of a data set, such as a weather-related data set, can reduce the accuracy of a disease-eradication simulation, or even prevent the simulator from performing such a simulation, the simulator, or another computing apparatus, can use one or more techniques for interpolating values for these empty portions.

But because a disease-eradication system model can include states that have a relatively high sensitivity to the values of data on which the state depends, even relatively small errors in the interpolated values of a data set may significantly reduce the accuracy with which a simulator can simulate the results of a disease-eradication campaign.

Therefore, described below are embodiments of interpolation techniques that may yield interpolated weather-data values that are accurate enough for use by a disease-eradication-campaign simulator, such as the simulator described in U.S. patent application. Ser. Nos. 13/199,040, 13/199,044, and 13/199,039, which were previously incorporated by reference.

Furthermore, even valid weather-data values may include errors due to, e.g., noise, and these errors may also reduce the accuracy with which a simulator can simulate the results of a disease-eradication campaign.

Therefore, also described below are embodiments of error-correction techniques for yielding corrected weather-data values that are accurate enough for use by a disease-eradication-campaign simulator, such as the simulator described in U.S. patent application Ser. Nos. 13/199,040, 13/199,044, and 13/199,039, which were previously incorporated by reference.

Although these and other embodiments are respectively described relative to the interpolation or correction of weather-data values, these descriptions are liar example purposes; therefore, it is contemplated that the below-described embodiments of interpolation and error-correction techniques may be useful for interpolating and correcting values other than weather-data values.

But before describing embodiments for respectively interpolating and correcting weather-data values, described are examples of weather-data sets that may include empty portions and errors, examples of the generation of such weather-data sets, and examples of sources of empty portions and errors in such weather-data sets.

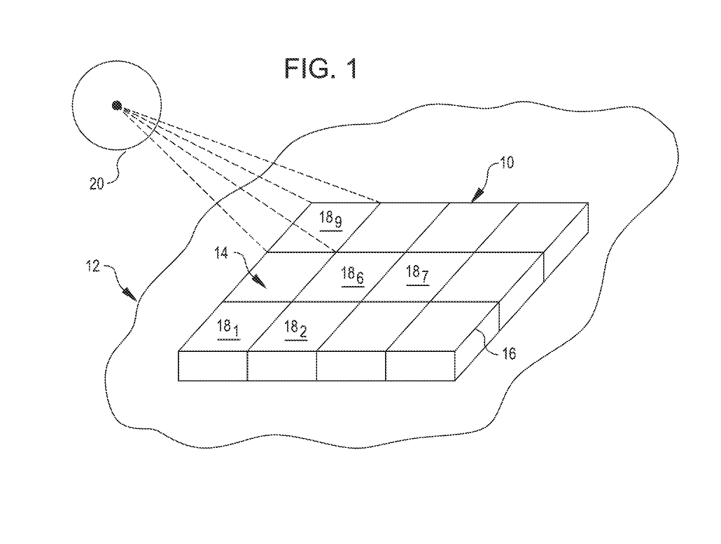

FIG. 1 is a diagram of a section 10 of a landmass 12 having a surface 14 that is divided, according to a grid pattern 16, into surface regions 18 (twelve surface regions in this example), and is a diagram of a satellite 20 for measuring the temperatures of the surface regions, according to an embodiment. Although the surface 14 is shown as being smooth, it may include “rough” geological features such as mountains, valleys, or canyons, and may include bodies of water such as lakes and rivers. Furthermore, the surface regions 18 may each have a uniform area that corresponds to the temperature-measurement resolution of the satellite 20, although surface regions having non-uniform areas are contemplated. Moreover, because one may liken the surface regions 18 of the landmass section 10 to the picture elements of a digital image, the surface regions may also be called “pixels.” in addition, the satellite 20 may be the National Aeronautic and Space Administration's (NASA's) AQUA satellite, which is in a sun-synchronous orbit around the earth, and which periodically measures the temperature of each of the surface regions 18. For example, as further discussed below, the AQUA satellite measures the temperature of each surface region 18 twice per day at approximately twelve-hour intervals, and the temperature-measurement resolution of the AQUA satellite, and, therefore, the area of each surface region when the AQUA satellite is relied upon to measure the surface temperature of each region is approximately 1.0 km².

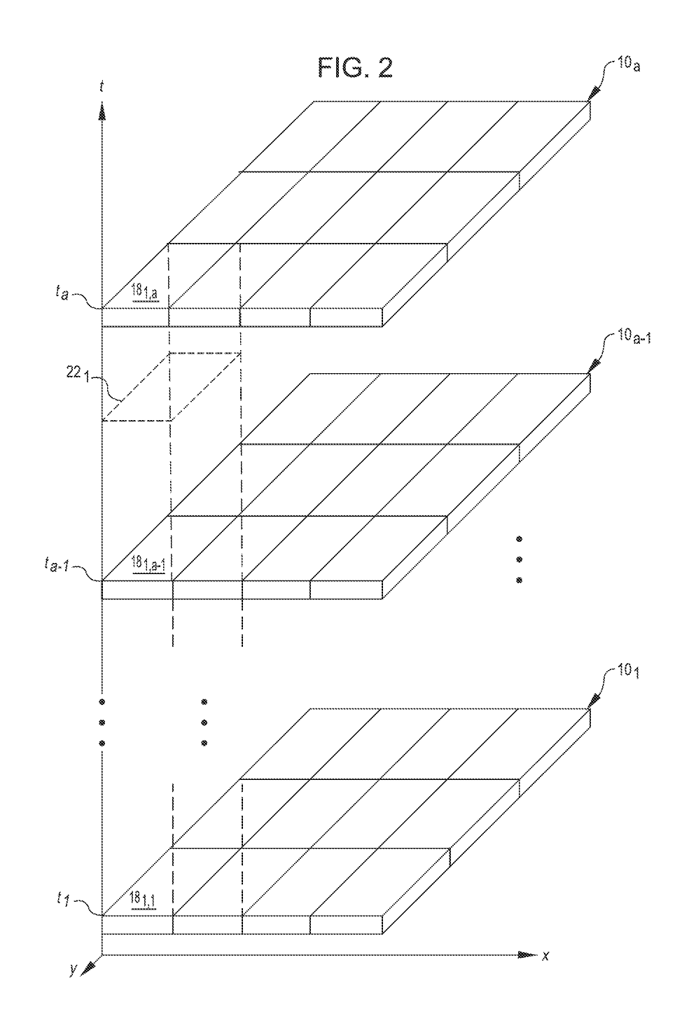

FIG. 2 is a plot of the landmass section 10 of FIG. 1 versus time, with each “snapshot” 10₁-10ₐ of the landmass section representing a respective temperature-measurement, i.e., “sample,” time t₁-t_{α} when the satellite 20 (FIG. 1) measures the temperatures of the surface regions 18. For purposes of explanation, it is assumed that at each sample time t, the satellite measures the temperatures of the surface regions 18 of the landmass section 10 simultaneously, although there may be a small difference between the time at which the satellite measures the temperature of a surface region and the time at which the satellite measures the temperature of an adjacent surface region; therefore, hereinafter, unless otherwise stated, it is assumed that these time differences are small enough to be considered negligible, at least for the described applications.

Referring to FIGS. 1 and 2, the set of satellite 20 temperature measurements for each surface region 18, which set can he represented by a respective column 22 (FIG. 2) of surface-region snapshots, forms a data set, i.e., a signal, or vector, of data elements, where each data element is a temperature of the surface region measured at a respective sample time t. For example, the column 22₁ of the snapshots 18_{1,1}-18_{1,a} of the surface region 18₁ corresponds to an α×1 column vector [Temp_{1,1}, Temp_{1,2}, . . . , Temp_{1, α-1}, Temp_{1,α}]^{T} of temperatures of the surface region 18₁ that the satellite 20 measured at the respective sample times t₁-t_{α}.

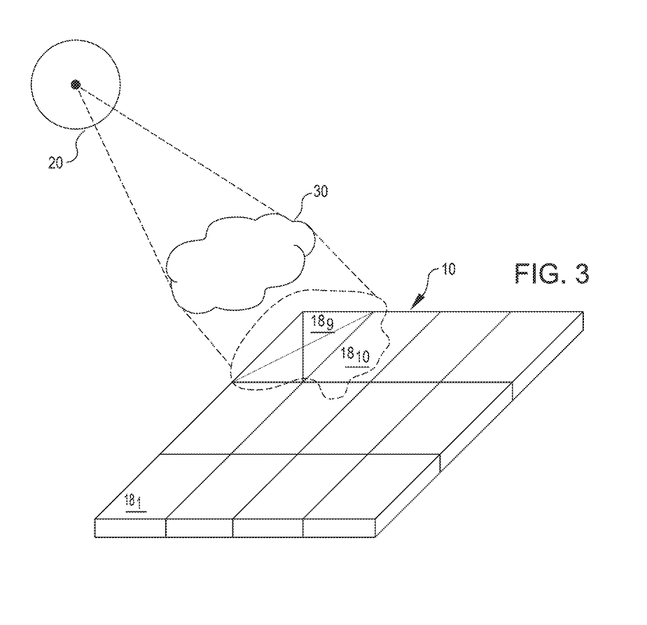

FIG. 3 is a diagram of the landmass section 10 and the satellite 20 of FIG. 1, and of an environmental phenomenon 30, which may render the satellite temporarily unable to measure the temperature of one or more of the surface regions 18, according to an embodiment. For example, if the satellite 20 measures the temperature of a surface region 18 by analyzing electromagnetic radiation emanating from the surface region, then an environmental phenomenon 30 that prevents such radiation from reaching the satellite, or that otherwise significantly distorts such radiation, may render the satellite unable to measure the temperature of he surface region while the phenomenon persists.

For example, where the environmental phenomenon 30 is cloud cover, then the satellite 20 may be unable to measure the temperature of the surface region 18₉ while the cloud cover persists between the satellite and the surface region; therefore, the region 18₉ is marked with an “X” to indicate that the one or more temperature-measurement data elements corresponding to the region 18₉ at sample times t during which the cloud cover persists are “empty,” i.e., form “holes” in the surface-temperature data set corresponding to the region 18₉. If the cloud cover 30 covers only part of a surface region, then whether the cloud cover renders the satellite 20 unable to measure the temperature of the region may depend on the percentage of the region that the clouds covers. For example, even though the clouds 30 cover a portion of the surface region 18₁₀, the satellite 20 is still able to measure the temperature of this surface region as long as the clouds do not cover more than a threshold percentage (e.g., 50%) of this surface region. Examples of the environmental phenomenon 30 other than cloud cover include weather disturbances such as storms, solar flares, and other solar-radiation events.

Still referring to FIG. 3, phenomena, other than environmental phenomena, that may render the satellite 20 temporarily unable to measure the temperature of a surface region 18, and, therefore, that may spawn an empty temperature measurement, include a malfunction of the satellite, a test or upgrade of the satellite software or firmware, a power glitch, and a single-event upset (SEU) caused by radiation striking the satellite circuitry.

Furthermore, although the satellite 20 can sometimes generate a temperature measurement for a surface region 18 even when the phenomenon 30 is present, the satellite circuitry may conventionally flag this generated temperature measurement as being invalid based on the level of interference caused by the phenomenon; therefore, at least for purposes of interpolation, a computing apparatus can consider an invalid temperature measurement to be an empty temperature measurement.

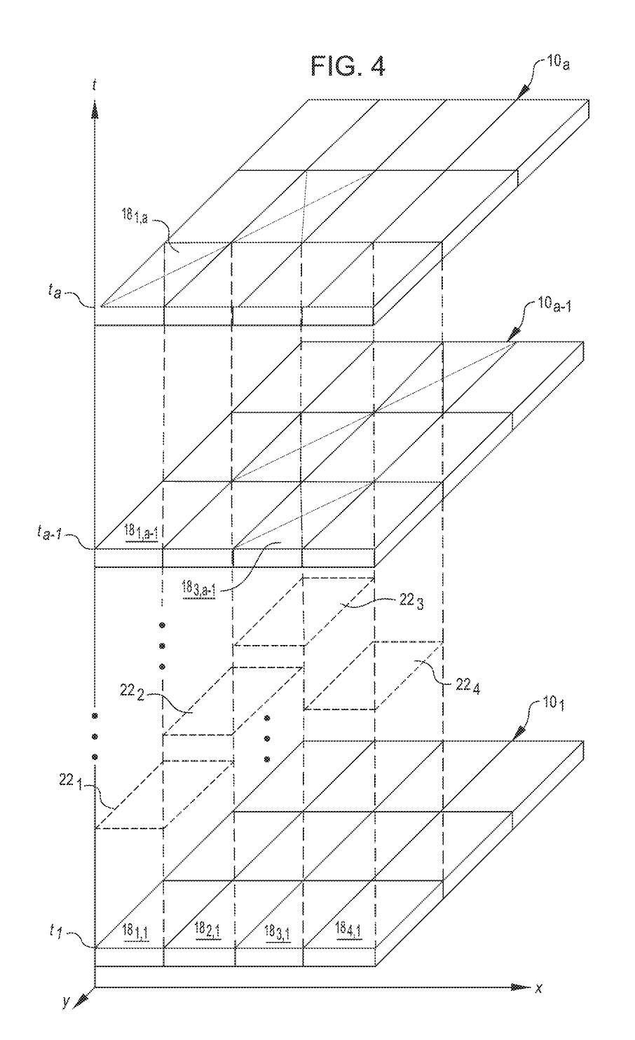

FIG. 4 is a plot of the landmass section 10 of FIG. 3 versus time, with each snapshot 10₁-10ₐ of the landmass section representing a respective sample time t₁-t_{α} when the satellite 20 (FIG. 1) measures the temperatures of the surface regions 18, and where “X” is used to indicate surface regions that yield empty temperature measurements at corresponding snapshot-sample-times t—to yield an empty temperature measurement means that the satellite 20 (FIG. 3) was unable to measure a valid temperature for an X-marked surface region at the corresponding sample time t. For example, the surface region 18₁ yields an empty temperature measurement at sample time t_{α}; therefore, the corresponding snapshot 18_{1,a} of this surface region is labeled with an. “X”. Likewise, the surface region 18₃ yields an empty temperature measurement at sample time t_{α-1}; therefore, the corresponding snapshot 18_{3,a-1} of this surface region is also labeled with an “X”. And because no surface region 18 yields an empty temperature measurement at sample time t₁, none of the surface-region snapshots within the landmass-section snapshot 10₁ are labeled with an “X”.

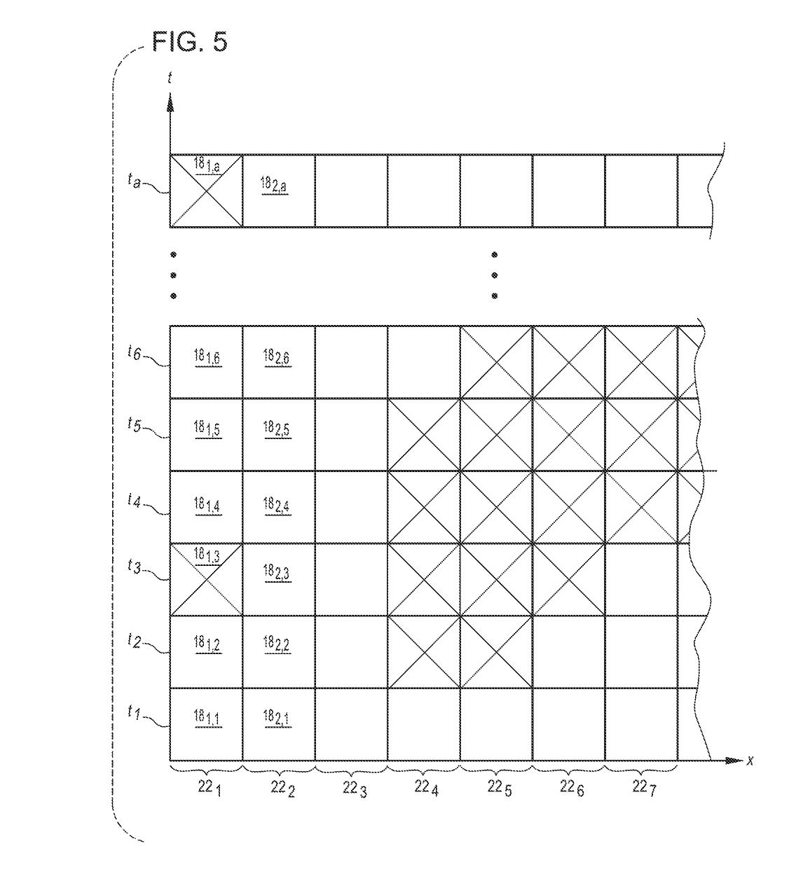

FIG. 5 is a plot of columns 22₁-22₇ in the first row of the landmass snapshots 10₁-10ₐ of FIG. 4 according to an embodiment. FIG. 5 includes more columns 22 in the first row than does FIG. 4 (seven columns 22₁-22₇ in FIG. 5 versus only four columns 22₁-22₄ in FIG. 4), and thus includes more surface regions 18 than does FIG. 4, for example purposes; but it is understood that the landmass section 10 may include any number of surface regions 18 that is suitable for a particular application.

The temperature measurements associated with each column 22 form an α×1 column vector of temperature values for the surface region 18 that is associated with the column, and, as described above in conjunction with FIG. 4, “X” indicates an empty element of this column vector. For example, the temperature measurements associated with the column 22₁ form an α×1 column vector of temperature values for the surface region 18₁, the temperature measurements associated with the column 22₂ form an α×1 column vector of temperature values for the surface region 18₂, and so on.

One may also think of a column vector of temperature values as forming a digital signal having samples (the temperature values) at the respective sample times t₁-t_{α}.

Furthermore, the number a of rows, and thus values, in such an α×1 vector can depend on the application. For example, as discussed above, if the satellite 20 is the NASA AQUA satellite, then, ideally, it measures the temperature of each surface region 18 twice per day at approximately 12-hour intervals, typically one nighttime temperature reading and one daytime temperature reading. So if a column 22 represents ten years of AQUA temperature measurements, then α=2·10·365+LY≈7300, where LY equals the number of leap years within the ten-year period of temperature measurements, and thus equals either two or three in this example. Therefore, the column vector of temperature values for the corresponding surface region 18 includes α≈7300 elements, some of which may be empty. Alternatively, if an application uses only the AQUA daytime temperature readings, then the column vector of temperature values for the corresponding surface region 18 includes α≈3650 elements, some of which may be empty.

As described below, an embodiment of an interpolation technique entails partially interpolating empty elements of a temperature vector that corresponds to a first column 22 in response to valid temperature values within the same temperature vector (temporal interpolation), and entails partially interpolating the empty elements in response to valid temperature values within one or more vectors that respectively correspond to one or more second columns 22 that are adjacent to, or that are otherwise near to, the first column 22 (spatial interpolation).

Still before describing embodiments of interpolation and error-correction techniques, the concepts of noise and signal components are discussed as they relate to data sets such as those described above. Understanding these concepts should facilitate the understanding of the embodiments of the interpolation techniques and error-correction techniques to be described below.

Referring again to FIGS. 1-5, non-empty data elements within a set of data elements, such as non-empty temperature measurements in a vector of temperature measurements generated by the satellite 20, may also be corrupted by noise. For example, as discussed above, NASA's AQUA satellite is in a sun-synchronous orbit that causes the AQUA satellite to be approximately over a same region of the earth's surface twice each day at approximately twelve-hour intervals. For example, the AQUA satellite may cross the equator twice each day at twelve-hour intervals, where each crossing is over approximately the same location, but is not over exactly the same location, as was the immediately prior crossing. Consequently, because each equator crossing of the AQUA satellite is at a slightly different location than the immediately prior crossing, the AQUA satellite measures the temperature of each surface region of the earth from a slightly different angle relative to the previous temperature measurement. And because a different measurement angle may affect the measured temperature value for a surface region, each measured temperature value for a surface region may include a measurement-angle-induced error component caused by the AQUA satellite not crossing over the equator (or other given latitude) at exactly the same location every twelve hours. As further described below, one may model this error component as noise that is superimposed on the uncorrupted temperatures that the AQUA satellite would have measured but for this orbit-induced noise.

Unfortunately, if a computing apparatus interpolates empty elements of a data set with noise-corrupted elements of the data set or of another data set, then not only are the non-empty elements corrupted by noise, but the interpolated elements may also be corrupted by noise.

Consequently, as further described below, an embodiment of an interpolation technique includes a technique for filtering noise, such as periodic noise, from non-empty data elements in a data set before using these data elements to interpolate empty elements in the data set or in another data set. Therefore, this filtering technique not only filters noise from the non-empty data elements, it also prevents this filtered noise from corrupting the interpolated data elements.

As alluded to above, NASA's AQUA satellite is an example of an apparatus that introduces periodic noise into the data sets (here column vectors of surface-region temperature measurements as described above) that it produces. The AQUA satellite orbits the earth 14.5625 times per day. To cross a same surface region of the earth every twelve hours, it can be shown that a satellite, such as the AQUA satellite, must orbit the earth 2n+1 times per day, where n is any nonnegative integer. That is, if the AQUA satellite were to circle the earth 2·7+1=15 times per day, then it would not introduce the above-described orbit-induced noise into its surface-region temperature measurements. But the number of times that the AQUA satellite circles the earth per day falls short of fifteen by 0.4375. So starting at an initial time zero when the AQUA satellite is over a surface region of the earth, it is known that this surface region will be in the same location, relative to the earth's rotation, once every twenty four hours, i.e., once per day (for example purposes, it is assumed that the earth rotates once about its axis exactly every twenty four hours). It is also known that the AQUA satellite will be in the same location, relative to its orbit, every complete revolution around the earth. Therefore, to determine the next time when the locations of the satellite and the surface region will again coincide, one solves the equation m·14.5625=n for the smallest value of in where both m and n are integers. For the AQUA satellite, the solution to this equation is m=16 and n=233, which means that every sixteen days the AQUA satellite circles the earth 233 times and crosses over the same surface region of the earth at the same local time. So, the even though the AQUA satellite circles the earth once every 98.4 minutes, it also has a repeatable period of sixteen days. And although NASA and others know that this 16-day period introduces noise into AQUA's temperature measurements, NASA purposely launched the AQUA satellite into its noise-introducing orbit so that the satellite can measure temperatures over the entire portion of the earth's surface along the equator; the details of how this orbit allows the satellite to measure surface temperatures along the entire equator are omitted for brevity.

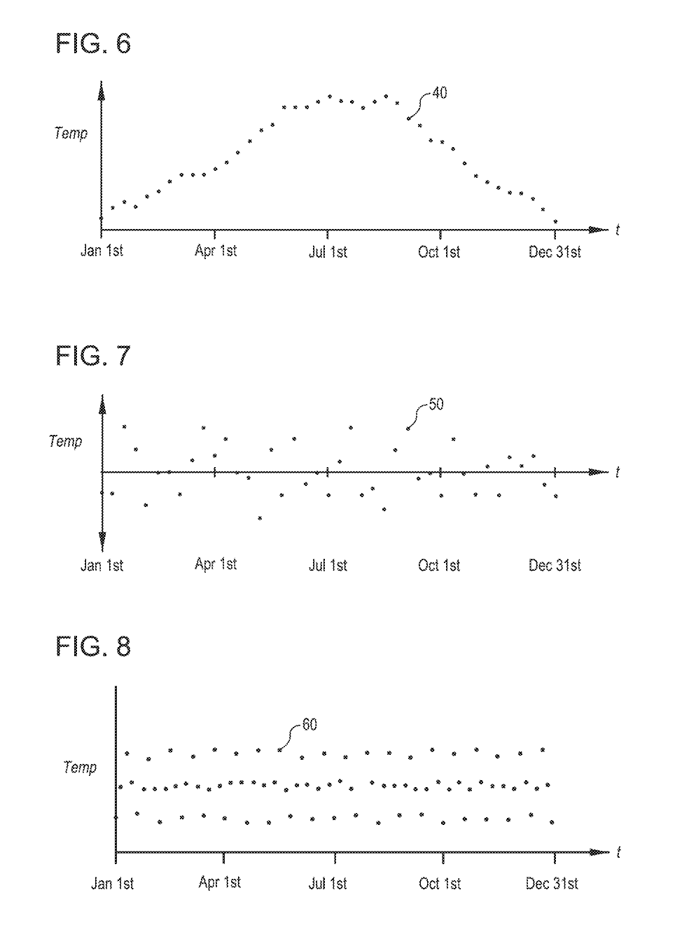

Furthermore, referring to FIGS. 6-8, which are described below, because a set of data elements may form a digital data-element signal as described above, such a signal may be decomposed into one or more signal components. Again, for example purposes, the decomposition of a data-element signal into one or more signal components is described with reference to a digital temperature signal formed by a vector of temperature measurements that the satellite 20 (FIGS. 1 and 3) takes of a surface region 18 (FIGS. 1-5) of the landmass section 10 (FIGS. 1-4).

It is known that for many locations in the earth's northern hemisphere, the actual land temperatures follow a seasonal pattern that generally repeats itself every year. That is, during the winter the temperatures are generally at their lowest levels, during the summer the temperatures are generally at their highest levels, and during the spring and autumn the temperatures are generally midway between their highest and lowest levels. Therefore, as winter gives way to spring, and spring gives way to summer, the land temperatures gradually increase from their lowest levels to their highest levels, and as summer gives way to autumn, and autumn gives way to winter, the land temperatures gradually decrease from their highest levels to their lowest levels.

In addition to this general seasonal pattern, the land temperatures may experience random, weather-related fluctuations that are much less gradual than the seasonal variations described above. For example, there may be a few days in January that are significantly warmer than normal due to the presence of a slow-moving high-pressure system that establishes airflow from the south, or that are significantly colder than normal due to a cold front moving through; similarly, there may be days in July that are significantly cooler than normal due to a storm system, or that are significantly warmer than normal due to a heat wave. Moreover, there may be few spring days that are colder than normal due to a spring snowstorm, or a few autumn days that are wanner than normal due to an “Indian Summer.”

Consequently, the temperature signal formed by the actual temperatures of, e.g., a surface region 18 (FIGS. 1-5) of the landmass section 10, may be decomposed into at least two components: a seasonal component having a fundamental period of one year, and a weather component that is not periodic, i.e., that is aperiodic.

Furthermore, as discussed above, the apparatus (e.g., the satellite 20 of FIGS. 1 and 3) that measures the surface-region temperatures may introduce noise into these measured temperatures. Therefore, in addition to the periodic seasonal and the aperiodic weather components, the temperature signal formed by the measured temperatures of e.g., a surface region 18 (FIGS. 1-5), may include a noise component that is periodic or aperiodic.

FIG. 6 is a plot of a periodic seasonal component 40 of a temperature signal generated by the satellite 20 (e.g., NASA's AQUA satellite) of FIGS. 1 and 3 over the course of one year for a surface region 18 (FIGS. 1-5) of the landmass section 10 (FIGS. 1 and 3) according to an embodiment, where the landmass section is located in the earth's northern hemisphere, and it is assumed that the temperature signal has no empty portions. As one may expect per the above discussion of the seasonal temperature component being periodic, the periodic seasonal component 40 has a generally sinusoidal shape.

FIG. 7 is a plot of an aperiodic weather component 50 of the same satellite-generated temperature signal for which the periodic seasonal component 40 is plotted in FIG. 6, according to an embodiment. As one may expect per the above discussion of the weather component of the temperature signal being aperiodic, the aperiodic weather component 50 has a generally random stochastic distribution.

FIG. 8 is a plot of a periodic noise component 60 of the same satellite-generated temperature signal for which the periodic seasonal and aperiodic weather components 40 and 50 are respectively plotted in FIGS. 6 and 7, according to an embodiment. For example, where the satellite 20 is NASA's AQUA satellite, the predominant fundamental period of the noise component 60 is sixteen days. Furthermore, it has been discovered that the AQUA satellite also introduces into its land-temperature measurements significant noise at periods of 1781/4096 days and 1803/4906 days; therefore, the noise component 60 has energy at these fundamental periods as well. Moreover, although the AQUA satellite may introduce aperiodic noise, such as Additive White Gaussian Noise (AWGN), into its land-temperature measurements, such aperiodic noise is not plotted as part of the noise component 60, and is hereinafter assumed to be negligible for the described applications unless otherwise stated.

Referring to FIGS. 6-8, a theory behind an embodiment of an interpolation technique is that if a signal, such as a temperature signal, has one or more periodic components and aperiodic components, then the periodic components are the same for both empty and non-empty portions of the signal, and only the aperiodic components differ from signal sample to signal sample.

Consequently, according to this theory, if a computing apparatus can determine the one or more periodic components of the signal from the non-empty portions, i.e., the non-empty samples or non-empty elements, and determine the aperiodic components corresponding to the non-empty portions, then the computing apparatus need only interpolate the aperiodic components corresponding to the empty portions and add these interpolated aperiodic components to the periodic components to fully interpolate the empty portions of the signal.

Because, according to this theory, a computing apparatus may need only to interpolate a component of the empty portions of a signal, an interpolation technique based on this theory may yield more accurate results than a conventional technique that calls for interpolating empty portions from the values of only non-empty portions using, e.g., simple linear interpolation.

A general concept of interpolating empty portions of a signal by interpolating one or more components of the signal is further described below in conjunction with FIGS. 6 and 9-10.

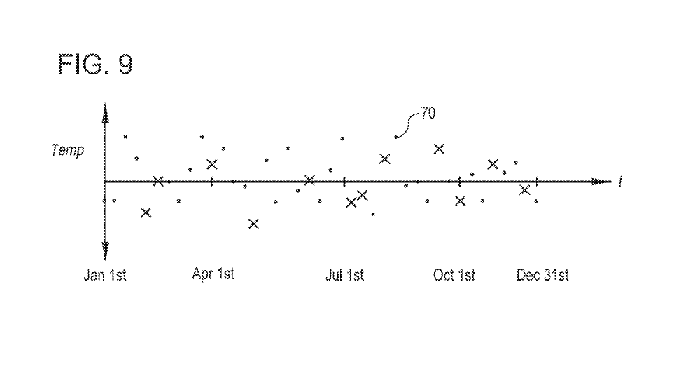

FIG. 9 is a plot of an aperiodic weather component 70 of a satellite-generated temperature signal according to an embodiment, where “X” indicates empty portions of the aperiodic weather component that correspond to the empty portions (i.e., empty temperature values) of the temperature signal.

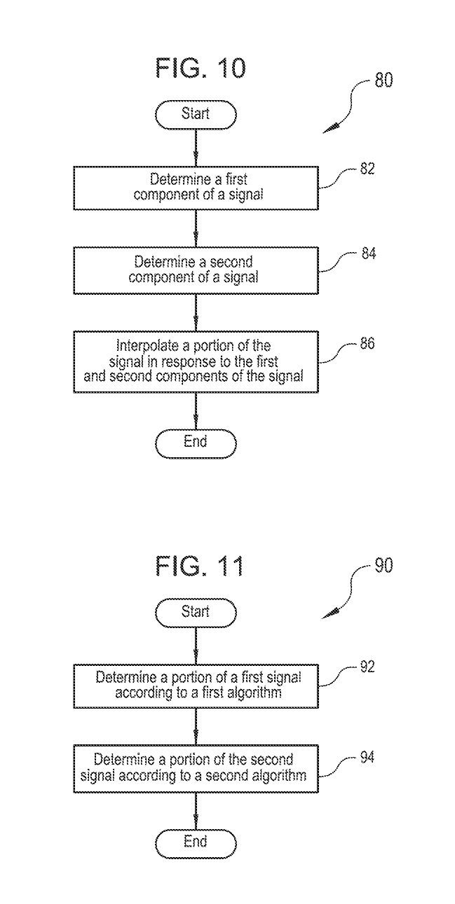

FIG. 10 is a flow diagram 80 of an embodiment of a general algorithm for interpolating one or more portions (e.g., empty portions) of a signal by interpolating a corresponding one or more portions (e.g., empty portions) of a component of the signal. For example, the signal may be a satellite-generated temperature signal having at least one periodic component, such as the periodic seasonal component 60 of FIG. 6, and having at least one aperiodic component, such as the aperiodic component 70 of FIG. 9.

Referring to step 82, a computing apparatus (not shown in FIG. 10) first determines a first component of the signal having at least one empty portion, where the first component has no empty portions. For example, referring to FIG. 6, the computing apparatus may first determine as the first component a periodic component, such as the periodic seasonal component 40, of a temperature signal from the non-empty portions of the signal.

Next, referring to step 84, the computing apparatus determines a second component of the signal, where the second component has a respective empty portion that corresponds to each empty portion of the signal. For example, referring to FIG. 9, the computing apparatus may determine, as the second component, an aperiodic weather component, such as the component 70, of a temperature signal by subtracting from the non-empty portions of the temperature signal the respective portions of a periodic seasonal component, such as the component 40 of FIG. 6, of the signal. In addition, the computing apparatus may also correct the second component. For example the computing apparatus may filter noise from the second component by subtracting from the non-empty portions of the second component the corresponding portions of a noise component, such as the periodic noise component 60 of FIG. 8, of the temperature signal.

Then, referring to step 86, the computing apparatus interpolates the one or more empty portions of the signal in response to the first and second components of the signal. For example, referring to FIGS. 6 and 9, the computing apparatus may first interpolate the empty samples of the aperiodic weather component 70 (the second component) in response to the non-empty samples of the aperiodic weather component, and then add the samples (both the interpolated and the original samples) of the aperiodic weather component to the corresponding samples of the periodic seasonal component 40 (first component) to obtain a resulting temperature signal having interpolated samples in the locations of the formerly empty samples of the signal. And if the computing apparatus corrects the second component of the temperature signal by, e.g., filtering noise from the second component as discussed above in conjunction with step 84, then all samples of the resulting interpolated temperature signal are also corrected.

Alternate embodiments of interpolation algorithm of FIG. 10 are contemplated. For example, the algorithm may include fewer or more steps than described, and these steps may be performed in any suitable order.

Still referring to FIG. 10, the computing apparatus may be able to perform the described interpolation procedure only if the non-empty portions of the signal provide information sufficient to determine at least one component of the signal. For example, if a temperature signal formed by daily temperature measurements of a surface region 18 (FIGS. 1-5) over the course of one year includes only one non-empty portion, then this single non-empty portion may provide a level of information that is insufficient to determine a periodic seasonal component of the temperature signal.

Consequently, if the non-empty portions of a signal provide information that is insufficient for the embodiment of the interpolation technique described in conjunction with FIG. 10, then the computing apparatus may need to implement another technique for interpolating the empty portions of the signal.

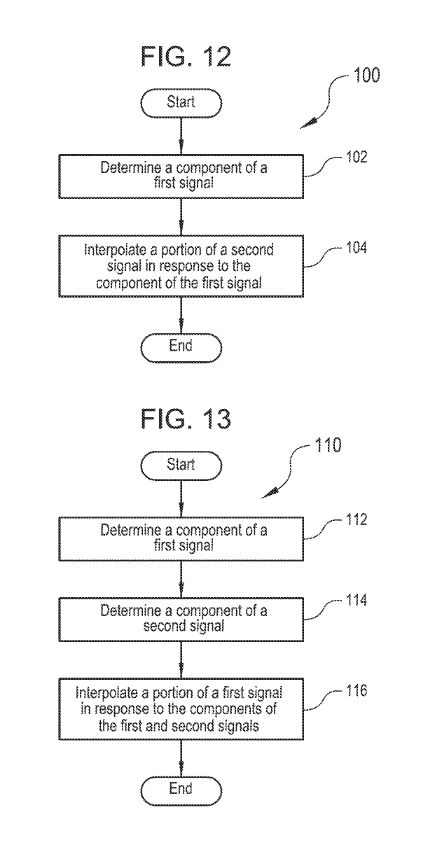

FIG. 11 is a flow diagram 90 of an embodiment of an algorithm for determining a portion of a first signal according to a first algorithm, and determining a portion of a second signal according to a second algorithm. For example, a computing apparatus (not shown in FIG. 11) may analyze first and second temperature signals, and may determine that the non-empty portions of the first temperature signal include information sufficient to determine a periodic, seasonal component of the first temperature signal, but that the non-empty portions of the second temperature signal do not include information sufficient to determine a periodic seasonal component of the second temperature signal. Consequently, the computing apparatus may interpolate the empty portions of the first signal according to the interpolation algorithm described above in conjunction with FIG. 10, and may interpolate the empty portions of the second signal according to another interpolation algorithm such as the algorithm described below in conjunction with FIG. 12.

Still referring to FIG. 11, at step 92, the computing apparatus determines (e.g., interpolates) a portion (e.g., an empty portion) of a first signal according to a first algorithm (e.g., the interpolation algorithm described above in conjunction with FIG. 10).

And at step 94, the computing apparatus determines (e.g., interpolates) a portion (e.g., an empty portion) of a second signal according to a second algorithm (e.g., the interpolation algorithm described below in conjunction with FIG. 12).

Alternate embodiments of the algorithm of FIG. 11 are contemplated. For example, the algorithm may include fewer or more steps than described, and these steps may be performed in any suitable order.

Furthermore, a general concept of interpolating empty portions of a first signal in response to one or more components of a second signal is described below in conjunction with FIGS. 9 and 12.

FIG. 12 is a flow diagram 100 of an embodiment of a general algorithm for interpolating one or more portions (e.g., empty portions) of a second signal, such as a second satellite-generated temperature signal, from one or more components, such as the periodic seasonal and aperiodic weather components 40 (FIG. 6) and 70 (FIG. 9), of a first signal, such as a first satellite-generated temperature signal; for example, the first temperature signal may correspond to a first surface region 18 (FIGS. 1-5), and the second temperature signal may correspond to a second surface region 18 that is adjacent to, or that is otherwise near, the first surface region. The algorithm may be suitable for interpolating empty portions of a signal whose non-empty portions lack information sufficient to determine one or more components of the signal as discussed above in conjunction with FIGS. 10-11.

Referring to step 102, a computing apparatus shown in FIG. 12) first determines one or more components of the first signal. For example, referring to FIGS. 6 and 9, the computing apparatus may first determine periodic and aperiodic components, such as the periodic seasonal and aperiodic weather components 40 (FIG. 6) and 70 (FIG. 9), of a first temperature signal as described above in conjunction with FIG. 10. The computing apparatus may also correct the aperiodic component of the first signal by, e.g., filtering noise therefrom as described above in conjunction with FIG. 10.

Next, at step 104, the computing apparatus interpolates one or more portions (e.g., empty portions) of the second signal in response to the determined one or more components of the first signal. First, the computing apparatus interpolates one or more portions of one or more components of the second signal in response to the one or more components of the first signal determined at step 102. For example, the computing apparatus may interpolate one or more empty portions of a periodic component of a second temperature signal using the periodic component of a first temperature signal, and may interpolate one or more empty portions of an aperiodic component of the second temperature signal using the aperiodic component of the first temperature signal. And if the computing apparatus corrected an error in (e.g., filtered noise from) the aperiodic component of the first temperature signal at step 102, then the interpolated portions of the aperiodic component of the second temperature signal are also error corrected. Next, the computing apparatus interpolates the one or more empty portions of the second signal in response to the interpolated one or more components of the second signal. For example, the computing apparatus may interpolate the one or more empty portions of a second temperature signal by summing the periodic seasonal and aperiodic weather components of the second temperature signal, where the computing apparatus interpolated the periodic seasonal and aperiodic weather components of the second temperature signal in response to the periodic seasonal and aperiodic weather components of the first temperature signal per above.

Alternate embodiments of interpolation algorithm of FIG. 12 are contemplated. For example, the algorithm may include fewer or more steps than described, and these steps may be performed in any suitable order.

FIG. 13 is a flow diagram 110 of an embodiment of a general algorithm, which is effectively a combination of the general algorithms of FIGS. 10 and 12 above. That is, the algorithm of FIG. 13 is for interpolating one or more portions (e.g., empty portions) of a first signal, such as a first satellite-generated temperature signal, from one or more components, such as the aperiodic weather components 70 (FIG. 9), of the first signal and of a second signal, such as a second satellite-generated temperature signal; for example, the first temperature signal may correspond to a first surface region 18 (FIGS. 1-5), and the second temperature signal may correspond to a second surface region 18 that is adjacent to, or otherwise near, the first surface region. The algorithm of FIG. 13 may provide a more accurate interpolation of the first signal than either of the algorithms FIGS. 10 and 12 alone.

Referring to step 112 of the flow diagram 110 of FIG. 13, a computing apparatus (not shown in FIG. 13) first determines one or more components of the first signal. For example, referring to FIG. 9, the computing apparatus may first determine an aperiodic component, such as the aperiodic weather component 70, of a first temperature signal as described above in conjunction with FIG. 10. The computing apparatus may also correct the aperiodic component of the first signal by, e.g., filtering noise therefrom as described above in conjunction with FIG. 10.

Next, at step 114, the computing apparatus determines one or more components of the second signal. For example, referring to FIG. 9, the computing apparatus may determine an aperiodic component, such as the aperiodic weather component 70, of a second temperature signal as described above in conjunction with FIG. 10. The computing apparatus may also correct the aperiodic component of the second sigma by, e.g., filtering noise therefrom as described above in conjunction with FIG. 10.

Then, at step 116, the computing apparatus interpolates one or more portions (e.g., empty portions) of the first signal in response to the determined one or more components of the first and second signals. First, the computing apparatus interpolates one or more portions of one or more components of the first signal in response to the one or more components of the first and second signals determined at step 114. For example, the computing apparatus may interpolate one or more empty portions of an aperiodic component of a first temperature signal using the aperiodic components of the first temperature signal and of a second temperature signal, where the computing apparatus suitably weights the contributions of the non-empty portions of the aperiodic components of the first and second temperature signals. And if the computing apparatus corrected the aperiodic components of the first and second temperature signals at steps 112 and 114 by, e.g., filtering noise therefrom, then the interpolated portions of the aperiodic component of the first temperature signal are also corrected (e.g., noise filtered). Then, the computing apparatus interpolates the one or more empty portions of the first signal in response to the interpolated one or more components of the first signal. For example, the computing apparatus may interpolate the one or more empty portions of a first temperature signal by summing the periodic seasonal and aperiodic weather components of the first temperature signal, where the computing apparatus interpolated the aperiodic weather component of the first temperature signal per above.

Alternate embodiments of interpolation algorithm of FIG. 13 are contemplated. For example, the algorithm may include fewer or more steps than described, and these steps may be performed in any suitable order.

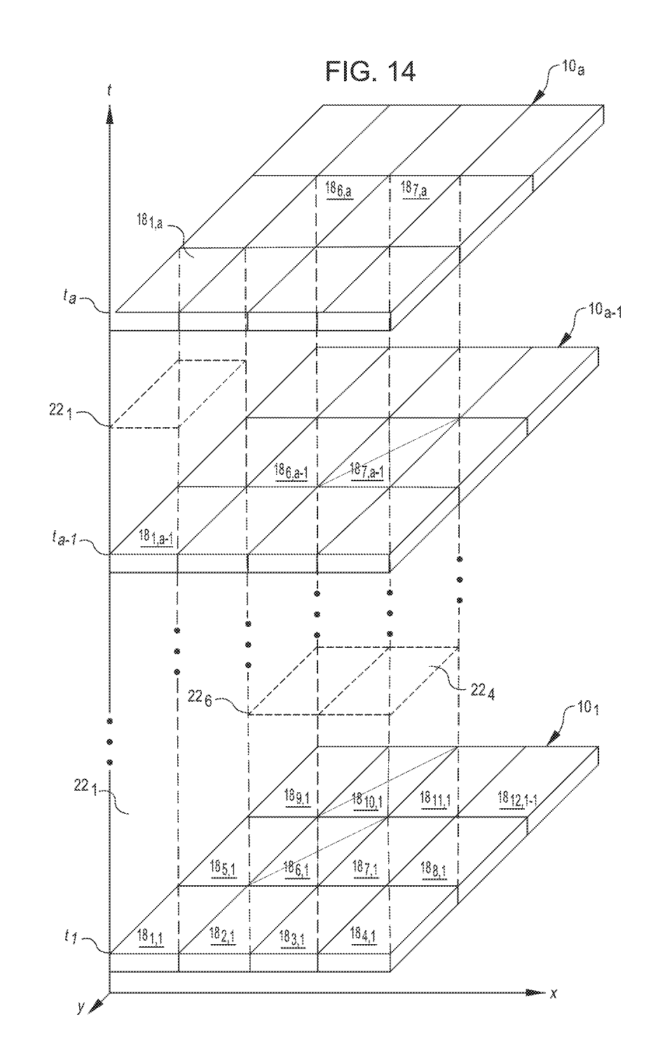

Referring to FIGS. 14-20, below is described an embodiment of an interpolation technique that incorporates at least some of the general interpolation and error-correction concepts described above in conjunction with FIGS. 10-13. For purposes of explanation, the signals having empty portions to be interpolated are formed from temperatures measured by NASA's AQUA satellite (e.g., the satellite 20 of FIGS. 1 and 3) of surface regions of the earth. Unless otherwise noted, all temperatures are in units of Kelvin (K), although it is contemplated that the below-described algorithms may be modified conventionally for compatibility with other units of temperature such as Celsius (C) or Fahrenheit (F).

FIG. 14, which is similar to FIG. 2, is a plot of the landmass section 10 of FIG. 1 versus time, with each snapshot 10₁-10ₐ of the landmass section representing a respective sample time t₁-t_{α} when NASA's AQUA satellite (e.g., the satellite 20 of FIG. 1) measures the temperatures of the surface regions 18, and where “X” indicates an empty temperature measurement for the corresponding surface region at the corresponding sample time. For purposes of explanation, it is assumed that the interval between immediately adjacent sample times I is approximately one day, and that each column 22 includes ten years of temperature measurements, i.e., each column is ten years “tall”. For further purposes of explanation, the following discussion focuses on the surface regions 18₆ and 18₇ and the ten-years worth of temperature measurements for these surface regions, where each temperature measurement corresponds to a respective one of the snapshots 18_{6,1}-18_{6,a} and 18_{7,1}-18₇ of these surface regions.

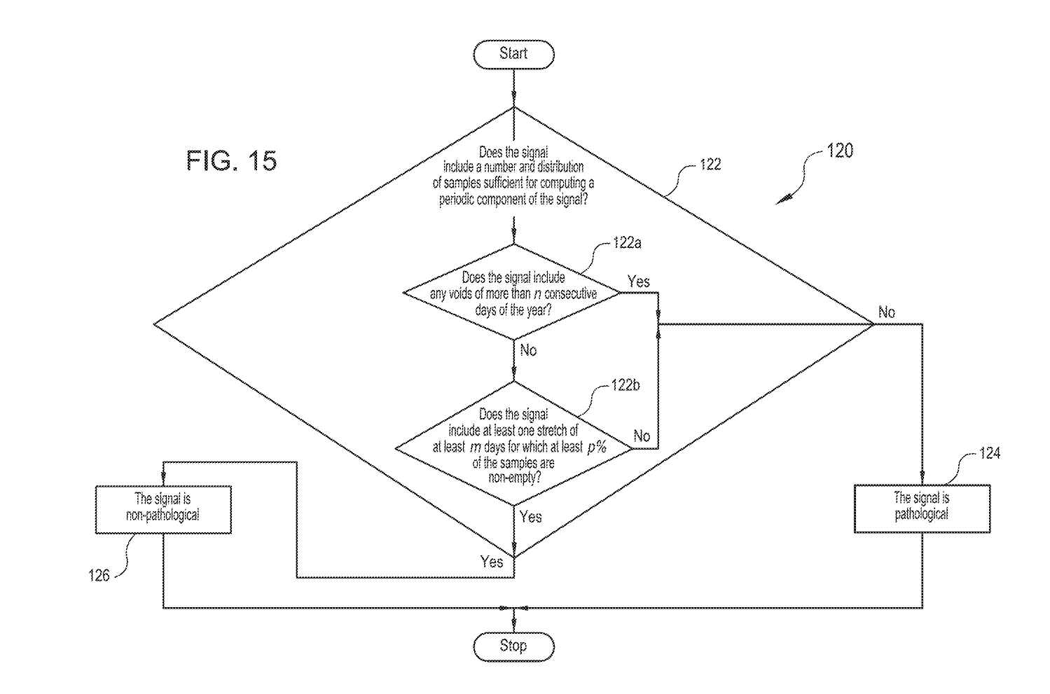

FIG. 15 is a flow diagram 120 of an embodiment of an algorithm for determining what interpolation algorithm to use for interpolating empty portions of a signal. For example, if a computing apparatus (not shown in FIG. 15) determines that the signal meets a criterion, then the computing apparatus may use an interpolation algorithm that determines one or more components of the signal; but if the computing apparatus determines that the signal does not meet the criterion, then the computing apparatus may us an interpolation algorithm that does not determine a component of the signal.

Referring to step 122, the computing apparatus first determines whether the temperature signal associated with a surface region 18 (FIG. 14) includes a number and distribution of samples sufficient for computing a periodic component (e.g., a periodic seasonal component) of the signal.

At substep 122a, according to a first prong of this test, the computing apparatus determines whether the temperature signal has any “voids” of more than n consecutive days of the year with empty temperature measurements.

If the signal does not include any such void, then the signal passes this prong of the test, and the computing apparatus proceeds to substep 122b.

But if the signal includes such a void, then the signal “fails” the test, and the computing apparatus proceeds to step 124, where the computing apparatus labels the temperature signal a “pathological” signal (or, alternatively, labels the surface region corresponding to the temperature signal a “pathological” surface region or “pixel”), which means that, as should become more evident below, the standard deviation at the central point of an n+1 day-of-year (DOY)-wide data gap, i.e., calculation window, cannot be determined for all samples of the temperature signal. More specifically, a DOY index is a specific date with respect to January 1^{st} of the same year. For example, 3 Feb. 2002 and 3 Feb. 2007 are both DOY=34. If the temperature signal covers thirty years, then for each the temperature signal would have a maximum of thirty non-empty temperature measurements (except for leap days), one temperature measurement for each covered year. Furthermore, assume, for example, that n=30. If the temperature signal includes no non-empty temperature measurements in the month of January (thirty one consecutive days) over the entire thirty years, then the temperature signal does not pass the first prong of the test because the signal has more (thirty one days in this example) than n=30 consecutive days of the year with empty temperature measurements. However, if the temperature signal includes just one non-empty temperature measurement in any one of the thirty Januarys covered by the temperature signal, then the temperature signal passes the first prong of the test because the signal has no more than n=30 consecutive days of the year with empty temperature measurements (assuming that the temperature signal does not elsewhere include a void of at least thirty one days). Although n=30 is used for example purposes, n may have any value that is suitable for the application of the interpolation technique.

At substep 122b, according to a second prong of the test, the computing apparatus determines whether the temperature signal includes at least one stretch of in days for which at least p % of the samples are non-empty, regardless of whether these samples are consecutive. If the temperature signal includes such a stretch of m days, then the signal passes the second prong of the test, and the computing apparatus proceeds to step 126, at which step the computing apparatus labels the signal a non-pathological signal. But if the temperature signal does not include such a stretch of in days, then the signal fails the second prong of the test, and the computing apparatus proceeds to step 124, at which step the computing apparatus labels the signal a pathological signal. For example, if m=366 days, p %=10%, and the signal covers a period of thirty years and includes just four non-empty temperature measurements per month over just one year of the thirty years, then the signal would pass the second prong of the test because 48 days (in a 366-day period)>[10%·(366 days)=36.6 days in a 366-day period]. Although m=366 is used for example purposes (366 is the number of days in a leap year), m may have any value that is suitable for the application.

Referring to FIGS. 14 and 15, alternate embodiments of the algorithm are contemplated. For example, instead of the algorithm of FIG. 15, a computing apparatus may use another algorithm that is suitable for determining which of multiple interpolation techniques to use for interpolating a signal such as a surface-region temperature signal.

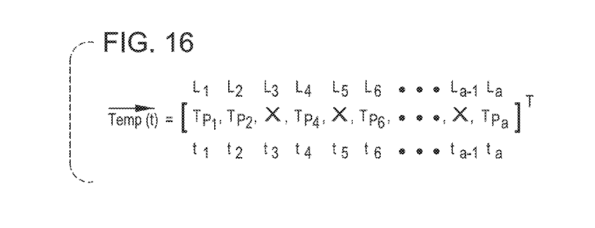

Referring to FIGS. 16-20, in the following discussion, unless otherwise noted, it is further assumed, for example purposes, that the temperature signal associated with the surface region 18₆ (FIG. 14) is a non-pathological signal, and that the temperature signal associated with the surface region 18₇ (FIG. 14) is a pathological signal.

FIG. 16 is a diagram of a temperature signal Temp(t) corresponding to the surface region 18₆ of FIG. 1 according to an embodiment. Temp(t) is a time-domain signal (i.e., a signal that is a function of time), is shown in column-vector form (“T” indicates a vector transpose), and includes measured temperature values (e.g., measured by the satellite 20 of FIGS. 1 and 3) Tp₁, Tp₂, . . . , Tp_{α} in respective vector-element locations L₁, L₂, . . . , L_{α}, which locations respectively correspond to sample times t₁, t₂, . . . , t_{α}. That is, Tp₁ is the temperature of the snapshot 18_{6,1} (FIG. 14) of the surface region 18₆ measured at sample time t₁, Tp₂ is the temperature of the snapshot 18_{6,2} of the surface region 18₆ at sample time t₂, and so on. And “X” indicates a vector-element location L that is empty, i.e., that holds no usable temperature value Tp. That is, in this example, at least the vector locations L₃, L₅, and L_{α-1} are empty, and thus constitute empty portions of Temp(t). Although not shown, Temp(t) may also include consecutive empty locations L. Furthermore, because Temp(t) is a column vector in this example, it is referred to a vector symbol [symbol] hereinafter.

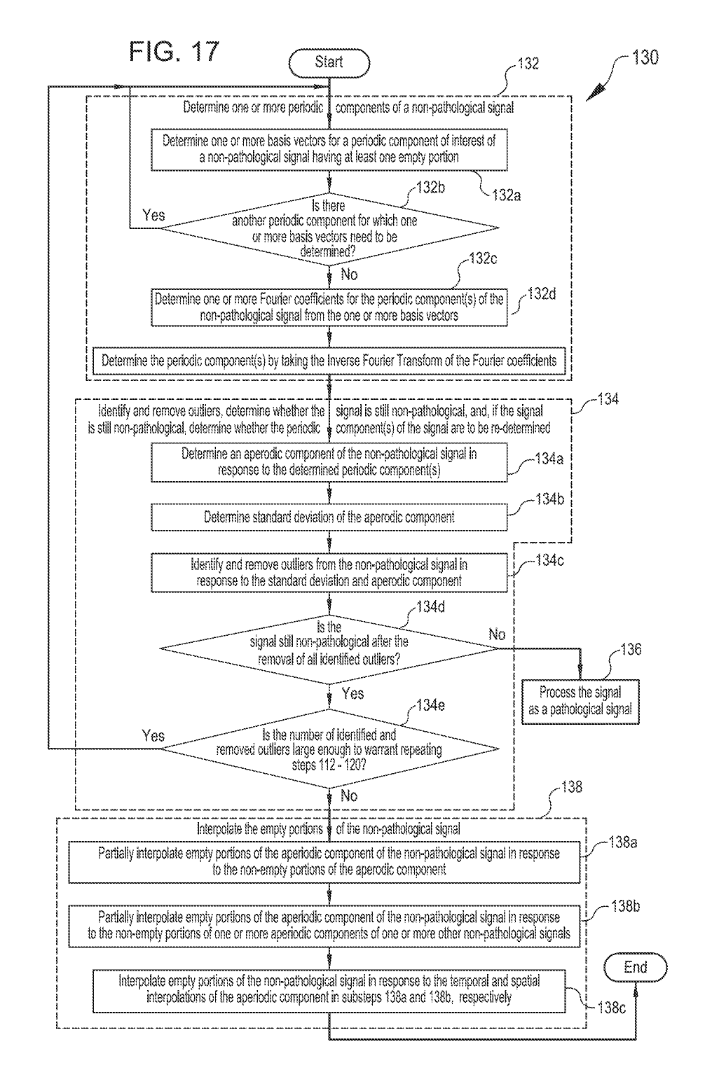

FIG. 17 is a flow diagram 130 of an algorithm for interpolating one or more empty portions of a non-pathological signal (e.g., a signal having a number and distribution of samples sufficient to allow a determination of at least one component of the signal for a particular application) according to an embodiment. For example purposes, the algorithm is discussed in conjunction with interpolating one or more empty portions of the non-pathological temperature signal [symbol] , although it is contemplated that the algorithm may be used to interpolate one or more empty portions of a signal other than [symbol] .

At step 132 of FIG. 17, a computing apparatus (not shown in FIG. 17) first determines one or more periodic components of the non-pathological signal [symbol] .

At substep 132a, the computing apparatus determines one or more Discrete Fourier Transform (DFT) basis vectors for a first periodic component, such as a periodic seasonal component, of the signal [symbol] . For example purposes, it is assumed that the computing apparatus determines, as the first periodic component, a periodic seasonal component \vec{S(t)} of the signal [symbol] , where \vec{S(t)} has a fundamental period T=365.2475 days (i.e., one solar year).

Still at substep 132a, the computing apparatus first determines a basis function V₀, which is the 0^{th} harmonic (i.e., the 0^{th} or DC level) of the Discrete Fourier Transform (DFT) of the periodic seasonal component \vec{S(t)} (and of any subsequently determined periodic components, such as the periodic noise components discussed below), according to the following equation:

$${V}_{0} = \frac{1}{\sqrt{N}} \qquad ( 2 )$$

where N is the number of valid, i.e., non-empty, samples Tp in [symbol] (FIG. 16). For example, if the signal [symbol] covers a period of α=365 days, but includes ten empty locations L (i.e., ten locations L marked with “X”), then N=365−10=355.

Next, the computing apparatus determines complete basis functions for a number hs of the non-zero DFT harmonics of the periodic seasonal component \vec{S(t)} according to the following equations, where hs is a function of the application in which the interpolated version of [symbol] is to be used:

$$\begin{matrix}{V}_{2 i - 1} = \frac{1}{\sqrt{{C}_{2 i - 1}}} cos ( \frac{2 i π}{T} t ) & ( 3 ) \\ {V}_{2 i} = \frac{1}{\sqrt{{C}_{2 i}}} sin ( \frac{2 i π}{T} t ) & ( 4 )\end{matrix}$$

where i=1, . . . , hs, C₂ᵢ₋₁ and C₂ᵢ are normalization constants, the calculation of which is discussed below, T is the fundamental period, i.e., the period of the first harmonic i=1, and the computing apparatus determines two complete basis functions for each non-zero harmonic i, one for the cosine transform and one for the sine transform. In an example application, where [symbol] includes temperatures Tp measured by NASA's AQUA satellite, T=365.2475 days, and only the first hs=6 non-zero DFT harmonics are used to generate the periodic seasonal component \vec{S(t)}; therefore, the computing apparatus will determine the complete basis functions V₁-V₁₂ for the non-zero first through sixth harmonics (i.e., hs=6 and, therefore, i=1, . . . , hs=6) of \vec{S(t)} according to the following equations:

$$\begin{matrix}{V}_{1} = \frac{1}{\sqrt{{C}_{1}}} cos ( \frac{2 π}{365.2475} t ) & ( 5 ) \\ {V}_{2} = \frac{1}{\sqrt{{C}_{2}}} sin ( \frac{2 π}{365.2475} t ) & ( 6 ) \\ {V}_{3} = \frac{1}{\sqrt{{C}_{3}}} cos ( \frac{4 π}{365.2475} t ) & ( 7 ) \\ {V}_{4} = \frac{1}{\sqrt{{C}_{4}}} sin ( \frac{4 π}{365.2475} t ) & ( 8 ) \\ {V}_{5} = \frac{1}{\sqrt{{C}_{5}}} cos ( \frac{6 π}{365.2475} t ) & ( 9 ) \\ {V}_{6} = \frac{1}{\sqrt{{C}_{6}}} sin ( \frac{6 π}{365.2475} t ) & ( 10 ) \\ {V}_{7} = \frac{1}{\sqrt{{C}_{7}}} cos ( \frac{8 π}{365.2475} t ) & ( 11 ) \\ {V}_{8} = \frac{1}{\sqrt{{C}_{8}}} sin ( \frac{8 π}{365.2475} t ) & ( 12 ) \\ {V}_{9} = \frac{1}{\sqrt{{C}_{9}}} cos ( \frac{10 π}{365.2475} t ) & ( 13 ) \\ {V}_{10} = \frac{1}{\sqrt{{C}_{10}}} sin ( \frac{10 π}{365.2475} t ) & ( 14 ) \\ {V}_{11} = \frac{1}{\sqrt{{C}_{11}}} cos ( \frac{12 π}{365.2475} t ) & ( 15 ) \\ {V}_{12} = \frac{1}{\sqrt{{C}_{12}}} sin ( \frac{12 π}{365.2475} t ) & ( 16 )\end{matrix}$$

Then, still at substep 132a, the computing apparatus generates a complete basis vector \vec{V₀}, and generates partial basis vectors \vec{V₂ₜ₋₁}, and \vec{V₂ₜ} from the respective basis functions in equations (5)-(16) above. The computer apparatus generates the partial basis vectors \vec{V₂ₜ₋₁} and \vec{V₂ₜ}, for i=1, . . . , hs, from the corresponding basis functions V₂ᵢ₋₁ and V₂ᵢ by substituting for “t” in each basis function the respective sample times t₁-t_{α} corresponding to the locations L₁-L_{α} of [symbol] (FIG. 16); consequently, each partial basis vector \vec{V₂ₜ₋₁} and \vec{V₂ₜ₋₁} will have α elements. For example, if the interval between consecutive samples times t₁-t_{α} is one day, and there are α=365 sample times t₁-t₃₆₅, and thus α=365 elements in [symbol] , then the computing apparatus substitutes 1, 2, . . . , 365 days for “t” in each basis function to generate the respective α elements of each of the partial basis vectors \vec{V₂ₜ₋₁} and \vec{V₂ₜ₋₁}. But because \vec{V₀} includes no cosine or sine function, or any other function, of t, all of the α elements of \vec{V₀} equal

$$\frac{1}{\sqrt{N}} ,$$

and no further action is required to complete the basis vector \vec{V₀}.

Next, the computing apparatus generates the normalization constants C₂ᵢ₋₁ such that:

\vec{V₂ₜ₋₁}·\vec{V₂ₜ₋₁}=1 (17)

where the dot-product operator “·” sums only over the elements of the partial basis vector \vec{V₂ₜ₋₁} that correspond to the non-empty locations L of [symbol] . For example, referring to FIG. 16, at least the locations L₃, L₅, and L_{α-1} of [symbol] are empty; therefore, the dot-product operator “·” does not include in its resulting sum any terms that include elements from the corresponding locations L₃, L₅, and L_{α-1} (and from any other locations L corresponding to empty portions of [symbol] ) of the partial basis vector \vec{V₂ₜ₋₁}. The computing apparatus effectively transforms the partial basis vectors \vec{V₂ₜ₋₁} into complete basis vectors \vec{V₂ₜ₋₁} by multiplying the elements of each partial basis vector \vec{V₂ₜ₋₁} by the respective value of

$$\frac{1}{\sqrt{{C}_{2 i - 1}}}$$

per equation (3) above.

Similarly, still at substep 132a, the computing apparatus also generates the normalization constants C₂ᵢ such that:

\vec{V₂ₜ}·\vec{V₂ₜ}=1 (18)

where the dot-product operator “·” sums only over the elements of the partial basis vector \vec{V₂ₜ} that correspond to the non-empty locations L of [symbol] ). The computing apparatus effectively transforms the partial basis vectors \vec{V₂ₜ} into completed basis vectors \vec{V₂ₜ} by multiplying the elements of each partial basis vectors \vec{V₂ₜ} by the respective value of

$$\frac{1}{\sqrt{{C}_{2 i}}}$$

per equation (4) above.

Then, at substep 132b of FIG. 17, the computing apparatus determines whether one or more basis vectors for another periodic component of the signal [symbol] are to be determined.

If there are no more periodic components of the signal [symbol] for which basis vectors are to be determined, then the computing apparatus proceeds to step 134.

But if there is one or more other periodic components of the signal [symbol] for which basis vectors are to be determined, then the computing apparatus returns to substep 132a.

For example, suppose that the computing apparatus is to determine basis vectors for three additional periodic components, periodic noise components [symbol] , [symbol] , and [symbol] of [symbol] , where these noise components respectively have fundamental periods T₁=16 days, T₂=4096/1781 days, and T₃=4096/1803 days (these are fundamental noise periods of temperature measurements made by NASA's AQUA satellite per above).

To determine basis vectors for [symbol] , [symbol] , and [symbol] , the computing apparatus repeats substep 132a three times, once for each of these periodic noise components.

Referring again to substep 132a, for the periodic first noise component [symbol] , the computing apparatus generates basis functions for an arbitrary number hn₁ of the non-zero DFT harmonics of [symbol] according to equations (3) and (4) above, where the number hn₁ is a function of the application in which the interpolated version of [symbol] is to be used, and i=hs+1, . . . , hs+hn₁. Note that the harmonic index i does not start at zero, but starts at the next integer after hs, which is the number of harmonics of the periodic seasonal component \vec{S(t)} for which basis vectors \vec{V₂ₜ₋₁}, and \vec{V₂ₜ} were previously calculated. In an example, the signal [symbol] includes temperatures Tp measured by NASA's AQUA satellite, T=16 days for the periodic first noise component [symbol] , and only the first hn₁=8 non-zero DFT harmonics (up to the Nyquist frequency 8/16=½) of the fundamental frequency F=1/T= 1/16 are used to generate the periodic first noise component [symbol] . Therefore, where hs=6 for the periodic seasonal component \vec{S(t)} per the above example, the computing apparatus will determine the basis functions V₁₃-V₂₈ for the non-zero first through eighth harmonics (Le., hn₁=8 and i=6+1=7, . . . , 6+8=14) of [symbol] according to the following equations:

$$\begin{matrix}{V}_{13} = \frac{1}{\sqrt{{C}_{13}}} cos ( \frac{2 π}{16} t ) & ( 19 ) \\ {V}_{14} = \frac{1}{\sqrt{{C}_{14}}} sin ( \frac{2 π}{16} t ) & ( 20 ) \\ {V}_{15} = \frac{1}{\sqrt{{C}_{15}}} cos ( \frac{4 π}{16} t ) & ( 21 ) \\ {V}_{16} = \frac{1}{\sqrt{{C}_{16}}} sin ( \frac{4 π}{16} t ) & ( 22 ) \\ {V}_{17} = \frac{1}{\sqrt{{C}_{17}}} cos ( \frac{6 π}{16} t ) & ( 23 ) \\ {V}_{18} = \frac{1}{\sqrt{{C}_{18}}} sin ( \frac{6 π}{16} t ) & ( 24 ) \\ {V}_{19} = \frac{1}{\sqrt{{C}_{19}}} cos ( \frac{8 π}{16} t ) & ( 25 ) \\ {V}_{20} = \frac{1}{\sqrt{{C}_{20}}} sin ( \frac{8 π}{16} t ) & ( 26 ) \\ {V}_{21} = \frac{1}{\sqrt{{C}_{21}}} cos ( \frac{10 π}{16} t ) & ( 27 ) \\ {V}_{22} = \frac{1}{\sqrt{{C}_{22}}} sin ( \frac{10 π}{16} t ) & ( 28 ) \\ {V}_{23} = \frac{1}{\sqrt{{C}_{23}}} cos ( \frac{12 π}{16} t ) & ( 29 ) \\ {V}_{24} = \frac{1}{\sqrt{{C}_{24}}} sin ( \frac{12 π}{16} t ) & ( 30 ) \\ {V}_{25} = \frac{1}{\sqrt{{C}_{25}}} cos ( \frac{14 π}{16} t ) & ( 31 ) \\ {V}_{26} = \frac{1}{\sqrt{{C}_{26}}} sin ( \frac{14 π}{16} t ) & ( 32 ) \\ {V}_{27} = \frac{1}{\sqrt{{C}_{27}}} cos ( \frac{16 π}{16} t ) & ( 33 ) \\ {V}_{28} = \frac{1}{\sqrt{{C}_{28}}} sin ( \frac{16 π}{16} t ) & ( 34 )\end{matrix}$$

But because

$$sin ( \frac{16 π}{16} t ) = sin π t = 0 ,$$

the basis function V₂₈=0 of equation (34) may, as described below, be omitted after the computing apparatus generates complete basis vectors for all other periodic components of [symbol] to be determined.

Next, still a substep 132a, the computing apparatus generates partial basis vectors \vec{V₂ₜ₋₁}, and \vec{V₂ₜ} from the respective basis functions in equations (19)-(34) above for i=hs+1, . . . , hs+hn₁ by substituting for “t” in each basis function the respective sample times t₁-t_{α} corresponding to the locations L₁-L_{α} of [symbol] (FIG. 14) as discussed above, such that each partial basis vector \vec{V₂ₜ₋₁} and \vec{V₂ₜ} has α elements.

Then, the computing apparatus generates the normalization constants C₂ᵢ₋₁ for i=hs+1=7, . . . , hs+hn₁=14 such that:

\vec{V₂ₜ₋₁}·\vec{V₂ₜ₋₁}=1 (35)

where the dot-product operator “·” sums only over the elements of the partial basis vectors \vec{V₂ₜ₋₁} that correspond to the non-empty locations L of [symbol] (FIG. 14) as discussed above. The computing apparatus effectively converts the partial basis vectors \vec{V₂ₜ₋₁} into complete basis vectors \vec{V₂ₜ₋₁} by multiplying the elements of each partial basis vector \vec{V₂ₜ₋₁} by the respective value of

$$\frac{1}{\sqrt{{C}_{2 i - 1}}}$$

per equation (3) above.

Similarly, the computing apparatus also generates the normalization constants C₂ᵢ for i=hs+1=7, . . . , hs+hn₁=14 such that:

\vec{V₂ₜ}·\vec{V₂ₜ}=1 (36)

where the dot-product operator “·” sums only over the elements of the partial basis vectors \vec{V₂ₜ} that correspond to the non-empty locations L of [symbol] . The computing apparatus effectively converts the partial basis vectors \vec{V₂ₜ} into complete basis vectors \vec{V₂ₜ} by multiplying the elements of each partial basis vector \vec{V₂ₜ} by the respective values of

$$\frac{1}{\sqrt{{C}_{2 i}}}$$

per equation (4) above.

Sill at substep 132a, for the periodic second noise component [symbol] , the computing apparatus generates basis functions for an arbitrary number hn₂ of the non-zero DFT harmonics of [symbol] according to equations (3) and (4) above, where the number hn₂ is a function of the application in which the interpolated version of [symbol] is to be used, and i=h+hn₁+1, . . . , h+hn₁+hn₂. Note that the index i does not start at zero, but starts at the next integer after h+hn₁, which sum is the total number of harmonics of the periodic seasonal component \vec{S(t)} and the periodic first noise component [symbol] for which the computing apparatus previously calculated basis functions V and basis vectors \vec{V}. In an example, [symbol] includes temperatures Tp measured by NASA's AQUA satellite, T=4096/1781 days for the periodic second noise component [symbol] , and basis functions for only the first hn₂=1 non-zero DFT harmonic of the fundamental frequency F=1781/4096 are used to generate the periodic second noise component [symbol] . Therefore, where hs=6 for the periodic seasonal component \vec{S(t)} and hn₁=8 for the periodic first noise component [symbol] per the above examples, the computing apparatus determines the basis functions V₂₉-V₃₀ for the non-zero first harmonic (i.e., hn₂=1 and i=6+8+1=15) of [symbol] according to the following equations:

$$\begin{matrix}{V}_{29} = \frac{1}{\sqrt{{C}_{29}}} cos ( \frac{2 π 1781}{4096} t ) & ( 37 ) \\ {V}_{30} = \frac{1}{\sqrt{{C}_{30}}} sin ( \frac{2 π 1781}{4096} t ) & ( 38 )\end{matrix}$$

Then, the computing apparatus generates partial basis vectors \vec{V₂ₜ₋₁}=\vec{V₂₉} and \vec{V₂ₜ}=\vec{V₃₀} from the respective basis functions in equations (37)-(38) above for i=h+hn₁+1=15 by substituting for “t” in each basis function the respective sample times t₁-t_{α} corresponding to the locations L₁-L_{α} of [symbol] (FIG. 14) as discussed above such that each partial basis vector \vec{V₂₉} and \vec{V₃₀} has α elements.

Next, the computing apparatus next generates the normalization constant C₂ₜ₋₁=C₂₉ for i=h+hn₁+1=15 such that:

\vec{V₂₉}·\vec{V₂₉}=1 (39)

where the dot-product operator “·” sums only over the elements of the partial basis vector \vec{V₂₉} that correspond to the non-empty locations L of [symbol] as discussed above. The computing apparatus effectively converts the partial basis vector \vec{V₂₉} into a complete basis vector \vec{V₂₉} by multiplying the elements of the partial basis vector \vec{V₂₉} by the value of

$$\frac{1}{\sqrt{{C}_{29}}}$$

per equation (3) above.

Similarly, the computing apparatus also generates the normalization constant C₂ᵢ=C₃₀ for i=h+hn₁+1=15 such that:

\vec{V₃₀}·\vec{V₃₀}=1 (40)

where the dot-product operator “·” sums only over the elements of the partial basis vector \vec{V₃₀} that correspond to the non-empty locations L of [symbol] . The computing apparatus effectively converts the partial basis vector \vec{V₃₀} into a completed basis vector \vec{V₃₀} by multiplying the elements of the partial basis vector \vec{V₃₀} by the value of

$$\frac{1}{\sqrt{{C}_{30}}}$$

per equation (4) above.

And, still at substep 132a, for the periodic third noise component [symbol] , the computing apparatus generates basis functions for an arbitrary number hn₃ of the non-zero DFT harmonics of [symbol] according to equations (3) and (4) above, where the number hn₃ is a function of the application in which the interpolated version of [symbol] is to be used, and i=h+hn₁+hn₂+1, . . . , h+hn₁+hn₂+hn₃. Note that the index i does not start at zero, but starts at the next integer after h+hn₁+hn₂, which sum is the total number of harmonics of the periodic seasonal component \vec{S(t)}, the periodic first noise component [symbol] , and the periodic second noise component [symbol] for which the computing apparatus previously calculated basis functions V and basis vectors \vec{V}. In an example, [symbol] includes temperatures Tp measured by NASA's AQUA satellite, T=4096/1803 days for the periodic third noise component [symbol] , and basis functions for only the first hn₃=1 non-zero DFT harmonic are needed to generate the periodic third noise component [symbol] . Therefore, where hs=6 for the periodic seasonal component \vec{S(t)}, hn₁=8 for the periodic first noise component [symbol] , and hn₂=1 for the periodic second noise component [symbol] per the above examples, the computing apparatus determines the basis functions V₃₁-V₃₂ for the non-zero first harmonic (i.e., hn₃=1 and i=6+8+1+1=16) of [symbol] according to the following equations:

$$\begin{matrix}{V}_{31} = \frac{1}{\sqrt{{C}_{31}}} cos ( \frac{2 π 1803}{4096} t ) & ( 41 ) \\ {V}_{32} = \frac{1}{\sqrt{{C}_{32}}} sin ( \frac{2 π 1803}{4096} t ) & ( 42 )\end{matrix}$$

Next, the computing apparatus generates partial basis vectors \vec{V₂ₜ₋₁}=\vec{V₃₁}, and \vec{V₂ₜ}=\vec{V₃₂} from the respective basis functions in equations (41)-(42) above for i=h+hn₁+hn₂+1=16 by substituting for “t” in each basis function the respective sample times t₁-t_{α} corresponding to the locations L₁-L_{α} of [symbol] (FIG. 14) as discussed above, such that each partial basis vector \vec{V₃₁} and \vec{V₃₂} has α elements.

Then, the computing apparatus generates the normalization constant C₂ᵢ₋₁=C₃₁ for i=hs+hn₁+hn₂+1=16 such that:

\vec{V₃₁}·\vec{V₃₁}=1 (43)

where the dot-product operator “·” sums only over the elements of \vec{V₃₁} that correspond to the non-empty locations L of [symbol] as discussed above. The computing apparatus effectively converts the partial basis vector \vec{V₃₁} into a complete basis vector \vec{V₃₁} by multiplying the elements of the partial basis vector \vec{V₃₁} by the value of

$$\frac{1}{\sqrt{{C}_{31}}}$$

per equation (3) above.

Similarly, the computing apparatus also generates the normalization constant C₂₁=C₃₂ for i=h+hn₁+hn₂+1=16 such that:

\vec{V₃₂}·\vec{V₃₂}=1 (44)

where the dot-product operator “·” sums only over the elements of \vec{V₃₂} that correspond to the non-empty locations L of [symbol] . The computing apparatus effectively converts the partial basis vector \vec{V₃₂} into a complete basis vector \vec{V₃₂} by multiplying the elements of the partial basis vector \vec{V₃₂} by the value of

$$\frac{1}{\sqrt{{C}_{32}}}$$

per equation (4) above.

Still referring to FIG. 17, at substep 132c of step 132, the computing apparatus determines the Fourier coefficients for the harmonics of all of the periodic components of [symbol] for which the computing apparatus determined complete basis vectors per substeps 132a-132b as described above.

Prior to substep 132c, one may visualize that the computing apparatus generates two conceptual columns. Here, “conceptual” indicates that in actuality, the computing apparatus may not generate physical/displayable columns, but may instead generate a representation of the columns, or may otherwise operate in a manner that is consistent with the use of the columns; hereinafter, such a column, or any other item described as being computer generated, may be a conceptual item even if not expressly stated. In the first column are all of the complete basis vectors \vec{V₂ₜ} and \vec{V₂ₜ₋₁}, and the second column is empty. For clarity of notation, the complete basis vectors \vec{V₂ₜ} and \vec{V₂ₜ₋₁} rewritten as \vec{Vₚ}, where the index p=1, 2, . . . , 2max_i, where max_i is the maximum value of i. In the above example, max_i=16 such that 2max_i=32, which is the highest index of the example basis functions V₂ᵢ and V₂ᵢ₋₁ and the corresponding complete basis vectors \vec{V₂ₜ} and \vec{V₂ₜ₋₁}.

Furthermore, for purposes of example, the embodiment of the algorithm described by the flow diagram 130 of FIG. 17 will continue to be described in conjunction with the above example, where the non-pathological signal [symbol] includes the periodic seasonal component \vec{S(t)} and the periodic noise components [symbol] , [symbol] , and [symbol] . Because, as discussed above, in this example the basis function V₂₈=0, this basis function and its corresponding basis vector \vec{V₂₈} are omitted, and the above example basis functions V having the higher indices 29-32 each have their indices reduced by one, such that there are thirty one, not thirty two, basis functions V and complete basis vectors \vec{Vₚ}. That is, V₂₉ and \vec{V₂₉} respectively become V₂₈ and \vec{V₂₈}, V₃₀ and \vec{V₃₀} respectively become V₂₉ and \vec{V₂₉}, V₃₁ and \vec{V₃₁} respectively become V₃₀ and \vec{V₃₀}, and V³² and \vec{V₃₂} respectively become V₃₁ and \vec{V₃₁}. Also recall that although the complete basis vectors \vec{Vₚ} and the temperature signal [symbol] each have the same number α of elements, the vectors \vec{Vₚ} have no empty elements, and [symbol] , at least in the described example, has at least one empty element (e.g., empty elements in at least the locations L₃, L₅, and L_{α-1} marked with “X” in FIG. 16).

Referring again to substep 132c, the computing apparatus calculates a first Fourier coefficient b₀, which is the DC or zero-frequency coefficient, according to the following equation:

b₀=\vec{V₀}· [symbol] (45)

where the dot-product operator “·” sums only over the non-empty elements of [symbol] and the corresponding elements of \vec{V₀}. For example, referring to FIG. 16, because the elements in locations L₃, L₅, and L_{α-1} of [symbol] are empty, the dot-product operator does not sum over at least the elements in locations L₃, L₅, and L_{α-1} of \vec{V₀} and [symbol] .

Next, the computing apparatus effectively removes \vec{V₀} from the first column, and places both b₀ and \vec{V₀} in the second column.

Then, the computing apparatus orthogonalizes all of the remaining complete basis vectors \vec{Vₚ} (i.e., all of the vectors \vec{Vₚ} other than \vec{V₀}) with respect to \vec{V₀} according to the following equation:

$$\vec{{V}_{p}^{′}} = \frac{\vec{{V}_{p}} - ( \vec{{V}_{p}} \cdot \vec{{V}_{0}} ) \vec{{V}_{0}}}{‖ \vec{{V}_{p}} - ( \vec{{V}_{p}} \cdot \vec{{V}_{0}} ) \vec{{V}_{0}} ‖} \qquad ( 46 )$$

where \vec{V′ₚ} is the orthogonalized version of \vec{Vₚ}, “∥ ∥” is the normal operator that takes the conventional L2 norm of a vector (the L2 norm is a scalar), and the dot-product operator “·” sums over all the elements of \vec{V₀} and \vec{Vₒ} (p ≠ 0) because these complete basis vectors have no empty portions. The computing apparatus orthogonalizes all of the basis vectors \vec{Vₚ} with respect to \vec{V₀} because the computing apparatus is attempting to generate orthogonal periodic components \vec{S(t)}, [symbol] , [symbol] , and [symbol] of [symbol] . In this context, “orthogonal” means that \vec{S(t)}+ [symbol] + [symbol] + [symbol] = [symbol] . But because [symbol] has empty portions, and \vec{S(t)}, [symbol] , [symbol] , and [symbol] are being derived from this incomplete version of [symbol] , the orthogonality between \vec{S(t)}, [symbol] , [symbol] , and [symbol] may be lost, and, therefore, errors may be introduced into the resulting Fourier components unless the complete basis vectors are orthogonalorthogonalized with respect to one another.

Next, still at substep 132c, the computing apparatus determines potential orthogonalorthogonalized Fourier coefficients b′ₚ according to the following equation:

b′ₚ=\vec{′ₚ}·\vec{Temp(t)} (47)

where p=1, . . . , 2max_i (or, per the above example where V₂₈ and \vec{V₂₈} are omitted; p=1, . . . , 2max_i-1), and the dot-product operator “·” sums only over the non-empty elements of [symbol] and the corresponding elements of \vec{V′ₚ}.

Then, the computing apparatus determines which Fourier coefficient b′ₚ has the greatest magnitude, stores this largest coefficient b′ₚ, along with its corresponding vector \vec{V′ₚ}, in the second column, and removes this vector \vec{V′ₚ} from the first column—for clarity, after the computing apparatus effectively removes this vector \vec{V′ₚ} from the first column and moves it into the second column, the computing apparatus can rename it \vec{V′ₚₛ}. The computing apparatus also effectively saves all of the remaining (i.e. all but the largest) Fourier coefficients b′ₚ in the first column.

Next, the computing apparatus determines whether there are, in the first column, any more orthogonalized complete basis vectors \vec{V′ₚ}, which were orthogonalorthogonalized with respect to \vec{Vₒ} per equation (46 above). If there are no more orthogonalorthogonalized complete basis vectors \vec{V′ₚ} in the first column, then the computing apparatus proceeds to substep 132d. But if there are more orthogonalorthogonalized completed basis vectors \vec{V′ₚ} in the first column, then the computing apparatus continues to determine Fourier coefficients as described immediately below.

Still at subset 132c, if there are still more orthogonalorthogonalized complete basis vectors \vec{V′ₚ} in the first column, then the computing apparatus further orthogonalizes these vectors \vec{V′ₚ} remaining in the first column with respect to \vec{V′ₚₛ} according to the following equation:

$$\vec{{V}_{p}^{″}} = \frac{\vec{{V}_{p}^{′}} - ( \vec{{V}_{p}^{′}} \cdot \vec{{V}_{ps}^{′}} ) \vec{{V}_{ps}^{′}}}{‖ \vec{{V}_{p}^{′}} - ( \vec{{V}_{p}^{′}} \cdot \vec{{V}_{ps}^{′}} ) \vec{{V}_{ps}^{′}} ‖} \qquad ( 48 )$$

where \vec{V″ₚ} is the further orthogonalized version of \vec{V′ₚ},

Then, the computing apparatus determines whether there are more Fourier coefficients b to be calculated. For example, the computing apparatus may determine that there are no more Fourier coefficients to be calculated if at least one of the following is true: (1) the largest potential Fourier coefficient bₚ^{(k)} (k is the number of prime symbols “′”) remaining in the first column is less than an arbitrarily selected tolerance threshold, and (2) the quantity ∥\vec{V′ₚ}−(\vec{V′ₚ}·\vec{V′ₚₛ})\vec{V′ₚₛ}∥ is less than an arbitrarily selected tolerance threshold (this tolerance threshold may be different than the threshold in clause (1)). Either of these conditions (1) and (2) being true indicates that the contribution of the harmonics for which respective Fourier coefficients bₚ^{(k)} have not already been calculated and loaded into the second column are negligible for the particular application.

If the computing apparatus determines that there are no more Fourier coefficients bₚ^{(k)} to calculate, then it proceeds to step 132d. But if the computing apparatus determines that there are more Fourier coefficients bₚ^{(k)} to calculate, then it continues, according to step 132c per above, to calculate potential Fourier coefficients bₚ^{(k)} using the orthogonalorthogonalized basis vectors \vec{Vₚ^{(k)}} remaining in first column per equation (47), to save the largest of these coefficients in the second column along with the basis vector \vec{Vₚ^{(k)}} that generated it, to orthogonalize further, per equation (48), the basis vectors \vec{Vₚ^{(k)}} remaining in the first column with respect to the basis vector that generated the largest Fourier coefficient bₚ^{(k)}, and to repeat this procedure until it determines that there are no more Fourier coefficients bₚ^{(k)} to calculate.

At substep 132d, the computing apparatus generates the time-domain periodic component(s) of the signal (e.g., [symbol] ) to he interpolated by taking the Inverse Discrete Fourier Transform (IDFT) of the respective pairs of Fourier coefficients b in the second column over all sample times t in the signal regardless of whether the signal is empty at one or more sample times t.

Still at substep 132d and continuing with the above example, it is assumed that the computing apparatus generated, and placed into the second column, Fourier coefficients b₀-b₃₁, which respectively correspond to the complete basis vectors \vec{V₀}-\vec{V₃₁} as described above—note that one may drop the “′” superscript after the computing apparatus moves a Fourier component b into the second column.

Therefore, the computing apparatus generates the periodic seasonal component \vec{S(t)} of [symbol] by taking the IDFT of the Fourier coefficients b₀-b₁₂ from the second column, where b₀-b₁₂ correspond to the basis functions V₀-V₁₂ and the completed basis vectors \vec{V₀}-\vec{V₁₂}. \vec{S(t)} has the same number a locations as [symbol] , but all of the locations of \vec{S(t)} are non-empty even if the non-pathological signal [symbol] has one or more empty locations.

Similarly, the computing apparatus generates the periodic first noise component [symbol] by taking the IDFT of the Fourier coefficients b₀ and b₁₃-b₂₇ from the second column, where b₁₃ b₂₇ correspond to the basis functions V₁₃-V₂₇ and the completed basis vectors \vec{V₁₃}-\vec{V₂₇}. [symbol] has the same number of locations as [symbol] , but all of the locations of [symbol] are non-empty even if the non-pathological signal [symbol] has one or more empty locations.

Furthermore, the computing apparatus generates the periodic second noise component [symbol] by taking the IDFT of the Fourier coefficients b₀ and b₂₈-b₂₉ from the second column, where b₂₈-b₂₉ correspond to the basis functions V₂₈-V₂₉ and to the complete basis vectors \vec{V₂₈}-\vec{V₂₉}, and generates the periodic third noise component [symbol] by taking the IDFT of the Fourier coefficients b₀ and b₃₀-b₃₁ from the second column, where b₃₀-b₃₁ correspond to the basis functions V₃₀-V₃₁ and to the complete basis vectors \vec{V₃₀}-\vec{V₃₁}. Both [symbol] and [symbol] have the same number of locations as [symbol] , but all of the locations of [symbol] and [symbol] are non-empty even if the non-pathological signal [symbol] has one or more empty locations.

Alternatively, the computing apparatus can generate a single periodic noise component [symbol] = [symbol] + [symbol] + [symbol] by simultaneously taking the IDFT of the Fourier coefficients b₀ and b₁₃-b₃₁ from the second column. Like \vec{S(t)}, [symbol] has the same number α of vector-element locations L as does [symbol] , but all of the locations of [symbol] are non-empty even if the non-pathological signal [symbol] has one or more empty locations.

Next, at step 134, the computing apparatus identifies and removes any outliers from the non-pathological signal, determines whether the post-outlier-removed signal is still non-pathological, and, if the signal still is non-pathological, determines whether the periodic components of the signal are to be re-determined.

At substep 134a, the computing apparatus calculates a time-domain aperiodic component of the signal (e.g., [symbol] ) to be interpolated by subtracting the one or more determined periodic components from the signal, where the aperiodic component has the same empty locations as the signal. Continuing with the above example, the computing apparatus calculates a time-domain aperiodic weather component signal \vec{A(t)} according to the following equation:

$$\vec{A ( t )} = \vec{Temp ( t )} - \vec{S ( t )} - \vec{Ns ( t )} \qquad ( 49 )$$

where [symbol] = [symbol] + [symbol] + [symbol] , \vec{A(t)} has the same number α of locations L₁-L_{α} as [symbol] , and \vec{A(t)} has empty portions in the same locations L as the empty portions of [symbol] (e.g., at least in the locations L₃, L₅, and L_{α-1} per FIG. 16).

Then, at step 134b, the computing apparatus determines the standard deviation \vec{W} or \vec{A(t)} on an element-by-element basis, and, possibly on a modulo basis if \vec{A(t)} includes more than one fundamental period (e.g., more than one year) of data.

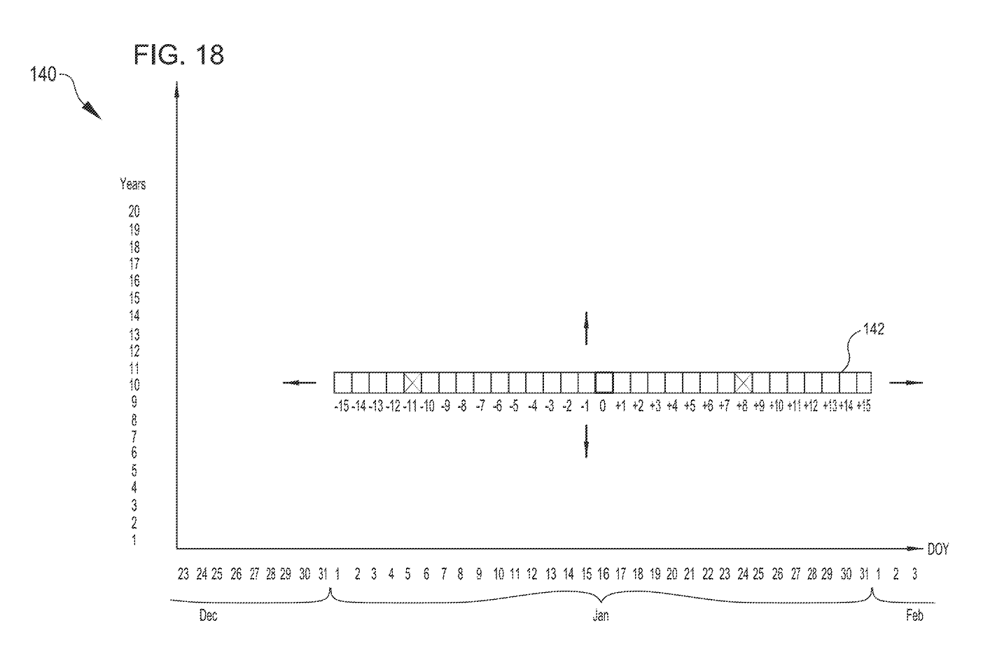

An example of this standard-deviation calculation at substep 134b is described with reference to FIG. 18, which is a plot 140 of days of the year (DOY) vs. years of data, and which includes a conceptual “window” 142 that the computing apparatus can conceptually move within the plot—here, “conceptual” indicates that in actuality, the computing apparatus may not generate a physical/displayable plot 140 or window 142, or physically move the window, but may instead generate a representation of the plot and the moveable window, or may otherwise operate in a manner that is consistent with the use of the plot and moveable window.

In this example, [symbol] and \vec{A(t)} each include α=7300 locations L₁-L_{α}, one location for each day of the year (excluding leap days) over a period of twenty years, the window 142 includes thirty one slots that are respectively labeled from −15 to +15 (including a 0^{th} slot), and the details of the calculation of the element W(DOY=January 16) of the standard deviation \vec{W} are described. Furthermore, the “X” markings indicate that \vec{A(t)}, and thus [symbol] , include empty elements for 5 January and 24 January of year 10. Alternatively, [symbol] ) and \vec{A(t)} may include a different number of locations L, and the window 142 may include a different number of slots, according to the application. And the calculations of the other elements W(DOY) of \vec{W} may be similar to the described calculation of W(DOY=January 16).

Referring to FIGS. 17 and 18, still at substep 134b and according to this example, the computing apparatus calculates each element W(DOY) of \vec{W} according to the following equation:

$$W ( DOY ) = \frac{{{\sum }_{d , y} {w}_{d} {A ( {DOY}_{y} - d )}^{2}}^{ } }{{\sum }_{d , y} {w}_{d}} \qquad ( 50 )$$

where d=−15, . . . , 0, . . . , 15 (the slots of the window 142), DOY corresponds to the 0^{th} (middle) slot of the window, y is the year index, empty elements A(DOY_{y}-d) of \vec{A(t)} and the corresponding value of d are omitted from the numerator and denominator of equation (50), and w_{d} is a weighting factor that is given by the following equation:

$${w}_{d} = 1 - {\vert \frac{d}{16} \vert}^{3} \qquad ( 51 )$$

In FIG. 18, the window 142 is shown in a position for calculating the summation Σ_{d,y}w_{d}A(DOY_{y}−d)² in the numerator of equation (50) for year y=10, where this summation is given by the following equation:

$${\sum }_{d = - 15 , y = 10}^{d = + 15} {w}_{d} {A ( 16 {Jan}_{10} - d )}^{2} = {w}_{- 15} {A ( 31 Jan )}^{2} + {w}_{- 14} {A ( 30 Jan )}^{2} + {w}_{- 13} {A ( 29 Jan )}^{2} + … + {w}_{- 9} {A ( 25 Jan )}^{2} + {w}_{- 7} {A ( 23 Jan )}^{2} + … + {w}_{0} {A ( 16 Jan )}^{2} + … + {w}_{10} {A ( 06 Jan )}^{2} + {w}_{12} {A ( 04 Jan )}^{2} + … + {w}_{15} {A ( 01 Jan )}^{2} \qquad ( 52 )$$

Note that the terms for d=−11 (5 January in year y=10) and for d=+8 (24 January in year y=10) are missing from equation (52) because these terms correspond to empty elements of \vec{A(t)}.

Furthermore, calculating the summation Σ_{d,y}w_{d} in the denominator of equation (50) for year y=10 is given by the following equation:

$${\sum }_{d = - 15 , y = 10}^{d = + 15} {w}_{d} = {w}_{- 15} + {w}_{- 14} + {w}_{- 13} + … + {w}_{- 9} + {w}_{- 7} + … + {w}_{0} + … + {w}_{10} + {w}_{12} + … + {w}_{15} \qquad ( 53 )$$

Note that the terms for d=−11 (5 January in year y=10) and for d=+8 (24 January in year y=10) are missing from equation (53) because these terms correspond to empty elements of \vec{A(t)}.

Still referring to FIG. 18 and still at substep 134b of FIG. 17, the computing apparatus continues calculating W(DOY=16 January) by effectively moving the window 142 up and down to calculate the summation components of equation (50) for the remaining years y=1, 2, . . . , 9, 11, 12, . . . , 20.

The computing apparatus calculates the elements W(DOY) of the standard deviation \vec{W} for the remaining 364 days of the year in a similar manner, by effectively moving the window 142 left and right, and up and down, within the plot 140 of FIG. 18.

And the computing apparatus calculates W(DOY=29 February) for leap days according to the following equation:

$$W ( DOY = 29 February ) = \frac{W ( DOY = 28 February ) + W ( DOY = 01 March )}{2} \qquad ( 54 )$$

Next, at substep 134c of FIG. 17, the computing apparatus determines whether \vec{A(t)} includes any outliers, and if so, it effectively removes these outliers and recalculates the periodic components of Temp\vec{(t)} if necessary.

Still at substep 134c, the computing apparatus first calculates the normalized zero-mean, unit-standard-deviation aperiodic signal [symbol] according to the following equation:

$$== \frac{\vec{A ( t )}}{\sqrt{W ( DOY )}} \qquad ( 55 )$$

where this division is done on a per-element basis, except for elements of \vec{A(t)} that are empty. For example, for the element of \vec{A(t)} that corresponds to 1 January of year y=10 (FIG. 18) of the [symbol] data, the value of this element (assuming the element is not empty) is divided by \sqrt{W(DOY=1 January)} to obtain the value of the element of [symbol] that corresponds to 1 January of year y=10.

Next, the computing apparatus identifies any outliers that are in [symbol] . For example, the computing apparatus may identify as an outlier any non-empty element [symbol] of [symbol] that satisfies the following equation:

| [symbol] |>Thresholdₒᵤₜₗᵢₑᵣ (56)

where Thresholdₒᵤₜₗᵢₑᵣ may equal any suitable number such as four.

Then, the computing apparatus converts any outlier of [symbol] and the corresponding element of the signal to be interpolated (e.g., [symbol] ), into an empty element. A reason for this is that, in an embodiment, an outlier is considered to be unreliable/invalid, as is the corresponding element of the signal to be interpolated.

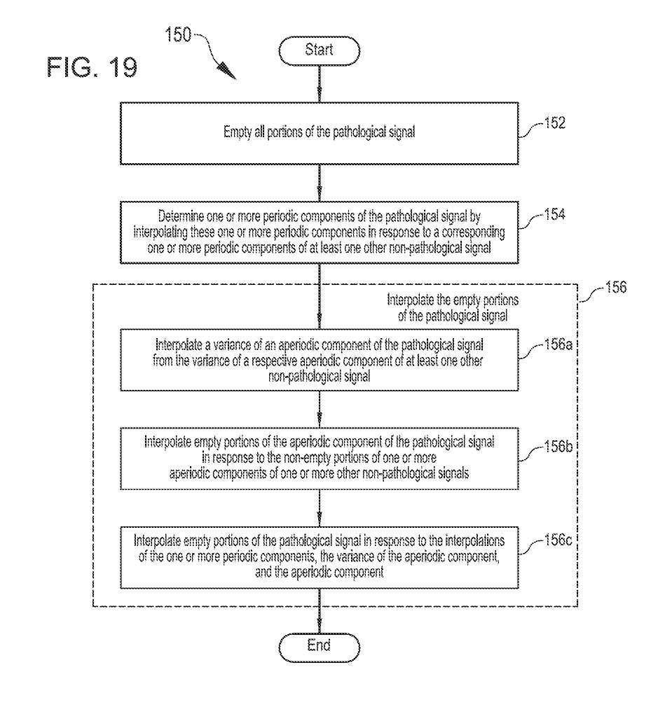

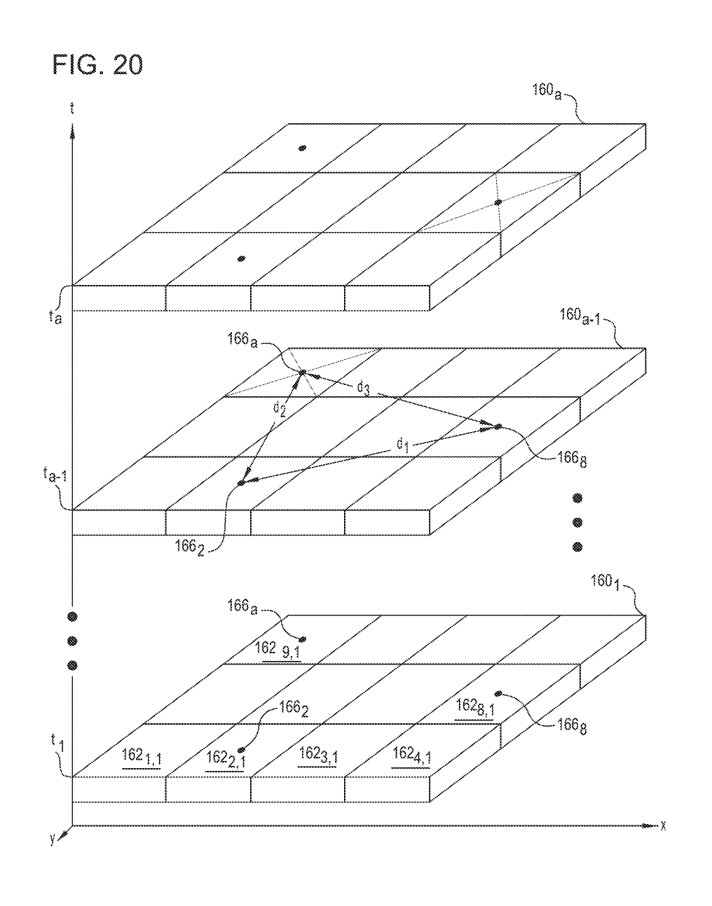

Next, at substep 134d, the computing apparatus determines whether the signal to be interpolated (e.g., [symbol] ) is still a non-pathological signal. The computing apparatus may make this determination using the algorithm discussed above in conjunction with the flow diagram 120 of FIG. 15. If the signal is still a non-pathological signal, then the computing apparatus proceeds to substep 134e. But if the signal is now a pathological signal, then the computing apparatus processes the signal (either at this time, or at some time in the future) as a pathological signal at step 136. An embodiment of a technique for processing a pathological signal is discussed below in conjunction with FIG. 19.

At substep 134e, the computing apparatus determines whether the number of identified outliers in the aperiodic component [symbol] is sufficiently large for the computing apparatus to repeat steps 132-134 of FIG. 17. The computing apparatus compares the number of outliers that it identified in substep 134c to an arbitrarily selected threshold, e.g., one. For example, if the number of outliers is greater than the threshold, then the computing apparatus repeats steps 132-134. But if the number of outliers is less than or equal to the threshold, then the computing apparatus proceeds to step 138.

At step 138, the computing apparatus interpolates the empty portions of the non-pathological signal (e.g., [symbol] ).

At substep 138a, the computing apparatus partially interpolates the empty portions of the normalized zero-mean, unit-standard-deviation aperiodic component (e.g., [symbol] in response to the non-empty portions of this aperiodic component. For example, because [symbol] is a function of time, the computing apparatus temporally interpolates the empty elements of [symbol] in response to the non-empty elements of [symbol] . The computing apparatus may use a temporal interpolation technique, such as simple kriging, that yields respective guess values Gₜ(t) and uncertainty values αₜ(t) for the empty elements of [symbol] , and that entails the computing apparatus performing the following sub-substeps of the substep 138a, which sub-substeps are omitted from the flow diagram 130 of FIG. 17:
1. Attempt to measure an auto-correlation function from [symbol] in a conventional manner.
2. If the computing apparatus is unable to measure an auto-correlation function from [symbol] , then it sets Gₜ(t)=0 (minimum certainty) and αₜ(t)=1 (maximum uncertainty) for all empty locations L of [symbol] .
3. But if the computing apparatus is able to measure an auto-correlation function from [symbol] , then it fits this auto-correlation function to a power law according to the following equation:

c(Δt)=c(Δt=1)Δt^{-α} (57)
where only α>0 are accepted, and where the maximum Δt considered is the minimum of sixty days and the first day at which the auto-correlation function becomes negative.
4. Using the fitted power law, the computing apparatus uses kriging to determine, for each empty element [symbol] of [symbol] , the guess term Gₜ(t) and the uncertainty term αₜ(t), which, in this case, is the remaining random variance.

Next, at substep 138b, the computing apparatus partially interpolates the empty portions of the normalized zero-mean, unit-standard-deviation aperiodic component (e.g., [symbol] ) in response to the non-empty portions of one or more “adjacent” normalized zero-mean, unit-standard-deviation aperiodic components. That is, the computing apparatus partially interpolates the empty portions of a signal component in response to the non-empty portions of one or more components of other signals.

For example, referring to FIG. 14, where [symbol] corresponds to the snapshots 18_{6,1}-18_{6,a} of the surface region 18₆ (e.g., FIG. 1), the computing apparatus spatially interpolates the empty elements of [symbol] in response to the corresponding non-empty elements (i.e., elements that were non-empty elements before the interpolation substep 138a) of one or more aperiodic components [symbol] corresponding to surface regions 18 that are adjacent to, or that are otherwise near, the surface region 18₆.

For example, the computing apparatus may use a spatial interpolation technique, such as simple averaging, that yields a respective value Gₜ(t) for each of the empty elements of [symbol] . Referring to FIG. 16, assume, for example purposes, that the computing apparatus is generating a value Gs_{6,1}(i) for the empty element of [symbol] that corresponds to the snap shot 18_{6,1} of the surface region 18₆ (e.g., FIG. 1). The computing apparatus generates Gs_{6,1}(t) according to the following equation:

$${Gs}_{6 , 1} = \frac{\begin{matrix}{1 , 1}_{} + {2 , 1}_{} + {3 , 1}_{} + \\ {5 , 1}_{} + {9 , 1}_{} + {11 , 1}_{} + {12 , 1}_{}\end{matrix}}{7} \qquad ( 58 )$$

where [symbol] _{1,1} is the element of the component [symbol] that corresponds to the snapshot 18_{1,1} of the surface region 18₁, [symbol] _{2,1} is the element of the component [symbol] that corresponds to the snapshot 18_{2,1} of the surface region 18₂, and so on. The computing apparatus does not use [symbol] _{7,1} in equation (58) because, as discussed above, the signal [symbol] that corresponds to the surface region 18₇ is a pathological signal; so the computing apparatus calculates no value of [symbol] _{7,1} per the equations (49) and (55) above as discussed below in conjunction with FIG. 19. Furthermore, the computing apparatus does not use [symbol] _{10,1} in equation (58) because the element of [symbol] corresponding to the snapshot 18_{10,1} of the surface region 18₁₀, and, therefore, [symbol] _{10,1}, are empty as indicated by the “X”. Moreover, if no value of [symbol] is available for any of the eight surface-region snapshots immediately surrounding the snap shot 18_{6,1}, then the computing apparatus can use elements [symbol] that correspond to not-immediately surrounding snap shots, that, e.g., form squares around the snap shot 18_{6,1}; an example of such a square is the square formed by the sixteen snap shots including the snapshots 18_{4,1}, 18_{8,1}, and 18_{12,1} (the other 13 snap shots of the square are not shown in FIG. 14).

Next, at substep 138c, the computing apparatus generates a respective interpolated value for all empty portions of the non-pathological signal to be interpolated. For example, the computing apparatus generates an interpolated temperature Tp_{interpolated}_{_{—}}_{L} for each empty location L of the non-pathological signal [symbol] according to the following equation:

Tp_{interpolated}_{_{—}}_{L}=S(L)+W(DOY_{L})·[Gₜ(L)+αₜ(L)·Gₛ(L)]+K (59)

where S(L) is the element in location L of the periodic seasonal component \vec{S(t)} (determined at step 132 above), DOY_{L} is the day of the year corresponding to the location L, W(DOY_{L}) is the standard deviation for DOY_{L} (per equation (50) above), Gₜ(L) is the value of the temporal-interpolation-guess term Gₜ(t) (determined at substep 138a above) corresponding to the location L, αₜ(L) is the value of the temporal-interpolation uncertainty αₜ(t) (determined at substep 138a above) corresponding to the location L, and Gₛ(L) is the value of the spatial-interpolation term Gₛ(t) (determined at substep 138b and equation (58) above) corresponding to the location L. And K is an optional “fudge factor”. For example, if the reason that the location L of [symbol] is empty is that cloud cover prevented the satellite 20 (e.g., FIG. 1) from measuring a surface temperature, then the actual temperature may be lower than would otherwise be interpolated with K=0, because the interpolation might be based on sunny-day temperatures. In this case, K could be a negative number to account for generally cooler land temperatures on a cloudy day.

Furthermore, in addition to interpolating the empty portions of a non-pathological signal, such as the temperature signal [symbol] , the computing apparatus may filter noise from the non-empty portions of this signal by subtracting the noise component \vec{N(t)} from the non-empty portions of this signal—the noise is already filtered from the interpolated (formerly empty) portions of the signal per equations (49) and (59).

Moreover, still referring to FIG. 17, the algorithm represented by the flow diagram 130 may be applied to a signal that has no empty portions. A reason for so applying the algorithm 130 is to determine a noise component of the signal, and then to filter the noise from the signal by subtracting the noise component from the signal. Another reason is to identify and remove any outliers from the signal; after removal of one or more outliers, the signal becomes a non-pathological signal with empty portions that the computing apparatus can interpolate per the algorithm of FIG. 17. And yet another reason is to generate an aperiodic signal component that the computing apparatus can use for interpolating empty portions of other signals. For example, the computing apparatus can generate an aperiodic component [symbol] for a non-pathological signal [symbol] corresponding to a surface region 18 (e.g., the surface region 18₁ of FIG. 14) having no empty snapshots, and can use this aperiodic component to interpolate empty portions of a signal [symbol] that corresponds to another surface region 18 (e.g., surface region 18₆ or 18₇ of FIG. 14) and that has at least one empty portion.

FIG. 19 is a flow diagram 150 of an algorithm for interpolating the empty portions of a pathological signal, such as a pathological signal [symbol] corresponding to the surface-region snapshots 18_{7,1}-18_{7,a} of FIG. 14, according to an embodiment—as discussed above in conjunction with FIG. 17, in an embodiment, a pathological signal is a signal having a number and distribution of samples insufficient to allow determination of at least one component of the signal from the signal for a particular application. In an embodiment, the computing apparatus interpolates the empty portions of the pathological signals only after it has finished interpolating the empty portions of all of the non-pathological signals. A reason for this is that the computing apparatus may use quantities generated during the interpolation of the non-pathological signals for the interpolation of the pathological signals. Alternatively, the computing apparatus may first interpolate at least the empty portions of the non-pathological signals [symbol] that respectively correspond to surface regions 18 that are adjacent to, or that are otherwise near to, the surface region 18 to which the pathological signal [symbol] corresponds.

At a step 152 of the flow diagram 150 of FIG. 19, a computing apparatus (not shown in FIG. 19) first “empties” all elements of the pathological signal. A reason for this is that because a pathological signal may include too few non-empty elements for the computing apparatus to identify and remove outliers from the signal (e.g., using the technique described above in conjunction with step 134 of FIG. 17), all elements of the signal are assumed to be unreliable.

Next, at step 154, the computing apparatus determines one or more periodic components of the pathological signal by interpolating these one or more periodic components in response to a corresponding one or more periodic components of at least one other, non-pathological signal. For example, the computing apparatus may interpolate a periodic seasonal component \vec{S(t)} for a pathological signal [symbol] that corresponds to the surface region 18₇ of FIG. 1. Referring to FIG. 14, the computing apparatus may determine \vec{S(t)} on an element-by-element, and, therefore, on a snapshot-by-snapshot, basis from the elements of the components \vec{S(t)} that correspond to surface regions 18 that are adjacent to, or that are otherwise near, the surface region 18₇. For example, the computing apparatus my spatially interpolate the \vec{S(t)} element that corresponds to the snap shot 18_{7,1} of the surface region 18₇ from the \vec{S(t)} elements that respectively correspond to the snap shots 18_{2,1}, 18_{3,1}, 18_{4,1}, 18_{6,1}, 18_{8,1}, 18_{10,1}, 18_{11,1}, and 18_{12,1} of the surrounding surface regions 18₂, 18₃, 18₄, 18₆, 18₈, 18₁₀, 18₁₁, and 18₁₂. Recall from the algorithm described above in conjunction with FIG. 17 that the periodic seasonal component \vec{S(t)} of a non-pathological signal has no empty portions. Therefore, even though the snapshot 18_{6,1} of the surface region 18₆ is marked with an “X” to indicate that the corresponding element of the non-pathological signal [symbol] is empty, the element of \vec{S(t)} corresponding to the snapshot 18_{6,1} is not empty. The computing apparatus may use any suitable interpolation technique, for example, a spatial-interpolation technique such as linear interpolation, averaging, or kriging, to interpolate the one or more periodic components of the pathological signal.

Then, at step 156, the computing apparatus interpolates the empty portions of the pathological signal

First, at substep 156a, the computing apparatus interpolates a variance of an aperiodic component of the pathological signal in response to the variance of the aperiodic component of at least one other, non-pathological signal. For example, the computing apparatus may interpolate a variance [symbol] of an aperiodic component \vec{A(t)} of a pathological signal [symbol] that corresponds a to surface region 18₇ of FIG. 1. Referring to FIG. 14, the computing apparatus may determine [symbol] on an element-by-element basis, and, therefore, on a snapshot-by-snapshot basis, from the elements of the variances [symbol] that correspond to surface regions 18 that are adjacent to, or that are otherwise near, the region 18₇. For example, the computing apparatus my spatially interpolate the [symbol] _{7,1} element that corresponds to the snapshot 18_{7,1} of the surface region 18₇ from the [symbol] elements that respectively correspond to the snapshots 18_{2,1}, 18_{3,1}, 18_{4,1}, 18_{6,1}, 18_{8,1}, 18_{10,1}, 18_{11,1}, and 18_{12,1} of the surrounding surface regions 18₂, 18₃, 18₄, 18₆, 18₈, 18₁₀, 18₁₁, and 18₁₂, respectively. Recall from the algorithm described above in conjunction with FIG. 17 that [symbol] of a non-pathological signal has no empty portions. Therefore, even though the snapshot 18_{6,1} of the surface region 18₆ is marked with an “X” to indicate that the corresponding element of the non-pathological signal [symbol] is empty, the element of [symbol] corresponding to the snapshot 18_{6,1} is not empty. The computing apparatus may use any suitable spatial-interpolation technique, such as linear interpolation, averaging, or kriging to interpolate the variance of the aperiodic component of the pathological signal. For example, the computing apparatus may compute [symbol] _{7,1} according to the following equation:

$$\vec{{W ( DOY )}_{7 , 1}} = \frac{\vec{{W ( DOY )}_{2 , 1}} + \vec{{W ( DOY )}_{3 , 1}} + \vec{{W ( DOY )}_{4 , 1}} + \vec{{W ( DOY )}_{6 , 1}} + \vec{{W ( DOY )}_{8 , 1}} + \vec{{W ( DOY )}_{10 , 1}} + \vec{{W ( DOY )}_{11 , 1}} + \vec{{W ( DOY )}_{12 , 1}}}{8} \qquad ( 60 )$$

Next, at substep 156b, the computing apparatus interpolates the empty elements of the normalized zero-mean, unit-standard-deviation aperiodic component (e.g., [symbol] of the pathological signal in response to the non-empty elements of one or more “adjacent” normalized zero-mean, unit-standard-deviation aperiodic components in a manner similar to that described above in conjunction with substep 138b of FIG. 17. That is, the computing apparatus interpolates the empty portions of a signal component (here an aperiodic component) in response to the non-empty portions of the same signal component of one or more other signals. For example, referring to FIG. 14, where [symbol] corresponds to the snapshots 18_{7,1}-18_{7,a} of the surface region 18₇ (e.g., FIG. 1), the computing apparatus spatially interpolates the empty elements of [symbol] (per above, because the signal [symbol] corresponding to the region 18₇ is pathological, all the elements of \vec{A(t)} are empty) in response to the corresponding non-empty elements (i.e., elements that were non-empty elements before the interpolation substep 138a) of one or more signals [symbol] corresponding to surface regions 18 that are adjacent to, or otherwise near to, the surface region 18₇.

For example, the computing apparatus may use a spatial interpolation technique, such as simple averaging, that yields a respective value Gₛ(t) for each of the empty elements of [symbol] . Referring to FIG. 14, assume, for example purposes, that the computing apparatus is generating a value G_{s7,1}(t) for the empty element of [symbol] that corresponds to the snapshot 18_{7,1} of the surface region 18₇ (e.g., FIG. 1). The computing apparatus generates G_{s7,1}(t) according to the following equation:

$${G}_{s7 , 1} = \frac{\begin{matrix}{2 , 1}_{} + {5 , 1}_{} + {4 , 1}_{} + \\ {8 , 1}_{} + {11 , 1}_{} + {12 , 1}_{}\end{matrix}}{6} \qquad ( 61 )$$

where [symbol] _{2,1} is the element of the component [symbol] that corresponds to the snapshot 18_{2,1} of the surface region 18₂, [symbol] _{3,1} is the element of the component [symbol] that corresponds to the snapshot 18_{3,1} of the surface region 18₃, and so on. The computing apparatus does not use [symbol] _{6,1} or [symbol] _{10,1} in equation (61) because, as discussed above, the elements of the signals [symbol] respectively corresponding to the snapshots 18_{6,1} and 18_{10,1} of the surface regions 18₆ and 18₁₀, and thus [symbol] _{6,1} and [symbol] _{10,1}, are empty as indicated by “X”. Moreover, if no value of [symbol] is available for any of the eight surface-region snapshots immediately surrounding the snap shot 18_{7,1}, then the computing apparatus can use elements of [symbol] that correspond to not-immediately surrounding snapshots, that, e.g., form squares around the snap shot 18_{7,1} as discussed above in conjunction with substep 138b of FIG. 17.

Then, at substep 156c, the computing apparatus generates a respective interpolated value for all empty elements of the pathological signal to be interpolated in a mariner similar to that described above in conjunction with substep 138c of FIG. 17.

Still at substep 156c, first, the computing apparatus effectively removes Gₜ(t) and αₜ(t) from equation (59) above by setting Gₜ(t)=0 and αₜ(t)=1 for all empty portions of the pathological signal, because the signal included (before emptying all portions of the signal per step 152 above) an insufficient number of non-empty portions to allow determining an aperiodic component of the signal from the signal itself. For example, a pathological signal [symbol] includes an insufficient number of non-empty portions to determine at least one periodic component, such as a periodic seasonal component \vec{S(t)}, of [symbol] , and thus includes an insufficient number of non-empty portions to determine an aperiodic component \vec{A(t)} of [symbol] per equation (49) above. Consequently, because the computing apparatus would generate Gₜ(t) for empty portions of \vec{A(t)} by partially interpolating from non-empty portions of \vec{A(t)}, and because there are no non-empty portions of \vec{A(t)} due to [symbol] being pathological, the temporal guess Gₜ(t) equals a minimum value of zero, and the uncertainty αₜ(t) in the temporal guess Gₜ(t) equals a maximum uncertainty value of one.

Next, still at substep 156c, the computing apparatus generates an interpolated value for each empty portion of the pathological signal. For example, the computing apparatus generates a temperature Tp_{interpolated}_{_{—}}_{L} for each empty location L of the pathological signal [symbol] according to the following equation:

Tp_{interpolated}_{_{—}}_{L}=S(L)+W(DOY_{L})·[Gₜ(L)=0+(αₜ(L)=1)·Gₛ(L)]+K (62)

which reduces to

Tp_{interpolated}_{_{—}}_{L}=S(L)+W(DOY_{L})·Gₛ(L)+K (63)

where S(L) is the element in location L of the periodic seasonal component \vec{S(t)} (interpolated at step 154 above), DOY_{L} is the day of the year corresponding to the location L, W(DOY_{L}) is the standard deviation for DOY_{L} (interpolated per substep 156a above), and Gₛ(L) is the value of the spatial-interpolation term Gₛ(t) (determined at substep 156b and equation (61) above) corresponding to the location L. And K is an optional “fudge factor” as described above in conjunction with substep 138c of FIG. 17. Furthermore, because the computing apparatus empties and then interpolates all of the elements of a pathological signal without using a noise component, the computing apparatus inherently filters noise from the pathological signal.

Still referring to FIG. 19, alternate embodiments of the algorithm represented by the flow diagram 150 are contemplated. For example, some of the described steps and substeps may be omitted, other steps and substeps may be added, and the order in which the steps and substeps are performed may be altered.

Referring to FIGS. 20-22, another application of the general interpolation techniques described above in conjunction with FIGS. 10-13 is described according to an embodiment.

FIG. 20 is a plot of snapshots 160₁-160ₐ of surface regions 162 of a landmass section 160 according to an embodiment, where the surface regions 162₂, 162₈, and 162₉ respectively include weather stations 166₂, 166₈, and 166₉, which each measure the temperature of the air above the respective surface region at each sample time t.

The air temperatures measured by each weather station 166 collectively form the elements of a respective air-temperature signal [symbol] , where “WS” indicates that the signal [symbol] corresponds to a surface region 162 that includes a weather station 166, and “X” indicates that the corresponding location L (e.g., see FIG. 16) of the signal [symbol] is empty. As discussed below, if a signal [symbol] has too few non-empty elements for determining a periodic seasonal component of [symbol] , then a computing apparatus deems this signal [symbol] to be pathological.

Furthermore, signals [symbol] correspond to surface regions 162 (FIG. 20) that include no weather stations (NWS) and, prior to interpolation, are deemed to be pathological because all of their elements are empty.

Moreover, for example purposes, it is assumed that each weather station 166 makes an air-temperature measurement once per day at the same time (i.e., the interval between successive sample times t is one day), although it is understood that the weather stations may make air-temperature measurements at different frequencies and at different times.

In addition, the areas of the surface regions 162 for air-temperature measurements may be the same as, or may be different than, the areas of the surface regions 18 (e.g., FIG. 1) for land-temperature measurements.

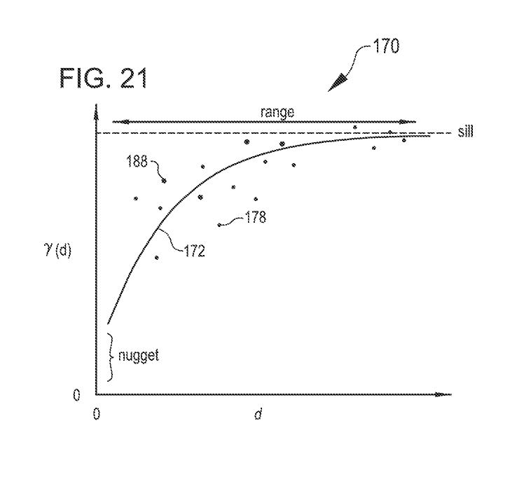

FIG. 21 is a plot 170 of a semi-variogram model 172, which a computing apparatus can generate from the air-temperature readings that the weather stations 166 of FIG. 20 make according to an embodiment.

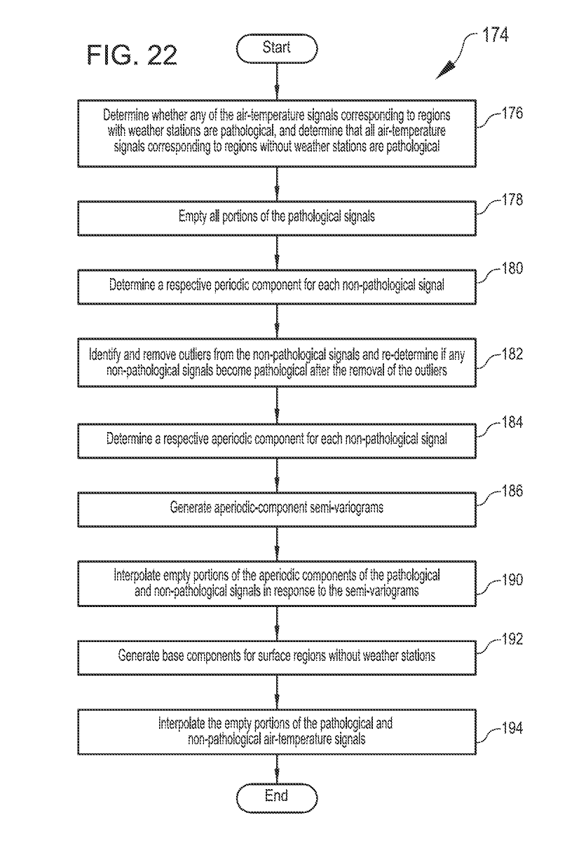

And FIG. 22 is a flow diagram 174 of an algorithm for interpolating air temperatures for the empty elements of air-temperature signals [symbol] , which, per above, correspond to the surface regions 162 (FIG. 20) that include no weather stations 166 and, therefore, are pathological, and for interpolating air temperatures for the empty elements of pathological ones of the air-temperature signals [symbol] , which, per above, correspond to surface regions 162 that include weather stations.

At step 176 of FIG. 22, a computing apparatus determines whether any of the signals [symbol] , which respectively correspond to the surface regions 162 (FIG. 20) that include weather stations 166, are pathological. For example, the computing apparatus may make this determination using an algorithm that is similar to the algorithm described above in conjunction with FIG. 15. The computing also determines that each of the signals [symbol] , which respectively correspond to the surface regions 162 that do not include weather stations 166, are pathological.

Next, at step 178, the computing apparatus empties all elements of each pathological signal [symbol] and [symbol] .

Then, at step 180, for each non-pathological signal [symbol] , the computing apparatus determines a respective periodic seasonal component [symbol] . For example, the computing apparatus may determine each periodic seasonal component [symbol] according the procedure described above at step 132 of FIG. 17.

Next, at step 182, the computing apparatus identifies and removes any outliers from each non-pathological signal [symbol] , and re-determines whether each of these signals is still non-pathological. For example, the computing apparatus may identify outliers in one or more non-pathological signals [symbol] , remove the identified outliers by emptying the corresponding elements of these one or more non-pathological signals [symbol] , and re-determine whether these one or more signals are still non-pathological, according to the procedure described above in step 134 of FIG. 17. The computing apparatus may also re-determine the periodic seasonal component of a non-pathological signal from which the computing apparatus removed one or more outliers.

Then, at step 184, the computing apparatus generates a respective aperiodic component [symbol] corresponding to each non-pathological signal [symbol] according to the following equation:

$$\vec{A\_airtempWS ( t )} = \vec{AirtempWS ( t )} - \vec{S\_airtemp ( t )} \qquad ( 64 )$$

where elements of [symbol] that correspond to empty elements of [symbol] are also empty, and where the elements of [symbol] may be referred to as “residuals”. Furthermore, in an embodiment, the periodic noise added to the air-temperature measurements by the weather stations 166 (FIG. 20) is negligible; therefore, equation (64) includes no periodic noise component in such an embodiment. Moreover, step 184 may be omitted if the computing apparatus has already generated the aperiodic components [symbol] at step 182 as part of the outlier identify-and-remove procedure.

Next, at step 186, the computing apparatus determines one or more conceptual semi-variograms 172 (FIG. 21), which represent spatial correlations of the differences between the corresponding elements (residuals) of the aperiodic components [symbol] to the distances between the corresponding weather stations 166—here, “conceptual” indicates that in actuality, the computing apparatus may not generate a physical/displayable semi-variogram, but may instead generate a representation of the semi-variogram(s), or may otherwise operate in a manner that is consistent with the use of a physical/displayable semi-variogram.

In an embodiment of a technique for interpolating air-temperature measurements, a semi-variogram is a plot of half the mean squared difference between air-temperature residuals of [symbol] over a land area as a function of distance, and provides information on the average correlation between the residuals for a group of weather stations 166 in the land area as a function of distance. As discussed below, a computing apparatus can use a semi-variogram and the residuals of the aperiodic components [symbol] for surface regions 162 (FIG. 20) that include weather stations 166 to interpolate air-temperature residuals for surface regions 162 that do not include weather stations.

Consider the following. The correlation between the temperature at a point A and the temperature at a point B may be a function of 1/d², where d is the straight-line distance between points A and B. But at some distance d, this correlation falls to zero, i.e., the temperature at point A is no indication of the temperature at point B, and vice-versa. For example, consider that the air temperature in Seattle, Wash. is no indication of the air temperature in Anchorage, Ak., because Anchorage is approximately 1400 miles from Seattle. But consider that the air temperature in Seattle is an indication of the air temperature in Redmond, Wash., because Redmond is only approximately 11 miles from Seattle. And although the air temperature in Portland, Oreg. may also be an indication of the air temperature in Redmond, which is approximately 185 miles from Portland, one would expect that the correlation of the air temperature in Seattle to the air temperature in Redmond is higher than the correlation of the air temperature in Portland to the air temperature in Redmond because Seattle is closer to Redmond than Portland is. In general, one would expect a similar correlation trend to be true for air-temperature residuals in Redmond relative to air-temperature residuals in Seattle and in Portland.

Referring to FIGS. 20 and 21, to generate an air-temperature-residual semi-variogram for a land-section snapshot 160₁, for example, the computing apparatus first generates a respective point \hat{γ}(d) 188 on the plot 170 for each pair of the weather stations 166₂, 166₈, and 166₉ according to the following equation:

\hat{γ}(d)=|A_airtempWS(t₁)_{WS1}−A_airtempWS(t₁)_{WS2}|² (65)

where d is the distance between weather stations WS1 and WS2, A_airtempWS(t)_{WS1} is the air-temperature residual of [symbol] _{WS1} corresponding to the snapshot 160₁, A_airtempWS(t)_{WS2} is the air-temperature residual of [symbol] _{WS2} corresponding to the snapshot 160₁, and the following three pairs of weather stations 166 are respectively represented by WS1 and WS2: 166₂ and 166₈ (d=d₁), 166₂ and 166₉ (d=d₂), and 166₈ and 166₉ (d=d₃). Therefore, in this example, the computing apparatus generates three points 188 on the plot 170 for the snapshot 160₁: points \hat{γ}(d₁), \hat{γ}(d₂), and \hat{γ}(d₃). But in another example, the snapshots 160 may include more or fewer than twelve surface regions 162 and more or fewer than three weather stations 166, and, therefore, the computing apparatus may generate more or fewer than three points on the plot 170 of FIG. 21.

Alternatively, to generate the air-temperature-residual semi-variogram for the land-section snapshot 160₁, the computing apparatus may generate a respective point 188 on the plot 170 for each pair of the weather stations 166₂, 166₈, and 166₉ in multiple snapshots according to the following equation:

$$\hat{γ} ( d ) = \frac{\begin{matrix}\begin{matrix}{\vert A\_airtempWS \text{?} - A\_airtempWS \text{?} \vert}^{2} + \\ {\vert A\_airtempWS \text{?} - A\_airtempWS \text{?} \vert}^{2} + … +\end{matrix} \\ {\vert A\_airtempWS ( {t}_{n} ) \text{?} - A\_airtempWS ( {t}_{n} ) \text{?} \vert}^{2}\end{matrix}}{n} \\ \text{?} \text{indicates text missing or illegible when filed} \qquad ( 66 )$$

where n is the number of snapshots over which residuals A_airtempWS(t)_{WS1} and [symbol] _{WS2} are used. But as in the previous example, the computing apparatus generates three conceptual points 188 on the plot 170 corresponding to the three pairs of the weather stations 166₂, 166₈, and 166₉.

In yet another alternative, the computing apparatus may generate a respective air-temperature-residual semi-variogram for each day of the year. For example, to generate an air-temperature-residual semi-variogram for 1 October, the computing apparatus may generate a respective point 188 on the plot 170 for each pair of the weather stations 166₂, 166₈, and 166₉ in multiple snapshots that each correspond to 1 October in different years according to the following equation:

$$\hat{γ} ( d ) = \frac{\begin{matrix}{\vert {A}_{{airtempWS ( 01 October Year 1 )}_{WS 1}} - {A}_{{airtempWS ( 01 October Year 1 )}_{WS 2}} \vert}^{2} + \\ \vert {A}_{{airtempWS ( 01 October Year 2 )}_{WS 1}} - {A}_{{airtempWS ( 01 October Year 2 )}_{WS 2}} \vert + \\ {\vert A\_airtempWS {( 01 October Year y )}_{WS 1} - A\_airtempWS {( 01 October Year y )}_{WS 2} \vert}^{2}\end{matrix}}{y} \qquad ( 67 )$$

where y is the number of years over which residuals A_{—airtempWS(t)}_{WS1} and [symbol] _{WS2} are used. But as in the previous example, the computing apparatus generates three conceptual points 188 on the plot 170 corresponding to the three pairs of the weather stations 166₂, 166₈, and 166₉.

Next, still at step 186 of FIG. 22, and referring to FIG. 21, the computing apparatus fits a conventional semi-variogram to the plotted points 188. For example, the computing apparatus may fit to the plotted points 188 the general semi-variogram given by the following equation:

$$γ ( d ) = γ ( ∞ ) ( 1 - \frac{1}{1 - {( d / λ )}^{2}} ) \qquad ( 68 )$$

where d is distance from a weather station 166 to a particular surface region 162 (FIG. 20), γ(∞) (the sill) is the maximum distance at which a correlation exists between the air-temperature residual at the weather station and the air-temperature residual at the particular surface region, and λ is the half-correlation distance. Furthermore, referring to FIG. 21, the range is the distance d at which γ(d) approximately equals the sill (and at which there is no longer a correlation between air-temperature residuals), and the nugget is the value of γ(d) at d=0—although according to equation (67) γ(0)=0 (i.e., the nugget=0), sometimes the semi-variogram model yields a nonzero value for γ(0) (i.e., nugget>0).

Then, at step 190 of FIG. 22, the computing apparatus interpolates a respective air-temperature aperiodic component [symbol] for each surface region 162 that does not include a weather station 166, interpolates a respective air-temperature aperiodic component [symbol] for each surface region that does include a weather station but that corresponds to a pathological signal [symbol] , and interpolates the empty elements of the aperiodic components [symbol] for surface regions that do include weather stations but that correspond to non-pathological signals [symbol] . For example, to interpolate an element of [symbol] corresponding to the snapshot 162_{1,1} of the surface region 162₃, the computing apparatus may spatially krig this element using only the semi-variogram generated for the land-section snapshot 160₁. Alternatively, the computing apparatus may spatially and temporally krig this element using the semi-variogram generated for the land-section snapshot 160₁ and one or more of the semi-variograms generated for the land-section snapshots 160²-160ₐ.

After the completion of step 190, there is an aperiodic component [symbol] with no empty portions for each surface region 162 that includes a weather station 166, and an aperiodic component [symbol] for each surface region that does not include a weather station.

But at least for the surface regions 162 with no weather stations 166, there are no components (e.g., periodic seasonal components \vec{S(t)}) to which the computing apparatus call add the aperiodic components [symbol] to obtain interpolated air temperatures.

Therefore, at step 192, the computing apparatus generates a respective air-temperature base component [symbol] for each surface region 162 that has no weather station. For example, the computing apparatus may generate the elements of the base component [symbol] for a surface region 162 from the average air temperatures for that surface region over the period from t₁ to t_{α}; sources of such average-air temperatures include the Worldlier high-resolution climatology data set, which has a monthly time resolution and a thirty-arcsecond spatial resolution. If the dimensions of the surface regions 162 are not the same as the spatial resolution of the base air temperatures, or the temporal resolution (i.e., the interval between successive sample times t) of the land-section snap shots 160 is not the same as the temporal resolution of the base air temperatures, then the computing apparatus may apply to the base air temperatures any suitable interpolation technique that yields the base components [symbol] having the same spatial and temporal resolutions as the surface regions 162 and the land-section snapshots 160, respectively. The computing apparatus may also generate base components [symbol] for the surface regions 162 that include weather stations 164 in a similar manner.

Next, at step 194, the computing apparatus generates a respective interpolated air-temperature signal [symbol] for each of the surface regions 162 (FIG. 20) that include no weather station, according to the following equation:

$$\vec{AirtempNWS ( t )} = \vec{A\_airtempNWS ( t )} + \vec{B\_airtempNWS ( t )} \qquad ( 69 )$$

Then, at step 196, the computing apparatus generates a respective interpolated air-temperature signal [symbol] for each surface region 162 that includes a weather station as follows. For each non-empty element of [symbol] , the computing apparatus leaves unchanged the value of that element, which is an air temperature measured by the corresponding weather station. But for each empty element of [symbol] , the computing apparatus generates an interpolated value for that element according to the following equation:

AirtempNWS(t)=A_airtempWS(t)+S_airtemp(t) (70)

Alternatively, if at step 194 the computing apparatus determined respective base components [symbol] for the surface regions that include weather stations, then the computing apparatus may generate an interpolated value for each empty element of [symbol] according to the following equation:

AirtempWS(t)=A_airtempWS(t)+B_airtempWS(t) (71)

Referring to FIGS. 20-22, alternate embodiments are contemplated. For example, although shown as belonging to a single landmass section 160, one or more of the weather stations 166₂, 166₈, and 166₉ may belong to multiple landmass sections for computational purposes, and the computing apparatus may interpolate air temperatures for each of the surface regions 162₂, 162₈, and 162₉ by combining (e.g., averaging) the multiple interpolation results for that surface region, where each interpolation result corresponds to a respective one of the landmass sections to which the surface region belongs. Furthermore, some of the described steps may be omitted, other steps may be added, and the order in which the steps are performed may be altered.

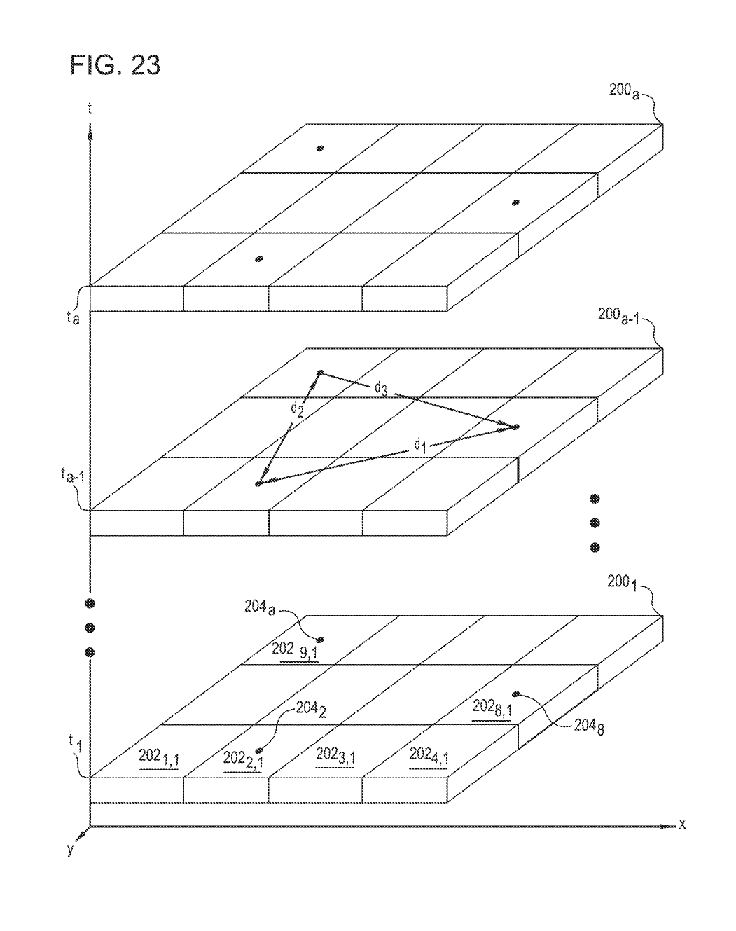

Referring to FIGS. 23-26, yet another application of one or more of the general interpolation techniques described above in conjunction with FIGS. 10-13 is described according to an embodiment.

In the below-described application, a computing apparatus may interpolate dew points that correspond to surface regions of a landmass section.

A dew point is the temperature to which a given volume of an air/water-vapor mixture must be cooled, at a constant barometric pressure, for the water vapor to condense into liquid water. That is, the dew point is the water-vapor-saturation temperature of a volume of air that contains water vapor.

FIG. 23, which is similar to FIG. 20, is a plot of snapshots 200₁-200ₐ of a landmass section 200, which includes surface regions 202 of the earth according to an embodiment, where the surface regions 202₂, 202₈, and 202₉ respectively include weather stations 204₂, 204₈, and 204₉, which each measure the dew point of the air above the respective surface region at each sample time t₁-tₐ. Each weather station 204 may measure the dew point instead of, or in addition to, the temperature of the air above the corresponding surface region.

The dew points measured by each weather station 204 collectively form the elements of a respective dew-point signal [symbol] where “WS” indicates that the signal [symbol] corresponds to a surface region 202 that includes a weather station 204, and “X” indicates that the corresponding location L (e.g., see FIG. 16) of the corresponding signal [symbol] is empty.

As discussed below, if a signal [symbol] has a characteristic, such as too few non-empty elements, that renders a computing apparatus unable to determine whether the elements of the signal are valid, then the computing apparatus deems this signal [symbol] to be pathological.

Furthermore, signals [symbol] respectively correspond to surface regions 202 that include no weather stations (“NWS” indicates no weather station), and, that prior to interpolation, are deemed to be pathological because all of their elements are empty.

Moreover, for example purposes, it is assumed that each weather station 204 makes a dew-point measurement once per day at the same time (i.e., the interval between successive sample times t is one day), although it is understood that the weather stations may make dew-point measurements at different frequencies and at different times.

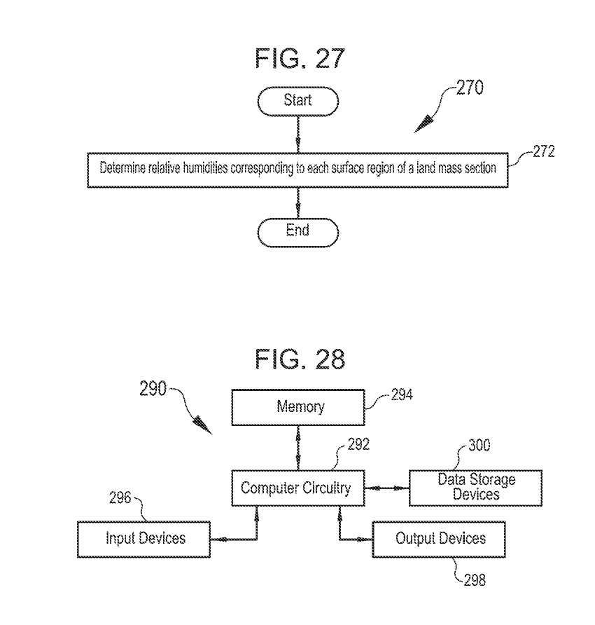

In addition, the areas of the surface regions 202 for dew-point measurements may be the same as, or different than, the areas of the surface regions 18 (e.g., FIG. 1) for land-temperature measurements, and may be the same as, or different than, the areas of the surface regions 162 (FIG. 20) for air-temperature measurements. But if the computing apparatus also determines a relative humidity of the air over a surface region 202, then one can select surface regions 162 and 202 having the same areas for air-temperature and dew-point measurements as further discussed below in conjunction with FIG. 27.

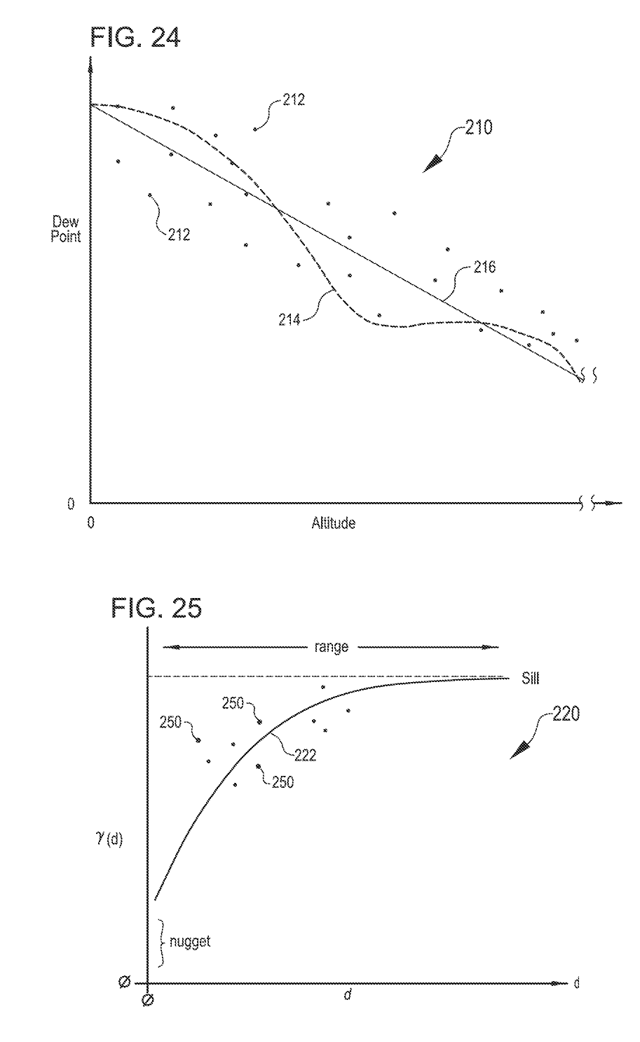

FIG. 24 is a plot 210 of dew points 212 versus altitudes of the respective weather stations 204 (FIG. 23) that measured the dew points during a particular day of the year (e.g., 25 February), a curve 214 conventionally fitted to the points, and a filtered version 216 of the fitted curve 214, according to an embodiment.

FIG. 25 is a plot 220 of a semi-variogram model 222, which a computing apparatus can conceptually generate in response to the dew-point measurements that the weather stations 204 of FIG. 23 make, according to an embodiment—here, “conceptually” indicates that in actuality, the computing apparatus may not generate a physical/displayable plot 220 of the semi-variogram model 222, but may instead generate a representation of the plot, or may otherwise operate in a manner that is consistent with the use of the plot.

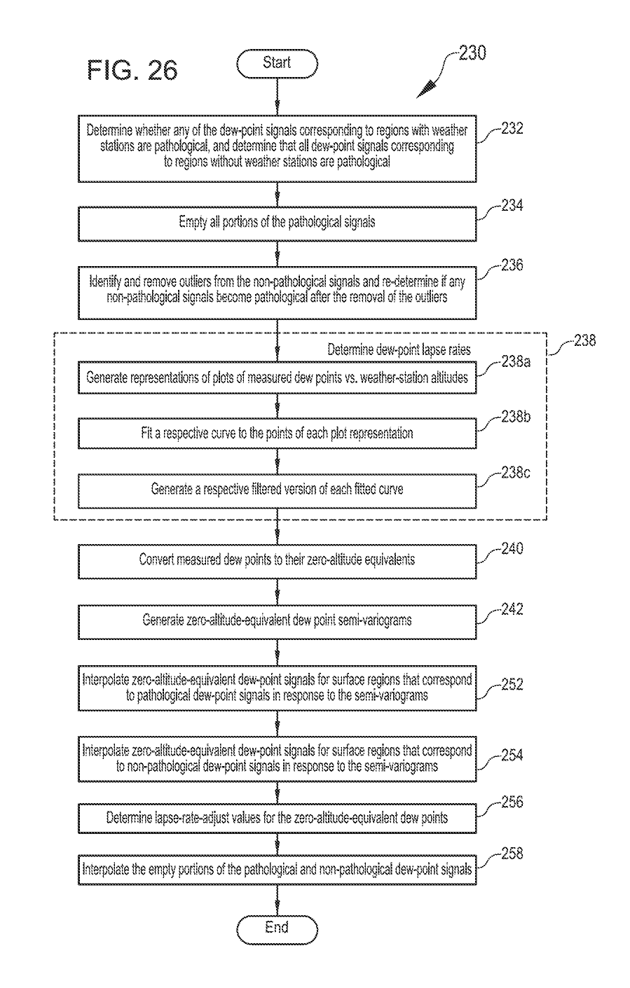

And FIG. 26 is a flow diagram 230 of an algorithm for interpolating dew points for the empty elements of dew-point signals [symbol] , which, per above, correspond to the surface regions 202 (FIG. 23) that include no weather stations, and for interpolating dew points for the empty elements of pathological and non-pathological dew-point signals [symbol] , which, per above, correspond to surface regions that include weather stations.

At step 232 of the flow diagram 230 of FIG. 26, a computing apparatus determines whether any of the signals [symbol] , which respectively correspond to the surface regions 202 (FIG. 23) that include weather stations 204, are pathological. For example, the computing apparatus may make this determination using an algorithm that is similar to the algorithm described above in conjunction with FIG. 15. The computing apparatus also determines that each of the signals [symbol] , which respectively correspond to the surface regions 202 that do not include weather stations 204, are pathological.

Next, at step 234, the computing apparatus empties all elements of each pathological signal [symbol] and [symbol] .

Then, at step 236, the computing apparatus identifies and removes any outliers from each non-pathological signal [symbol] , and re-determines whether each of these signals is still non-pathological. For example, the computing apparatus may identify outliers in one or more non-pathological signals [symbol] , remove the identified outliers by emptying the corresponding elements of these one or more non-pathological signals [symbol] , and re-determine whether these one or more signals are still non-pathological, according to the procedure described above in conjunction with step 134 of the flow chart 130 of FIG. 17.

Next, at step 238, the computing apparatus determines the respective dew-point lapse rate (the dew point versus altitude) for the landmass section 200 (FIG. 23) for each day of the year.

At substep 238a of FIG. 26, and referring to FIG. 24, for each day of the year, the computing apparatus generates, from the non-pathological signals [symbol] , the conceptual plot 210 of the points 212, where each point represents a respective non-empty element (i.e., dew-point measurement) that corresponds to the particular day of the year, versus the altitude of the respective weather station 204 that generated the element (i.e., made the dew-point measurement)—here, “conceptual” indicates that in actuality, the computing apparatus may not generate a physical/displayable plot 210 of the points 212, but may instead generate a representation of the plot, or may otherwise operate in a manner that is consistent with the use of the plot. For example, if the [symbol] time period t₁-t_{α} spans ten years, then, for any day of the year, such as 25 February, for which the signals [symbol] include no empty elements, then the number of plotted elements 212 equals ten times the number of weather stations 204 located within the boundaries of the landmass section 200; if one or more of the signals [symbol] includes an empty element for the day of the year then the number of points 212 is reduced by the number of empty elements. Furthermore, if a weather station 204 is within a particular range, such as approximately ten meters, of the ground, then the computing apparatus may equate the altitude of the weather station with the altitude of the surface region 202 that includes the weather station. Moreover, the altitudes of the weather stations may be relative to any reference, such as sea level.

Next, at substep 238b, for each plot 210 (one plot for each day of the year in this example), the computing apparatus fits a respective conceptual curve 214 to the points 212, where the curve extends at least to the origin (altitude equals zero); the curve may extend below the origin (altitude less than zero) in cases where the altitude of one or more of the surface regions 202 or weather stations 204 is below the altitude reference (e.g., sea level)—here, “conceptual” indicates that in actuality, the computing apparatus may not generate a physical/displayable curve 214, but may instead generate a representation of the curve, or may otherwise operate in a manner that is consistent with the use of the curve. Although disclosed as being continuous, the curve 214 may be discrete, having values, for example, at every foot of altitude.

Then, at substep 238c, the computing apparatus filters each fitted curve 214 to obtain a respective conceptual smoothened curve 216, which represents the dew-point lapse rate for a corresponding day of the year for the landmass section 200—here, “conceptual” indicates that in actuality, the computing apparatus may not generate a physical/displayable smoothened curve 216, but may instead generate a representation of the smoothened curve, or may otherwise operate in a manner that is consistent with the use of the smoothened curve. For example, where the fitted and smoothened curves 214 and 216 are discrete, then the computing apparatus may apply a conceptual x-day window filter (e.g., a window filter similar to the 31-day window filter 142 of FIG. 18) to each point of the fitted curve, where the window's center slot is aligned with the point. First, the computing apparatus determines the median of the values in the window and assigns the median to the center slot of the window, effectively slides the window over the fitted curve 214 so that the center slot is effectively aligned with another point of the fitted curve, and repeats this procedure until all of the points of the fitted are similarly processed; the median values form a discrete intermediate curve (not shown in FIG. 24). Next, the computing apparatus effectively places the window filter over the intermediate curve, takes the average of the values in the window, assigns the average to the center slot of the window, slides the window over the intermediate curve so that the center slot is aligned with another point of the intermediate curve, and repeats this procedure until all of the points of the intermediate curve are similarly processed; these average values form the smoothened curve 216. Although the smoothened curve 216 is disclosed as being a straight line for example purposes, the smoothened curve may have any other suitable shape.

Next, at step 240 of FIG. 26, the computing apparatus converts each non-empty dew-point element of the signals [symbol] to its zero-altitude equivalent in response to the respective filtered lapse-rate curve 216 of FIG. 24. In an embodiment, the computing apparatus may determine the zero-altitude equivalent DP₀_{_{—}}ₐₗₜ of a dew point DP according to the following equation:

DP₀_{_{—}}ₐₗₜ=DP−Alt_{weather}_{_{—}}ₛₜₐₜᵢₒₙ·CurveSlope (72)

where Alt_{weather}_{_{—}}ₛₜₐₜᵢₒₙ is the altitude of the weather station 204 (FIG. 23) that generated the dew point DP, and CurveSlope is the slope of the corresponding smoothened lapse-rate curve 216 at Alt_{weather}_{_{—}}ₛₜₐₜᵢₒₙ. For example, suppose that a weather station 204 located at an altitude of 1000 meters (m) measures a dew point of 0° C. on 25 February of year 1 of the period t₁-t_{α}, and that the smoothened lapse-rate curve 216 for 25 February is a straight line having a slope of −1.0° C./100 m. Therefore, the zero-altitude equivalent of the 0° C. dew point measured by the weather station 204 is equal to 0° C.−(1000 m(−1.0° C./100 m)=10° C. The computing apparatus generates, from the calculated zero-altitude-equivalent dew points, signals [symbol] , which respectively correspond to the surface regions 202 that include weather stations 204 (FIG. 23). Each signal [symbol] has empty portions that correspond to the empty portions of the corresponding signal [symbol] for the same surface region 202.

Then, at step 242, the computing apparatus determines of one or more conceptual semi-variograms 222 (FIG. 25), which represent spatial correlations of the differences between the corresponding elements (i.e., the corresponding zero-altitude-equivalent dew points) of the signals [symbol] to the distances between the corresponding weather stations 204.

In this example, a semi-variogram is a plot of half the mean squared difference between zero-altitude-equivalent dew points of the signals [symbol] over the landmass section 200 as a function of distance, and provides information on the average correlation between the zero-altitude-equivalent dew points for a group of weather stations 204 (e.g., the weather stations 204₂, 204₈, and 204₉ of FIG. 23) in the landmass section as a function of distance. As discussed below, a computing apparatus can use a semi-variogram and the zero-altitude-equivalent dew points of the signals [symbol] that correspond to the surface regions 202 (e.g., the surface regions 202₂, 202₈, and 202₉ of FIG. 23) that include weather stations 204 to interpolate zero-altitude-equivalent dew points for the surface regions that do not include weather stations.

Consider the following. The correlation between the dew point at a point A and the dew point at a point B may be a function of 1/d², where d is the straight-line distance between points A and B. But at some distance d, this correlation falls to zero, i.e., the dew point at point A is no indication of the dew point at point B, and vice-versa. For example, consider that one would expect that the dew point in Seattle, Wash. is no indication of the dew point in Anchorage, Ak., because Anchorage is approximately 1400 miles from Seattle. But consider that one would expect that the dew point in Seattle is an indication of the dew point in Redmond, Wash., because Redmond is only approximately 11 miles from Seattle. And although the dew point in Portland, Oreg. may also be an indication of the dew point in Redmond, which is approximately 185 miles from Portland, one would expect that the correlation of the dew point in Seattle to the dew point in Redmond is higher than the correlation of the dew point in Portland to the dew point in Redmond because Seattle is closer to Redmond than Portland is. In general, one would expect that a similar correlation trend is true for zero-altitude-equivalent dew points for Redmond relative to zero-attitude-equivalent dew points for Seattle and for Portland.

Referring to FIGS. 23 and 25, to generate a conceptual zero-altitude-equivalent dew-point semi-variogram for a landmass-section snapshot 200₁, for example, the computing apparatus first generates a respective conceptual point \hat{γ}(d) 250 on the conceptual plot 220 for each pair of the weather stations 204₂, 204₈, and 204₉ according to the following equation:

\hat{γ}(d)=|DewpointWS_zeroaltitude(t₁)_{WS1}−DewpointWS_zeroaltitude(t₁)_{WS2}|² (73)

where d is the distance between weather stations WS1 and WS2, DewpointWS_zeroaltitude(t₁)_{WS1} is the zero-altitude-equivalent dew point of [symbol] _{WS1} corresponding to the snap shot 200₁, DewpointWS_zeroaltitude(t₁)_{WS2} is the zero-altitude-equivalent dew point of [symbol] _{WS2} corresponding to the snap shot 200₁, and the following three pairs of weather stations 204 are respectively represented by WS1 and WS2: 204₂ and 204₈ (d=d₁), 204₂ and 204₉ (d=d₂), and 204₈ and 204₉ (d=d₃). Therefore, in this example, the computing apparatus generates three conceptual points 250 on the plot 220 for the snapshot 200₁, points \hat{γ}(d₁), \hat{γ}(d₂), and \hat{γ}(d₃). But in another example, the snapshots 200 can include more or fewer than twelve surface regions 202 and more or fewer than three weather stations 204, and, therefore, the computing apparatus can generate more or fewer than three points on the plot 220.

Alternatively, to generate the zero-altitude-equivalent dew-point semi-variogram for the landmass snapshot 200, the computing apparatus can generate a respective conceptual point 250 on the plot 220 for each pair of the weather stations 204₂, 204₈, and 204₉ in multiple snapshots according to the following equation:

$$\hat{γ} ( d ) = \frac{\begin{matrix}{\vert {DewpointWS}_{{zeroaltitude ( {t}_{1} )}_{WS 1}} - {DewpointWS}_{{zeroaltitude ( {t}_{1} )}_{WS 2}} \vert}^{2} + \\ \vert {DewpointWS}_{{zeroaltitude ( {t}_{2} )}_{WS 1}} - {DewpointWS}_{zero} + … + \\ {\vert DewpointWS\_zeroaltitude {( {t}_{n} )}_{WS 1} - DewpointWS\_zeroaltitude {( {t}_{2} )}_{WS 2} \vert}^{2}\end{matrix}}{( n )} \qquad ( 74 )$$

where n is the number of snapshots over which zero-altitude-equivalent dew points DewpointWS_zeroaltitude(t)_{WS1} and \vec{Dewpoint_zeroaltitude(t)}_{WS2} are used. But as in the previous example, the computing apparatus generates three points 250 on the plot 220 in response to the three weather stations 204₂, 204₈, and 204₉.

In yet another alternative, the computing apparatus may generate a respective zero-altitude-equivalent dew-point semi-variogram for each day of the year. For example, to generate a zero-altitude-equivalent dew-point semi-variogram for 1 October, the computing apparatus may generate a respective point 250 on the plot 220 for each pair of the weather stations 204₂, 204₈, and 204₉ in multiple snapshots that each correspond to 1 October in different years according to the following equation:

$$\hat{γ} ( d ) = \frac{\begin{matrix}{\vert {DewpointWS}_{{zeroaltitude ( 01 October year 1 )}_{WS 1}} - {DewpointWS}_{{zeroaltitude ( 01 October year 1 )}_{WS 2}} \vert}^{2} + \\ {\vert {DewpointWS}_{{zeroaltitude ( 01 October year 2 )}_{WS 1}} - {DewpointWS}_{{zeroaltitude ( 01 October year 2 )}_{WS 2}} \vert}^{2} + … + \\ {\vert {DewpointWS}_{\_zeroaltitude {( 01 October year y )}_{WS 1}} - {DewpointWS}_{\_zeroaltitude {( 01 October year y )}_{WS 2}} \vert}^{2}\end{matrix}}{y} \qquad ( 75 )$$

where y is the number of years over which zero-altitude-equivalent dew points DewpointWS_zeroaltitude(t)_{WS1} and DewpointWS_zeroaltitude(t)_{WS2} are used, But as in the previous example, the computing apparatus generates three points 250 on the plot 220 corresponding to the three pairs of the weather stations 204₂, 204₈, and 204₉.

Next, still at step 242 of FIG. 26, and referring to FIG. 25, the computing apparatus fits a conventional conceptual semi-variogram to the conceptual plotted points 250. For example, the computing apparatus may fit to the representations of the plotted points 250 the general semi-variogram given by the following equation:

$$γ ( d ) = γ ( ∞ ) ( 1 - \frac{1}{1 - {( d / λ )}^{2}} ) \qquad ( 76 )$$

where d is distance from a weather station 204 to a particular surface region 202 (FIG. 23), γ(∞) (the sill) is the maximum distance at which a correlation exists between the zero-altitude-equivalent dew point at the weather station and the zero-altitude-equivalent dew point at the particular surface region, and λ is the half-correlation distance. Furthermore, referring to FIG. 25, the range is the distance d at which γ(d) approximately equals the sill (and at which there is no longer a correlation between zero-altitude-equivalent dew points), and the nugget is the value of γ(d) at d=0—although according to equation (76) γ(0)=0 (i.e., nugget=0), sometimes the semi-variogram model yields a nonzero value for γ(0) (i.e., nugget>0).

Then, at step 252 of FIG. 26, the computing apparatus interpolates a respective zero-altitude-equivalent dew-point signal [symbol] for each surface region 202 that does not include a weather station 204, and interpolates a respective zero-altitude-equivalent dew-point signal [symbol] for each surface region that does include a weather station but that corresponds to a pathological signal [symbol] . For example, to interpolate an element of [symbol] corresponding to the snap shot 202_{3,1} of the surface region 202₃ (FIG. 23), the computing apparatus may spatially krig this element using only the semi-variogram generated for the landmass-section snapshot 200₁. Alternatively, the computing apparatus may spatially and temporally krig this element using the semi-variogram generated for the landmass-section snap shot 200₁ and one or more of the semi-variograms generated for the landmass-section snap shots 200₂-200ₐ.

Next, at step 254 of FIG. 26, the computing apparatus interpolates a respective zero-altitude-equivalent dew-point signal [symbol] for each surface region 202 that includes a weather station 204 and that corresponds to a non-pathological signal [symbol] . The computing apparatus interpolates a respective zero-altitude-equivalent dew point for each empty element of each signal [symbol] , for example, according to the same procedure described above in conjunction with step 252.

Alternatively, the computing apparatus my interpolate zero-altitude-equivalent dew points for the empty portions of the non-pathological signals [symbol] according to a procedure that is similar to that described above in conjunction with the algorithm of FIG. 17. First, the computing apparatus determines a periodic seasonal component for each signal [symbol] , and determines residuals for the non-empty elements of each of these signals, in a manner similar to that described above in conjunction with steps 132 and 134 of FIG. 17—the computing apparatus may have already calculated these periodic seasonal components and residuals if, in step 236 above, it identified and removed outliers from the signals [symbol] in a manner similar to that described above in conjunction with step 134 of FIG. 17. Next, for each day of the year, the computing apparatus generates a semi-variogram for the residuals in a manner similar to that described above in conjunction with step 242, spatially interpolates first partial components of the residuals for the empty portions of each signal [symbol] in response to the semi-variograms per step 252 above, temporally interpolates second partial components of the residuals for the empty portions of each signal [symbol] in response to the residuals for the non-empty portions of the same signal per step 136a of FIG. 17, and then interpolates the residuals for the empty portions of each signal [symbol] in a manner similar to that described above in conjunction with step 136c of FIG. 17.

After the completion of steps 252 and 254, there is a zero-altitude-equivalent dew-point signal [symbol] having no empty portions for each surface region 202 that includes a weather station 204, and a zero-altitude-equivalent dew-point signal [symbol] having no empty portions for each surface region 202 that does not include a weather station.

Then, at step 256, the computing apparatus computes a lapse-rate-adjust value LRA for each element of the zero-altitude-equivalent dew-point signals [symbol] , and for each element of the zero-amplitude-equivalent dew-point signals [symbol] that corresponds to an empty portion (i.e., no valid dew-point measurement) of the corresponding dew-point signal [symbol] , according to the following equation:

LRA=Alt_{surface}_{_{—}}_{region}·CurveSlope (78)

where Alt_{surface}_{_{—}}_{region} is the altitude of the corresponding surface region 202 (FIG. 23), and CurveSlope is the slope of the corresponding smoothened lapse-rate curve 216 of FIG. 24 at the altitude Alt_{surface}_{_{—}}_{region}. For example, suppose that the surface region 202₁ has an altitude of 1000 m above sea level, and that the lapse-rate curve 216 for the landmass section 200₁ on 25 February is a straight line having a slope of −1.0° C./100 m. Therefore, the lapse-rate-adjust value LRA for the surface region 202₁ is equal to 1000 m(−1.0° C./100 m)=−10° C.

Next, at step 258, the computing apparatus generates a respective interpolated dew-point signal [symbol] for each surface region 202 that does not include a weather station 204, and generates a respective interpolated dew-point signal [symbol] for each surface region 202 that includes a weather station and for which the corresponding original dew-point signal [symbol] includes at least one empty portion. For example, the computing apparatus generates the interpolated dew-point signals [symbol] and [symbol] by combining (e.g., adding) the LRA values calculated per step 256 above with the elements of the correspond signals [symbol] and [symbol] on an element-by-element basis. For example, if LRA=−10° C. for the surface region 202₁ on 25 February, and the zero-altitude-equivalent dew point for this surface region equals 15° C. on 25 February of year 05 of a ten-year period t₁-t_{α}, then the interpolated dew point for the surface region 202₁ on 25 February in year 05 equals 15° C.+(−10° C.)=5° C. And, as stated above, for a day on which a weather station 204 yielded a valid dew-point measurement, then the computing apparatus sets the interpolated dew point for that surface region on that day equal to the valid measured dew point.

Referring to FIGS. 23-26, alternate embodiments of the dew-point interpolation algorithm of FIG. 26 are contemplated. For example, although shown as belonging to a single landmass section 200, one or more of the weather stations 204₂, 204₈, and 204₉ may belong to multiple landmass sections for computational purposes, and the computing apparatus may interpolate dew points for each of the surface regions 202₂, 202₈, and 202₉ by combining (e.g., averaging) the multiple interpolation results for that surface region, where each interpolation result corresponds to a respective one of the landmass sections to which the surface region belongs. Furthermore, some of the described steps may be omitted, other steps may be added, and the order in which the steps are performed may be altered. For example, substep 238c may be omitted, and the computing apparatus may use the unfiltered lapse-rate curve 214 (FIG. 24) for determining zero-altitude-equivalent dew points.

FIG. 27 is a flow-diagram 270 of an algorithm for determining a relative-humidity signal [symbol] for each surface region 202 of FIG. 23 according to an embodiment.

Relative humidity describes the amount of water vapor in a volume of an air/water-vapor mixture, is defined as the percentage ratio of the partial pressure of water vapor in the volume to the saturated water-vapor pressure of the volume, and is a function of the temperature and the pressure of the volume. In terms of the dew point of the volume of the air/water-vapor mixture, relative humidity RH is a measure of how close the current temperature of the volume is to the dew point of the volume, and may be given by the following equation:

$$RH = {e}^{\frac{- cb ( T - {T}_{d} )}{( b + {T}_{d} ) ( b + T )}} \times 100 \% \qquad ( 79 )$$

where T is the temperature (in Celsius) of the volume of the air/water-vapor mixture, T_{d} is the dew point (in Celsius) of the volume, c=17.271 (no units), and b=237.700° C. For example, where T=T_{d}, then RH=1=100%, which means that the air is saturated, and can accommodate no more water vapor. And as an example of how relative humidity RH is perceived by humans, an RH higher than about 50% with an air temperature of about 32° C. (˜90° F.) or higher is considered oppressive (e.g., “muggy,” or “sticky”) by most humans.

In step 272 of the flow diagram 270 of FIG. 27, a computing apparatus determines the signals [symbol] for the air above the surface regions 202 (FIG. 23). For example, the computing apparatus computes each element L₁-L_{α} (FIG. 16) of a signal [symbol] corresponding to a surface region 202 by solving equation (79) for the corresponding air-temperature (T) and dew-point (T_{d}) elements of the signals [symbol] and [symbol] (e.g., FIG. 22), and [symbol] and [symbol] (e.g., FIG. 26), respectively, for the same surface region.

Referring to FIG. 27, alternate embodiments of the relative-humidity interpolation algorithm are contemplated. For example, some of the described steps may be omitted, other steps may be added, and the order in which the steps are performed may be altered.

FIG. 28 is a block diagram of a computing apparatus 290 according to an embodiment; the computing apparatus can perform the steps of the algorithms described above in conjunction with FIGS. 10-13, 15, 17, 19, 22, 26, and 27. Furthermore, as described above, instead of generating certain items, such as plots, curves, and points, in a physical/displayable sense, the computing apparatus 290 may generate computer representations (e.g., data arrays) of such items.

The computing apparatus 290 includes computer circuitry 292, a memory 294, one or more input devices 296, one or more output devices 298, and one or more data-storage devices 300. The computing apparatus 290 may be disposed on a single integrated-circuit (IC) die (e.g., a system on a chip (SOC)), or may be disposed on multiple dies or devices.

The computer circuitry 292 can perform various computing functions, such as executing specific software to perform specific calculations or tasks. For example, the computer circuitry 292 can execute software that causes the computing apparatus 290 to perform one or more of the steps of the algorithms described above in conjunction with FIGS. 10-13, 15, 17, 19, 22, 26, and 27, and can include hardware that performs, or causes the computing apparatus 290 to perform, one or more of these steps. The computer circuitry 292 can include conventional software-executing processing circuitry such as one or more microcontrollers and one or more microprocessors, and can include conventional hardwired processing circuit such as one or more application-specific integrated circuits (ASICs) and one or more field-programmable gate arrays (FPGAs). Furthermore, the computer circuitry may include one or more hardware modules, or may execute one or more software module, where each module performs a respective one or more functions.

The memory 294 can store data, and is coupled to the computer circuitry 292 via one or more of address buses, data buses, control buses, or other buses. The computer circuitry 292 can read data from, and write data to, the memory 294, which can be any type of volatile or non-volatile memory, and which may include one or more memory components. For example, the memory 294 may store software instructions that the computer circuitry 292 can fetch and execute. Furthermore, the memory 294 may serve as working memory while the computer circuitry 292 is performing calculations, and may also store data before or after interpolation of the data.

The one or more input devices 296 are coupled to the computer circuitry 292 and can include, e.g., a keyboard, a mouse, or a network connector for coupling the computing apparatus 290 to a network or the internet, and may receive data such as a signal (e.g., a land-temperature signal [symbol] ) having empty portions to be interpolated according to, e.g., one or more of the algorithms described above in conjunction with FIGS. 10-13, 15, 17, 19, 22, 26, and 27.

The one or more output devices 298 are coupled to the computer circuitry 292 and can include, e.g., a printer, one or more displays, or a network connector for coupling the computing apparatus 290 to a network or the internet, and may provide data such as a signal (e.g., a land-temperature signal [symbol] ) having portions that the computing apparatus 290 interpolated according to, e.g., one or more of the algorithms described above in conjunction with FIGS. 10-13, 15, 17, 19, 22, 26, and 27.

And the one or more storage devices 300 are coupled to the computer circuitry 292 and can include, e.g., include magnetic hard and floppy disks, tape cassettes, compact disk read-only (CD-ROMs) and compact disk read-write (CD-RW) memories, or digital video disks (DVDs), for storing data such as a signal (e.g., a land-temperature signal [symbol] ) having empty portions to be interpolated by the computing apparatus 290, or having portions that the computing apparatus 290 interpolated, according to, e.g., one or more of the algorithms described above in conjunction with FIGS. 10-13, 15, 17, 19, 22, 26, and 27.

Alternate embodiments of the computing apparatus 290 are contemplated. For example, two or more of the memory 294, one or more input devices 296, one or more output devices 298, and one or more data-storage devices 300 may be directly coupled to one another.

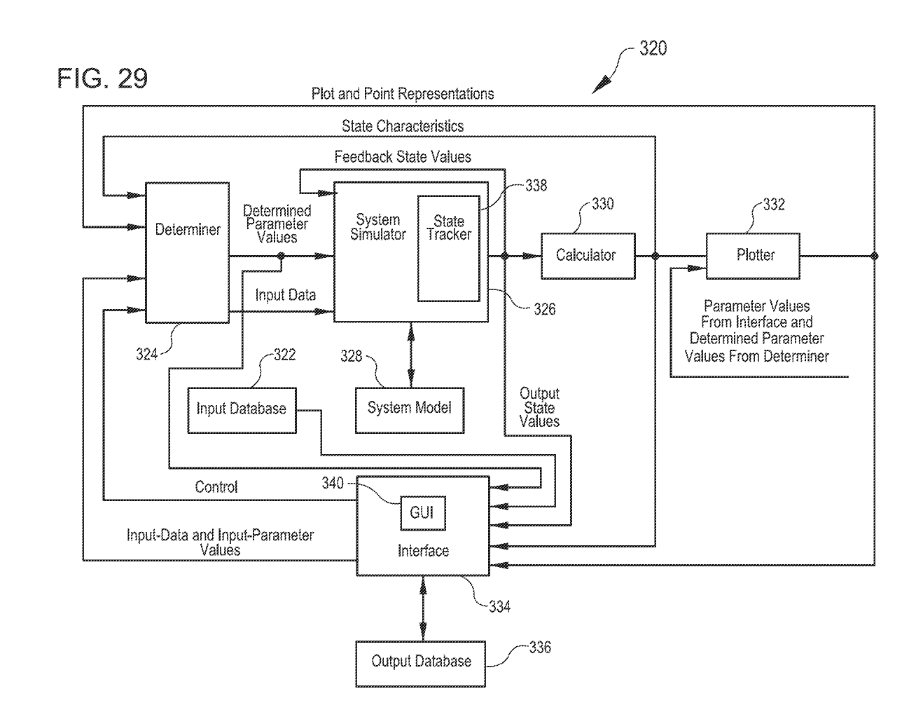

FIG. 29 is a block diagram of an embodiment of a simulation tool 320, which can simulate a system, such as a disease-transmission system, in response to data interpolated according to one of the above-described interpolation techniques, according to an embodiment. For example, the simulation tool 320 may simulate a disease-transmission system for the purpose of predicting a success rate of a disease-eradication campaign, and may use, as system inputs, land temperatures, air temperatures, dew points, and relative-humidities that have been interpolated and corrected, and in some cases calculated, as described above. The simulation tool 320 is described below, and is further described in U.S. patent application Ser. Nos. 13/199,040, 13/199,044, and 13/199,039, which were previously incorporated by reference.

The simulation tool 320 includes an input database 322, a next-parameter-value determiner 324, a system simulator 326, a system model 328, a state-characteristic calculator 330, a platter 332, an interface 334, and an output database 336. The simulator tool 320 may be installed on, or may include, a computing apparatus (e.g., the computing apparatus 290 of FIG. 28) that is configured by software, firmware, hardware, or a combination or sub-combination of software, firmware, and hardware, to perform the functions of the simulation tool, including the functions of the input database 322, determiner 324, simulator 326, system model 328, calculator 330, plotter 332, interface 334, and output database 336. These and other components of the simulation tool 320 may be separately identifiable software or firmware modules or circuits, or they may be logical components of modules or circuits that perform the functions of more than one of these components. For example, the simulation tool 320 may include means for performing the functions of these and other tool components, and these means may be may be separately identifiable software or firmware modules or circuits, or they may be modules or circuits that perform the functions of more than one of these tool components.

The input database 322 stores input data regarding the system to be simulated, the conditions of the environment in which the system is disposed, and other quantities that may influence the system. For example, the input database 322 may store, e.g., topographical information, weather information, and sunrise/sunset information, for a geographical region; such information may include data interpolated according to one of the above-described techniques. In an embodiment, the input database 322 may store data that an operator of the tool 320 does not wish to manipulate as an input parameter, e.g., because such data (e.g., topographical information) is fixed and not manipulatable in the real world.

The next-parameter-value determiner 324 determines a next value of one or more input parameters in a manner that, compared to a conventional simulation tool, allows the simulation tool 320 to hone in more quickly on a set of parameter values that corresponds to a sought-after result, or to determine more quickly that no such set of parameter values exists; for example, the determiner may allow the simulation tool to hone in on a set of parameter values, or to determine that no such set of values exists, with fewer simulation runs than a conventional simulation tool. The ID determiner 324 may determine such next-parameter values in response to one or more state-characteristic values from the calculator 330, and from the representations of one or more result-surface plots and one or more level sets (described below) from the plotter 332. The operation of the determiner 324 is described in more detail in conjunction with FIGS. 10-27 of U.S. patent application Ser. Nos. 13/199,040, 13/199,044, and 13/199,039, which were previously incorporated by reference.

The system simulator 326 includes a state tracker 338, and, referring to FIG. 3 of U.S. patent application Ser. Nos. 13/199,040, 13/199,044, and 13/199,039, which were previously incorporated by reference, propagates the x states S₀-Sₓ₋₁ of the simulated system through time in z discrete steps t₀-t_{z-1} in response to one or more input-data values and one or more input-parameter values from the determiner 324, and one or more output-state values fed back to the input of the simulator. The simulator 326 propagates the states S through time by determining the respective value that each state S has at each time step t in response to the system model 328. For example, for the simulation of a malaria-transmission system, the model 328 may define influences that environmental conditions or other quantities have on the number of infected individuals (a state of the simulated system) at a time t; examples of such conditions and quantities include the number of infected individuals at the previous time step t−1, the number of infected mosquitos at t−1, the temperature and rainfall at t−1, the bed-net coverage at t−1, the percentage of individuals vaccinated at t−1, and the migration of individuals during the interval between t−1 and t. And from these defined influences and the values of these system-influencing conditions and quantities, the simulator 326 may calculate the number of infected individuals at time t; for example, the higher the temperature and rainfall at t−1, the more infected mosquitos, and thus the more infected individuals, at t. The simulator 326 may calculate the values of the other states of the simulated system in a similar manner. Furthermore, the intervals between the steps 1 may be uniform, or they may vary if event-based timing is used. Moreover, the simulator 326 may propagate some of the states S through time in uniform time steps, and may, at least effectively, propagate other states S through time in event-based non-uniform time steps. The operation of the system simulator 326 is discussed in more detail in conjunction with FIGS. 23-27 of U.S. patent application Ser. Nos. 13/199,040, 13/199,044, and 13/199,039, which were previously incorporated by reference.

The state tracker 338 keeps track of the current value of each state S of the simulated system. For example, if a state S is the number of individuals infected, then the state tracker 338 stores this number, which the simulator 326 updates at each time step. The state tracker is further described in conjunction with FIGS. 7 and 8 of U.S. patent application Ser. Nos. 13/199,040, 13/199,044, and 13/199,039, which were previously incorporated by reference.

The system model 328 may include one or more microsimulations, and may include software, firmware, hardware, or a combination or sub-combination of software, firmware, and hardware, that defines a system to be simulated by the simulator 326. For example, for a malaria-transmission system, the system model 328 may stochastically define the system states, and may define system phenomena that affect the system states such as the migration patterns of individuals, the life cycle of a malaria parasite within a human host, the life cycle of a malaria parasite within a mosquito host, the life cycle of a mosquito, the interaction between individuals and mosquitos, and the infection of individuals by mosquitos and of mosquitos by individuals. The system model 328 may also stochastically define dependencies of such system phenomena on environmental conditions and other system-influencing quantities such as temperature, rainfall, humidity, the number of infected individuals at a time step, and the number of infected mosquitos at a time step.

The calculator 330 calculates one or more characteristics of, or one or more characteristics that are otherwise related to, one or more states S of the simulated system. For example, after a number of runs of the simulator 326 at a particular set of input-parameter and input-data values for a simulated disease-elimination campaign, the calculator 330 may calculate the probability that the simulated campaign eradicated the disease, i.e., the probability that at the end of the campaign period, the number of infected individuals (and for malaria, the number of infected mosquitos) is zero. The calculator 330 may also calculate the statistical variance of or the statistical uncertainty in, this probability. The calculator 330 may make its calculation using any suitable function such as a Bayesian prior, a beta function or incomplete beta function, which are subsets of a Bayesian prior, or a binomial distribution, which is a subset of an incomplete beta function. The operation of the calculator 330 using a binomial distribution is further described in conjunction with FIG. 9 of U.S. patent application Ser. Nos. 13/199,040, 13/199,044, and 13/199,039, which were previously incorporated by reference.

The plotter 332 generates one or more conceptual plots based on one or more of the following: the values of one or more state characteristics from the calculator 330, one or more input-parameter values from the determiner 324 and the interface 334, and one or more state values output from the simulator 326—“representation” indicates that the plotter 332 need not generate an actual visual plot (although it may), but that it may instead generate a representation of a plot in, e.g., computer memory, which the simulator tool 320 includes or to which the simulator tool otherwise has access. Alternatively, the plotter 332 may receive the input-parameter values from one, not both, of the determiner 324 and interface 334. For example, the plotter 332 may generate a representation of an N-dimensional result surface having respective input parameters as N−1 of these dimensions, and having a state characteristic as the remaining dimension. As a more detailed example, assume that for a malaria-transmission system, an operator of the tool 320 is investigating the relationship among vaccination coverage (an input parameter), bed-net coverage (another input parameter), and the probability of eradicating malaria (a state characteristic). The plotter 332 may generate a representation of an N=3-dimensional result surface having vaccination coverage as a first dimension, bed-net coverage as a second dimension, and probability of elimination as a third dimension. The plotter 332 may also generate a representation of one or more level sets for this result surface, and the determiner 324 may use such a result surface and level set to determine a respective next value of the vaccination-coverage parameter, the bed-net use parameter, or of both of these parameters. Furthermore, the plotter 332 may generate other types of plots, such as a topographical plot of a region showing a time progression of the distribution of infected individuals within the region. Operation of the plotter 332, including the generation of result surfaces and level sets, is further described in conjunction with FIGS. 10-22 of U.S. patent application Ser. Nos. 13/199,040, 13/199,044, and 13/199,039, which were previously incorporated by reference.

The interface 334 interfaces other components of the simulation tool 320 to each other, and includes a graphical user interface (GUI) 340, which allows an operator to configure and to use the simulation tool.

The interface 334 may also store in the output database 336 a “snapshot” of the values of the states S of the simulated system at each time step t of the simulation period. These snapshots may allow an operator to see the development of one or more states of the simulated system as the simulator 326 propagates these states through time, and may facilitate an operator's step-by-step analysis of a simulation run or an operator's step-by-step comparison of multiple simulation runs.

Furthermore, the interface 334 may store in the output database 336 for each simulation run the state characteristics generated by the calculator 330 and the representations of the spatial points and result surfaces generated by the plotter 332.

Moreover, the interface 334 may store in the output database 336 for each simulation run the set of input-data and input-parameter values provided to the simulator 326 for the simulation run. Because this set of input values, or a subset of this set of input values, may be the coordinates of a representation of a spatial point that is generated by the plotter 332, this set of input values is sometimes referred to as the “simulation point” of the simulation run. And an operator of the simulation tool 320 may be described as using the tool to find a simulation point that corresponds to a sought-after result. Furthermore, the interface 334 may provide this set of input values to the determiner 324 for a next simulation run that may use the same set, or a slightly modified set, of input values. Providing the set of input values from a prior simulation run to the determiner 324 may eliminate the need to re-enter, manually or otherwise, all of the input-data values from the input database 324 and all of the unmodified input-parameter values via the GUI 340.

In addition, the interface 334 and GUI 340 may allow a user to configure, control, or otherwise interact with the simulation tool 320.

Furthermore, the interface 334 may allow an operator to select input-data and input-parameter values to be input to the simulator 326. For example, if an operator would like to simulate a malaria-transmission system in Madagascar, then he/she may use the interface 334 to configure the simulation tool 322 such that Madagascar-relevant input data (e.g., weather-related data, population data) from the input database 322 is provided to the simulator 326.

Moreover, an operator may select via the interface 334 input parameters to be used in a simulation, and may also select values of these input parameters. For example, for simulating a malaria-transmission system, an operator may select vaccination coverage and bed-net coverage as input parameters, and may select initial values for these coverages.

In addition, an operator may configure the system model 328 via the interface 334. For example, there is more than one malaria parasite, and one parasite may behave differently under given conditions than another parasite. Therefore, an operator may configure the model 328 for a particular malaria parasite. Or, if individuals of one type (e.g., child vs. adult) react, on average, differently to a malaria parasite than individuals of another type, then an operator may provide the model 328 with the percentages of each type of individual in a region of interest (alternatively, these percentages may come from the input database 322).

Furthermore, an operator may configure the determiner 324 via the interface 334. For example, an operator may specify the parameter or parameters for which the determiner 324 will determine a next value after a set of simulation runs, and may also specify an algorithm or other criteria that the determiner uses to determine such next value(s). An example of such an algorithm is described in conjunction with FIGS. 10-22 of U.S. patent application Ser. Nos. 13/199,040, 13/199,044, and 13/199,039, which were previously incorporated by reference.

Moreover, an operator may, via the interface 334, specify one or more state characteristics and configure the calculator 330 to generate the values of the specified one or more characteristics.

In addition, an operator may, via the interface 334, specify one or more plots and configure the plotter 332 to generate representations of the specified one or more plots.

Furthermore, an operator may configure, via the interface 334, the GUI 340 to display tool-generated quantities such as one or more state values from the simulator 326, one or more state-characteristic values from the calculator 330, or one or more plots from the plotter 332.

The output database 336 may store the state-value snapshots from the simulator 326, the state-characteristic values from the calculator 330, the representations of the points and plots from the plotter 332, and the sets of input-data and input-parameter values input to the simulator as described above, and may also store other data.

Still referring to FIG. 29, alternate embodiments of the simulation tool 320 are contemplated. For example, the system model 328 may be part of the system simulator 326.

From the foregoing it will be appreciated that, although specific embodiments have been described herein for purposes of illustration, various modifications may be made without deviating from the spirit and scope of the disclosure. Furthermore, where an alternative is disclosed for a particular embodiment, this alternative may also apply to other embodiments even if not specifically stated.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. An apparatus, comprising:
a first component determiner configured to determine a component of a first signal; and
an interpolator configured to interpolate a portion of a second signal in response to the component of the first signal.

2. The apparatus of claim 1 wherein the component of the first signal includes an altitude-component of the first signal.

3. The apparatus of claim 1 wherein the component of the first signal includes an approximately zero-altitude component of the first signal.

4. The apparatus of claim 1 wherein the first and second signals represent a physical quantity.

5. The apparatus of claim 1 wherein the first and second signals represent dew point.

6. The apparatus of claim 1 wherein the first and second signals respectively represent dew points of first and second regions above a surface of a celestial body.

7. The apparatus of claim 1 wherein the first and second signals respectively represent dew points of first and second regions of an atmosphere.

8. The apparatus of claim 1 wherein:
the first and second signals respectively include first and second digital signals; and
the interpolated portion of the second signal includes an interpolated sample of the second digital signal.

9. The apparatus of claim 1 wherein:
the first and second signals respectively include first and second sets of values; and
the interpolated portion of the second signal includes an interpolated value of the second set.

10. (canceled)

11. The apparatus of claim 1 wherein the portion of the second signal includes an empty portion of the second signal.

12-14. (canceled)

15. The apparatus of claim 1, further comprising:
a second component determiner configured to determine a second component of the first signal; and
wherein the first component determiner is configured to determine the first component of the first signal in response to the first signal and to the second component of the first signal.

16. The apparatus of claim 1, further comprising:
a lapse-rate-component determiner configured to determine a lapse-rate component of the first signal; and
wherein the first component determiner is configured to determine an altitude component of the first signal as the first component of the first signal in response to a difference between the first signal and the lapse-rate component of the first signal.

17. (canceled)

18. The apparatus of claim 1, further comprising:
a relationship determiner configured to determine a relationship between the first and second signals; and
wherein the interpolator is configured to interpolate the portion of the second signal in response to the relationship.

19-21. (canceled)

22. The apparatus of claim 1, further comprising:
a correlator configured to determine a representation of a semi-variogram of a correlation between the first and second signals; and
wherein the interpolator is configured to interpolate the portion of the second signal in response to the representation of the semi-variogram.

23. The apparatus of claim 1 wherein the interpolator is configured:
to interpolate a component of the second signal; and
to interpolate the portion of the second signal in response to the interpolated component of the second signal and a value associated with the portion of the second signal.

24. The apparatus of claim 1, further comprising:
a lapse-rate-component determiner configured to determine a lapse-rate component of the second signal;
wherein the first component determiner is configured to determine an altitude component of the first signal; and
wherein the interpolator is configured:
to interpolate an altitude component of the second signal in response to the altitude component of the first signal; and
to interpolate the portion of the second signal in response to the lapse-rate component of the second signal and the interpolated altitude component of the second signal.

25. The apparatus of claim 1, further comprising:
wherein the second signal corresponds to a location;
a lapse-rate-component determiner configured to determine a lapse-rate component associated with the location;
wherein the first component determiner is configured to determine an altitude component of the first signal; and
wherein the interpolator is configured:
to interpolate an altitude component associated with the location in response to the altitude component of the first signal; and
to interpolate the portion of the second signal in response to the lapse-rate component associated with the location and the interpolated altitude component associated with the location.

26. The apparatus of claim 1 wherein the first and second signals are a same signal.

27. (canceled)

28. The apparatus of claim 1, further comprising:
wherein the second signal represents dew point; and
a relative-humidity determiner configured to determine a relative-humidity signal from the second signal including the interpolated portion.

29. The apparatus of claim 1, further comprising:
wherein the component of the first signal is a function of a variable, extends over a range of the variable, and has an empty portion and a non-empty portion;
a transformer configured to convert the component of the first signal over the range of the variable into a transformed signal that is a function of another variable;
an inverse transformer configured to convert the transformed signal into a periodic component of the component that is a function of the variable and that has an non-empty portion corresponding to the empty portion of the first signal; and
wherein the interpolator is configured:
to determine a difference between the component of the first signal and the periodic component of the component, the difference corresponding to the non-empty portion of the component;
to interpolate a difference value corresponding to the empty portion of the component in response to the difference; and
to interpolate the empty portion of the component of the first signal in response to the periodic component of the component and the difference value.

30. The apparatus of claim 1, further comprising:
wherein the component of the first signal is a function of time, extends over a range of time, and has an empty portion at an empty-portion time and a non-empty portion at a non-empty-portion time;
a transformer configured to convert the component of the first signal over the range of time into a transformed signal that is a function of frequency;
an inverse transformer configured to convert the transformed signal into a periodic component of the component that is a function of time and that has a non-empty portion at the empty-portion time; and
wherein the interpolator is configured:
to determine a difference between the component of the first signal and the periodic component of the component, the difference corresponding to the non-empty-portion time of the component;
to interpolate a difference value corresponding to the empty-portion time in response to the difference; and
to interpolate the empty portion of the component of the first signal in response to the periodic component of the component and the difference value.

31. The apparatus of claim 30 wherein the transformed signal has a fundamental frequency of approximately one year.

32. The apparatus of claim 30 wherein the periodic component of the component has a fundamental frequency of approximately one year.

33. An apparatus, comprising:
an altitude-component determiner configured to determine an altitude component of a first signal;
a lapse-rate-component determiner configured to determine a lapse-rate component of a second signal having an empty portion; and
an interpolator configured
to interpolate an altitude component of the second signal in response to the altitude component of the first signal, and
to interpolate the empty portion of the second signal in response to the lapse-rate and altitude components of the second signal.

34-57. (canceled)

58. The apparatus of claim 33, further comprising:
wherein the second signal represents dew point; and
a relative-humidity determiner configured to determine a relative-humidity signal from the second signal including the interpolated portion.

59. The apparatus of claim 33, further comprising:
wherein the altitude component of the first signal is a function of a variable, extends over a range of the variable, and has an empty portion and a non-empty portion,
a transformer configured to convert the altitude component of the first signal over the range of the variable into a transformed signal that is a function of another variable,
an inverse transformer configured to convert the transformed signal into a periodic component of the altitude component, the periodic component being a function of the variable and having an non-empty portion corresponding to the empty portion of the altitude component; and
wherein the interpolator is configured
to determine a difference between the altitude component of the first signal and the periodic component of the altitude component, the difference corresponding to the non-empty portion of the altitude component;
to interpolate a difference value corresponding to the empty portion of the altitude component in response to the difference; and
to interpolate the empty portion of the altitude component of the first signal in response to the periodic component of the altitude component and the difference value.

60. The apparatus of claim 33, further comprising:
wherein the altitude component of the first signal is a function of time, extends over a range of time, and has an empty portion at an empty-portion time and a non-empty portion at a non-empty-portion time;
a transformer configured to convert the altitude component of the first signal over the range of time into a transformed signal that is a function of frequency;
an inverse transformer configured to convert the transformed signal into a periodic component of the altitude component, the periodic component being a function of time and having a non-empty portion at the empty-portion time; and
wherein the interpolator is configured
to determine a difference between the altitude component of the first signal and the periodic component of the altitude component, the difference corresponding to the non-empty-portion time of the altitude component;
to interpolate a difference value corresponding to the empty-portion time in response to the difference; and
to interpolate the empty portion of the altitude component of the first signal in response to the periodic component of the altitude component and the difference value.

61-62. (canceled)

63. An apparatus, comprising:
means for determining a component of a first signal; and
means for interpolating a portion of a second signal in response to the component of the first signal.

64-124. (canceled)

125. A method, comprising:
determining a component of a first signal; and
interpolating a portion of a second signal in response to the component of the first signal.

126-186. (canceled)

187. A tangible computer-readable medium storing instructions that, when executed by at least one computing apparatus, cause the at least one computing apparatus:
to determine a component of a first signal; and
to interpolate a portion of a second signal in response to the component of the first signal.

188-248. (canceled)
